(19) United States
(12) Patent Application Publication
(10) Pub. No.: US 2024/0376511 A1
(43) Pub. Date: Nov. 14, 2024

(54) COMPOSITIONS AND METHODS FOR ENHANCED PROTEIN PRODUCTION IN FUNGAL CELLS

(71) Applicant: DANISCO US INC., Palo Alto, CA (US)

(72) Inventors: Barbara Urszula KOZAK, Utrecht (NL); Igor NIKOLAEV, Noordwijk (NL); Jonathan M. PALMER, Palo Alto, CA (US); Nuria BARRAJÓN SIMANCAS, Leiden (NL)

(21) Appl. No.: 18/578,223

(22) PCT Filed: Jun. 30, 2022
(86) PCT No.: PCT/US2022/035655

§ 371 Date: Jan. 10, 2024

Related U.S. Application Data
(60) Provisional application No. 63/223,277, filed on Jul. 19, 2021.

(51) Int. Cl. C12P 21/02; C12N 1/14; C12N 9/16; C12N 9/30; C12N 15/80; C12R 1/885

(52) U.S. Cl. CPC C12P 21/02; C12N 1/14; C12N 9/16; C12N 9/242; C12N 15/80; C12R 2001/885; C12Y 301/03008; C12Y 302/01001

(57) ABSTRACT

The present disclosure is generally related to recombinant fungal cells (strains) for use in the commercial scale production of proteins (polypeptides) of interest. Certain embodiments are therefore related to recombinant fungal cells (strains) producing proteins of interest and overexpressing one or more gene(s) encoding a regulatory protein of the disclosure.

### CROSS REFERENCE TO RELATED APPLICATIONS

This application claims benefit to U.S. Provisional Patent Application No. 63/223,277, filed Jul. 19, 2021, which is incorporated herein by reference in its entirety.

### FIELD

The present disclosure is generally related to the fields of molecular biology, biochemistry, regulatory proteins, industrial fermentation, protein production, filamentous fungi and the like. Certain embodiments of the disclosure are related to modified (mutant) filamentous fungal cells and methods thereof for use in the enhanced production of proteins of interest.

### REFERENCE TO A SEQUENCE LISTING

The sequence listing text file submitted herewith contains the file “NB41848-WO-PCT_SequenceListing.txt” created on Jun. 27, 2022, which is 404 kilobytes (KB) in size. This sequence listing complies with 37 C.F.R. § 1.52(e) and is incorporated herein by reference in its entirety.

### BACKGROUND

Many of the biopolymer degrading hydrolytic enzymes, such as cellulases, hemi-cellulases, ligninases, pectinases and the like have received attention because of their potential applications in food, feed, textile, pulp and paper industries and the like. For example, industrial filamentous fungal production strains, in particular Aspergillus and Trichoderma strains, can produce high amounts of these extracellular enzymes. Likewise, the existence of hypersecreting strains and strong promoters, such as cellulase (gene) promoters, render filamentous fungal cells particularly suitable for heterologous protein production.

Thus, filamentous fungi are capable of expressing native and heterologous proteins to high levels, making them well-suited for the large-scale production of enzymes and other proteins for industrial, pharmaceutical, animal health, and food and beverage applications and the like. Despite current knowledge in the art related to filamentous fungal strains, there is a continued and ongoing need in the art for improved strains for use in the production of proteins of interest. As described hereinafter, the recombinant filamentous fungal cells (strains) of the disclosure are well-suited for use in industrial scale fermentation processes for the enhanced expression/production of endogenous and/or heterologous proteins of interest.

### SUMMARY

As generally set forth herein, certain embodiments of the disclosure are related to recombinant fungal cells capable of producing increased amounts of proteins of interest. More particularly, as described and exemplified hereafter, Applicant has designed/constructed exemplary T. reesei reporter strains and modified (recombinant) strains thereof overexpressing one or more regulatory proteins of the disclosure. In the instant examples, the reporter strains were constructed to express/produce two (2) proteins of interest (i.e., reporter proteins; e.g., a phytase and an amylase).

Thus, certain embodiments of the disclosure provide, inter alia, recombinant fungal cells overexpressing one or more proteins comprising at least 80% identity to a Trichoderma reesei protein shown in TABLE 1, and/or overexpressing one or more proteins comprising at least 80% identity to a protein ortholog shown in TABLE 6. Thus, in certain embodiments, recombinant fungal cells of the disclosure overexpress a combination of at least two proteins, at least three proteins, etc., comprising at least 80% identity to Trichoderma reesei gene sequences shown in TABLE 1, and/or at least 80% identity to an orthologous gene sequence shown in TABLE 6.

Certain other aspects of the disclosure relate to recombinant polynucleotides encoding such regulatory proteins, whereas other aspects relate to recombinant polynucleotides encoding proteins of interest. In other related embodiments, the disclosure provides recombinant fungal cell comprising introduced polynucleotides encoding one or more regulatory proteins and/or one or more proteins of interest. For example, in certain embodiments, recombinant fungal cells of the disclosure produce protein of interest such as enzymes, antibodies, receptor proteins, animal feed proteins, human food proteins and the like.

In certain other embodiments, Applicant describes methods for producing increased amounts of proteins of interest in one or more recombinant cells of the disclosure. For example, certain aspects are related to methods for constructing recombinant fungal cells expressing/producing one or more proteins of interest. In certain embodiments of the methods, recombinant fungal cells expressing/producing a protein of interest (POI) comprise one or more introduced polynucleotides (e.g., expression cassettes) encoding one or more regulatory proteins of the disclosure.

### BRIEF DESCRIPTION OF THE DRAWINGS

FIG. 1 shows a vector map of pBKT030.

FIG. 2 shows the amount of amylase reporter protein produced during small scale (2 L) fermentations of the modified strains overexpressing a single regulatory protein (FIG. 2; SEQ ID NO: 30, 65, 68 or 101). More particularly, the amount of reporter protein produced by the parental strain (FIG. 2; control) throughout the fermentation was set at 1, and the relative amounts of protein produced by each strain at each time point was plotted.

FIG. 3 shows the amount of reporter protein produced during small scale (2 L) fermentations, by the modified strains overexpressing two regulatory factors. More particularly, the amount of protein produced by the parental strain (FIG. 3; control) throughout the fermentation was set at 1, and the relative amounts of protein produced by each strain at each time point was plotted.

FIG. 4 shows the sporulation level of the modified strain overexpressing regulatory protein SEQ ID NO: 68 (bottom plate) and the control strain not overexpressing any regulatory proteins (top plate).

### BRIEF DESCRIPTION OF THE BIOLOGICAL SEQUENCES

SEQ ID NO: 1 is a T. reesei DNA sequence encoding the regulatory protein of SEQ ID NO: 2.

SEQ ID NO: 2 is the predicted amino acid sequence of the regulatory protein encoded by SEQ ID NO: 1.

SEQ ID NO: 3 is the predicted amino acid sequence of the DNA biding domain (DBD) present in the regulatory protein of SEQ ID NO: 2.

SEQ ID NO: 4 is a T. reesei DNA sequence encoding the regulatory protein of SEQ ID NO: 5.

SEQ ID NO: 5 is the predicted amino acid sequence of the regulatory protein encoded by SEQ ID NO: 4.

SEQ ID NO: 6 is the predicted amino acid sequence of the DBD present in the regulatory protein of SEQ ID NO: 5.

SEQ ID NO: 7 is a T. reesei DNA sequence encoding the regulatory protein of SEQ ID NO: 8.

SEQ ID NO: 8 is the predicted amino acid sequence of the regulatory protein encoded by SEQ ID NO: 7.

SEQ ID NO: 9 is the predicted amino acid sequence of the DBD present in the regulatory protein of SEQ ID NO: 8.

SEQ ID NO: 10 is a T. reesei DNA sequence encoding the regulatory protein of SEQ ID NO: 11.

SEQ ID NO: 11 is the predicted amino acid sequence of the regulatory protein encoded by SEQ ID NO: 10.

SEQ ID NO: 12 is a T. reesei DNA sequence encoding the regulatory protein of SEQ ID NO: 13.

SEQ ID NO: 13 is the predicted amino acid sequence of the regulatory protein encoded by SEQ ID NO: 12.

SEQ ID NO: 14 is the predicted amino acid sequence of the DBD present in the regulatory protein of SEQ ID NO: 13.

SEQ ID NO: 15 is a T. reesei DNA sequence encoding the regulatory protein of SEQ ID NO: 16.

SEQ ID NO: 16 is the predicted amino acid sequence of the regulatory protein encoded by SEQ ID NO: 15.

SEQ ID NO: 17 is the predicted amino acid sequence of the DBD present in the regulatory protein of SEQ ID NO: 16.

SEQ ID NO: 18 is a T. reesei DNA sequence encoding the regulatory protein of SEQ ID NO: 19.

SEQ ID NO: 19 is the predicted amino acid sequence of the regulatory protein encoded by SEQ ID NO: 18.

SEQ ID NO: 20 is the predicted amino acid sequence of the DBD present in the regulatory protein of SEQ ID NO: 19.

SEQ ID NO: 21 is a T. reesei DNA sequence encoding the regulatory protein of SEQ ID NO: 22.

SEQ ID NO: 22 is the predicted amino acid sequence of the regulatory protein encoded by SEQ ID NO: 21.

SEQ ID NO: 23 is a T. reesei DNA sequence encoding the regulatory protein of SEQ ID NO: 24.

SEQ ID NO: 24 is the predicted amino acid sequence of the regulatory protein encoded by SEQ ID NO: 23.

SEQ ID NO: 25 is a T. reesei DNA sequence encoding the regulatory protein of SEQ ID NO: 26.

SEQ ID NO: 26 is the predicted amino acid sequence of the regulatory protein encoded by SEQ ID NO: 25

SEQ ID NO: 27 is the predicted amino acid sequence of the DBD present in the regulatory protein of SEQ ID NO: 26.

SEQ ID NO: 28 is a T. reesei DNA sequence encoding the regulatory protein of SEQ ID NO: 29.

SEQ ID NO: 29 is the predicted amino acid sequence of the regulatory protein encoded by SEQ ID NO: 28.

SEQ ID NO: 30 is a T. reesei DNA sequence encoding the regulatory protein of SEQ ID NO: 31.

SEQ ID NO: 31 is the predicted amino acid sequence of the regulatory protein encoded by SEQ ID NO: 30.

SEQ ID NO: 32 is the predicted amino acid sequence of the DBD present in the regulatory protein of SEQ ID NO: 31.

SEQ ID NO: 33 is a T. reesei DNA sequence encoding the regulatory protein of SEQ ID NO: 34.

SEQ ID NO: 34 is the predicted amino acid sequence of the regulatory protein encoded by SEQ ID NO: 33.

SEQ ID NO: 35 is the predicted amino acid sequence of the DBD present in the regulatory protein of SEQ ID NO: 34.

SEQ ID NO: 36 is a T. reesei DNA sequence encoding the regulatory protein of SEQ ID NO: 37.

SEQ ID NO: 37 is the predicted amino acid sequence of the regulatory protein encoded by SEQ ID NO: 36.

SEQ ID NO: 38 is the predicted amino acid sequence of the DBD present in the regulatory protein of SEQ ID NO: 37.

SEQ ID NO: 39 is a T. reesei DNA sequence encoding the regulatory protein of SEQ ID NO: 40.

SEQ ID NO: 40 is the predicted amino acid sequence of the regulatory protein encoded by SEQ ID NO: 39.

SEQ ID NO: 41 is the predicted amino acid sequence of the DBD present in the regulatory protein of SEQ ID NO: 40.

SEQ ID NO: 42 is a T. reesei DNA sequence encoding the regulatory protein of SEQ ID NO: 43.

SEQ ID NO: 43 is the predicted amino acid sequence of the regulatory protein encoded by SEQ ID NO: 42.

SEQ ID NO: 44 is the predicted amino acid sequence of the DBD present in the regulatory protein of SEQ ID NO: 43.

SEQ ID NO: 45 is a T. reesei DNA sequence encoding the regulatory protein of SEQ ID NO: 46.

SEQ ID NO: 46 is the predicted amino acid sequence of the regulatory protein encoded by SEQ ID NO: 45.

SEQ ID NO: 47 is the predicted amino acid sequence of the DBD present in the regulatory protein of SEQ ID NO: 46.

SEQ ID NO: 48 is a T. reesei DNA sequence encoding the regulatory protein of SEQ ID NO: 49.

SEQ ID NO: 49 is the predicted amino acid sequence of the regulatory protein encoded by SEQ ID NO: 48.

SEQ ID NO: 50 is the predicted amino acid sequence of the DBD present in the regulatory protein of SEQ ID NO: 49.

SEQ ID NO: 51 is a T. reesei DNA sequence encoding the regulatory protein of SEQ ID NO: 52.

SEQ ID NO: 52 is the predicted amino acid sequence of the regulatory protein encoded by SEQ ID NO: 51.

SEQ ID NO: 53 is the predicted amino acid sequence of the DBD present in the regulatory protein of SEQ ID NO: 52.

SEQ ID NO: 54 is a T. reesei DNA sequence encoding the regulatory protein of SEQ ID NO: 55.

SEQ ID NO: 55 is the predicted amino acid sequence of the regulatory protein encoded by SEQ ID NO: 54.

SEQ ID NO: 56 is a T. reesei DNA sequence encoding the regulatory protein of SEQ ID NO: 57.

SEQ ID NO: 57 is the predicted amino acid sequence of the regulatory protein encoded by SEQ ID NO: 56.

SEQ ID NO: 58 is the predicted amino acid sequence of the DBD present in the regulatory protein of SEQ ID NO: 57.

SEQ ID NO: 59 is a T. reesei DNA sequence encoding the regulatory protein of SEQ ID NO: 60.

SEQ ID NO: 60 is the predicted amino acid sequence of the regulatory protein encoded by SEQ ID NO: 59.

SEQ ID NO: 61 is the predicted amino acid sequence of the DBD present in the regulatory protein of SEQ ID NO: 60.

SEQ ID NO: 62 is a T. reesei DNA sequence encoding the regulatory protein of SEQ ID NO: 63.

SEQ ID NO: 63 is the predicted amino acid sequence of the regulatory protein encoded by SEQ ID NO: 62.

SEQ ID NO: 64 is the predicted amino acid sequence of the DBD present in the regulatory protein of SEQ ID NO: 63.

SEQ ID NO: 65 is a T. reesei DNA sequence encoding the regulatory protein of SEQ ID NO: 66.

SEQ ID NO: 66 is the predicted amino acid sequence of the regulatory protein encoded by SEQ ID NO: 65.

SEQ ID NO: 67 is the predicted amino acid sequence of the DBD present in the regulatory protein of SEQ ID NO: 66.

SEQ ID NO: 68 is a T. reesei DNA sequence encoding the regulatory protein of SEQ ID NO: 69.

SEQ ID NO: 69 is the predicted amino acid sequence of the regulatory protein encoded by SEQ ID NO: 68.

SEQ ID NO: 70 is the predicted amino acid sequence of the DBD present in the regulatory protein of SEQ ID NO: 69.

SEQ ID NO: 71 is a T. reesei DNA sequence encoding the regulatory protein of SEQ ID NO: 72.

SEQ ID NO: 72 is the predicted amino acid sequence of the regulatory protein encoded by SEQ ID NO: 71.

SEQ ID NO: 73 is the predicted amino acid sequence of the DBD present in the regulatory protein of SEQ ID NO: 72.

SEQ ID NO: 74 is a T. reesei DNA sequence encoding the regulatory protein of SEQ ID NO: 75.

SEQ ID NO: 75 is the predicted amino acid sequence of the regulatory protein encoded by SEQ ID NO: 74.

SEQ ID NO: 76 is the predicted amino acid sequence of the DBD present in the regulatory protein of SEQ ID NO: 75.

SEQ ID NO: 77 is a T. reesei DNA sequence encoding the regulatory protein of SEQ ID NO: 78.

SEQ ID NO: 78 is the predicted amino acid sequence of the regulatory protein encoded by SEQ ID NO: 79.

SEQ ID NO: 79 is a T. reesei DNA sequence encoding the regulatory protein of SEQ ID NO: 80.

SEQ ID NO: 80 is the predicted amino acid sequence of the regulatory protein encoded by SEQ ID NO: 79.

SEQ ID NO: 81 is the predicted amino acid sequence of the DBD present in the regulatory protein of SEQ ID NO: 80.

SEQ ID NO: 82 is a T. reesei DNA sequence encoding the regulatory protein of SEQ ID NO: 83.

SEQ ID NO: 83 is the predicted amino acid sequence of the regulatory protein encoded by SEQ ID NO: 82.

SEQ ID NO: 84 is a T. reesei DNA sequence encoding the regulatory protein of SEQ ID NO: 85.

SEQ ID NO: 85 is the predicted amino acid sequence of the regulatory protein encoded by SEQ ID NO: 84.

SEQ ID NO: 86 is the predicted amino acid sequence of the DBD present in the regulatory protein of SEQ ID NO: 85.

SEQ ID NO: 87 is a T. reesei DNA sequence encoding the regulatory protein of SEQ ID NO: 88.

SEQ ID NO: 88 is the predicted amino acid sequence of the regulatory protein encoded by SEQ ID NO: 87.

SEQ ID NO: 89 is the predicted amino acid sequence of the DBD present in the regulatory protein of SEQ ID NO: 88.

SEQ ID NO: 90 is a T. reesei DNA sequence encoding the regulatory protein of SEQ ID NO: 91.

SEQ ID NO: 91 is the predicted amino acid sequence of the regulatory protein encoded by SEQ ID NO: 90.

SEQ ID NO: 92 is the predicted amino acid sequence of a first DBD present in the regulatory protein of SEQ ID NO: 91.

SEQ ID NO: 93 is the predicted amino acid sequence of a second DBD present in the regulatory protein of SEQ ID NO: 91.

SEQ ID NO: 94 is a T. reesei DNA sequence encoding the regulatory protein of SEQ ID NO: 95.

SEQ ID NO: 95 is the predicted amino acid sequence of the regulatory protein encoded by SEQ ID NO: 94.

SEQ ID NO: 96 is the predicted amino acid sequence of the DBD present in the regulatory protein of SEQ ID NO: 95.

SEQ ID NO: 97 is a T. reesei DNA sequence encoding the regulatory protein of SEQ ID NO: 98.

SEQ ID NO: 98 is the predicted amino acid sequence of the regulatory protein encoded by SEQ ID NO: 97.

SEQ ID NO: 99 is a T. reesei DNA sequence encoding the regulatory protein of SEQ ID NO: 100.

SEQ ID NO: 100 is the predicted amino acid sequence of the regulatory protein encoded by SEQ ID NO: 99.

SEQ ID NO: 101 is a T. reesei DNA sequence encoding the regulatory protein of SEQ ID NO: 102.

SEQ ID NO: 102 is the predicted amino acid sequence of the regulatory protein encoded by SEQ ID NO: 101.

SEQ ID NO: 103 is the predicted amino acid sequence of a first DBD present in the regulatory protein of SEQ ID NO: 102.

SEQ ID NO: 104 is the predicted amino acid sequence of a second DBD present in the regulatory protein of SEQ ID NO: 102.

SEQ ID NO: 105 is a T. reesei DNA sequence encoding the regulatory protein of SEQ ID NO: 106.

SEQ ID NO: 106 is the predicted amino acid sequence of the regulatory protein encoded by SEQ ID NO: 105.

SEQ ID NO: 107 is the predicted amino acid sequence of the DBD present in the regulatory protein of SEQ ID NO: 106.

SEQ ID NO: 108 is a T. reesei DNA sequence encoding the regulatory protein of SEQ ID NO: 109.

SEQ ID NO: 109 is the predicted amino acid sequence of the regulatory protein encoded by SEQ ID NO: 108.

SEQ ID NO: 110 is a T. reesei DNA sequence encoding the regulatory protein of SEQ ID NO: 111.

SEQ ID NO: 111 is the predicted amino acid sequence of the regulatory protein encoded by SEQ ID NO: 110.

SEQ ID NO: 112 is the predicted amino acid sequence of the DBD present in the regulatory protein of SEQ ID NO: 101.

SEQ ID NO: 113 is a T. reesei DNA sequence encoding the regulatory protein of SEQ ID NO: 114.

SEQ ID NO: 114 is the predicted amino acid sequence of the regulatory protein encoded by SEQ ID NO: 113.

SEQ ID NO: 115 is the predicted amino acid sequence of the DBD present in the regulatory protein of SEQ ID NO: 114.

SEQ ID NO: 116 is a T. reesei DNA sequence encoding the regulatory protein of SEQ ID NO: 117.

SEQ ID NO: 117 is the predicted amino acid sequence of the regulatory protein encoded by SEQ ID NO: 116.

SEQ ID NO: 118 is the predicted amino acid sequence of the DBD present in the regulatory protein of SEQ ID NO: 117.

SEQ ID NO: 119 is a T. reesei DNA sequence encoding the regulatory protein of SEQ ID NO: 120.

SEQ ID NO: 120 is the predicted amino acid sequence of the regulatory protein encoded by SEQ ID NO: 119.

SEQ ID NO: 121 is the predicted amino acid sequence of the DBD present in the regulatory protein of SEQ ID NO: 120.

SEQ ID NO: 122 is a T. reesei DNA sequence encoding the regulatory protein of SEQ ID NO: 123.

SEQ ID NO: 123 is the predicted amino acid sequence of the regulatory protein encoded by SEQ ID NO: 122.

SEQ ID NO: 124 is the predicted amino acid sequence of the DBD present in the regulatory protein of SEQ ID NO: 123.

SEQ ID NO: 125 is a T. reesei DNA sequence encoding the regulatory protein of SEQ ID NO: 126.

SEQ ID NO: 126 is the predicted amino acid sequence of the regulatory protein encoded by SEQ ID NO: 125.

SEQ ID NO: 127 is the predicted amino acid sequence of the DBD present in the regulatory protein of SEQ ID NO: 126.

SEQ ID NO: 128 is a T. reesei DNA sequence encoding the regulatory protein of SEQ ID NO: 129.

SEQ ID NO: 129 is the predicted amino acid sequence of the regulatory protein encoded by SEQ ID NO: 128.

SEQ ID NO: 130 is the predicted amino acid sequence of the DBD present in the regulatory protein of SEQ ID NO: 129.

SEQ ID NO: 131 is a T. reesei DNA sequence encoding the regulatory protein of SEQ ID NO: 132.

SEQ ID NO: 132 is the predicted amino acid sequence of the regulatory protein encoded by SEQ ID NO: 131.

SEQ ID NO: 133 is the predicted amino acid sequence of the DBD present in the regulatory protein of SEQ ID NO: 132.

SEQ ID NO: 134 is a T. reesei DNA sequence encoding the regulatory protein of SEQ ID NO: 135.

SEQ ID NO: 135 is the predicted amino acid sequence of the regulatory protein encoded by SEQ ID NO: 134.

SEQ ID NO: 136 is the predicted amino acid sequence of the DBD present in the regulatory protein of SEQ ID NO: 135.

SEQ ID NO: 137 is a T. reesei DNA sequence encoding the regulatory protein of SEQ ID NO: 138.

SEQ ID NO: 138 is the predicted amino acid sequence of the regulatory protein encoded by SEQ ID NO: 137.

SEQ ID NO: 139 is the predicted amino acid sequence of the DBD present in the regulatory protein of SEQ ID NO: 138.

SEQ ID NO: 140 is a T. reesei DNA sequence encoding the regulatory protein of SEQ ID NO: 141.

SEQ ID NO: 141 is the predicted amino acid sequence of the regulatory protein encoded by SEQ ID NO: 140.

SEQ ID NO: 142 is the predicted amino acid sequence of the DBD present in the regulatory protein of SEQ ID NO: 141.

SEQ ID NO: 143 is the predicted amino acid sequence of the DBD present in the regulatory protein of SEQ ID NO: 141.

SEQ ID NO: 144 is a T. reesei DNA sequence encoding the regulatory protein of SEQ ID NO: 145.

SEQ ID NO: 145 is the predicted amino acid sequence of the regulatory protein encoded by SEQ ID NO: 144.

SEQ ID NO: 146 is the predicted amino acid sequence of the DBD present in the regulatory protein of SEQ ID NO: 145.

SEQ ID NO: 147 is a T. reesei DNA sequence encoding the regulatory protein of SEQ ID NO: 148.

SEQ ID NO: 148 is the predicted amino acid sequence of the regulatory protein encoded by SEQ ID NO: 147.

SEQ ID NO: 149 is a T. reesei DNA sequence encoding the regulatory protein of SEQ ID NO: 150.

SEQ ID NO: 150 is the predicted amino acid sequence of the regulatory protein encoded by SEQ ID NO: 149.

SEQ ID NO: 151 is the predicted amino acid sequence of the DBD present in the regulatory protein of SEQ ID NO: 150.

SEQ ID NO: 152 is a T. reesei DNA sequence encoding the regulatory protein of SEQ ID NO: 153.

SEQ ID NO: 153 is the predicted amino acid sequence of the regulatory protein encoded by SEQ ID NO: 152.

SEQ ID NO: 154 is the predicted amino acid sequence of the DBD present in the regulatory protein of SEQ ID NO: 153.

### DETAILED DESCRIPTION

### I. Overview

As described herein, certain embodiments are related to recombinant (genetically modified) filamentous fungal cells (strains) for use in the commercial scale production of proteins (polypeptides). For example, certain embodiments are related to recombinant filamentous fungal cells overexpressing a regulatory gene comprising at least 80% identity to a Trichoderma reesei gene of SEQ ID NO: 1, 4, 7, 10, 12, 15, 18, 21, 23, 25, 28, 30, 33, 36, 39, 42, 45, 48, 51, 54, 56, 59, 62, 65, 68, 71, 74, 77, 79, 82, 84, 87, 90, 94, 97, 99, 101, 105, 108, 110, 113, 116, 119, 122, 125, 128, 131, 134, 137, 140, 144, 147, 149, and/or 152, or overexpressing a regulatory gene comprising at least 80% identity to an orthologous gene sequence shown in TABLE 6.

Certain other embodiments are related to recombinant filamentous fungal cells overexpressing a combination of at least two regulatory genes comprising at least 80% identity to a T. reesei regulatory gene sequence combination shown in TABLE 4 and/or TABLE 5. Thus, certain other embodiments are related to recombinant filamentous fungal cells producing proteins of interest and overexpressing a regulatory gene comprising at least 80% identity to a T. reesei regulatory gene of SEQ ID NO: 1, 4, 7, 10, 12, 15, 18, 21, 23, 25, 28, 30, 33, 36, 39, 42, 45, 48, 51, 54, 56, 59, 62, 65, 68, 71, 74, 77, 79, 82, 84, 87, 90, 94, 97, 99, 101, 105, 108, 110, 113, 116, 119, 122, 125, 128, 131, 134, 137, 140, 144, 147, 149, and/or 152, or overexpressing a regulatory gene comprising at least 80% identity to an orthologous gene sequence shown in TABLE 6. Certain other aspects are related to polynucleotides (e.g., expression cassettes) comprising an upstream (5′) promoter sequence operably linked to a downstream (3′) nucleic acid sequence encoding a regulatory protein of the disclosure.

In related embodiments, recombinant filamentous fungal cells of the disclosure comprise one or more expression cassettes encoding one or more proteins of interest. For example, certain aspects are related to expression cassettes encoding proteins of interest, wherein the cassette comprises an upstream (5′) promoter operably linked to downstream (3′) nucleic acid sequence encoding the protein of interest. Certain other embodiments are therefore related to methods for producing increased amounts of a heterologous protein of interest (POI) in filamentous fungal host cells.

### II. Definitions

Prior to describing the present strains, compositions and methods in further detail, the following terms and phrases are defined. Terms not defined should be accorded their ordinary meaning as used and known to one skilled in the art.

All publications and patents cited in this specification are herein incorporated by reference.

Where a range of values is provided, it is understood that each intervening value, to the tenth of the unit of the lower limit unless the context clearly dictates otherwise, between the upper and lower limit of that range and any other stated or intervening value in that stated range, is encompassed within the present compositions and methods. The upper and lower limits of these smaller ranges may independently be included in the smaller ranges and are also encompassed within the present compositions and methods, subject to any specifically excluded limit in the stated range. Where the stated range includes one or both of the limits, ranges excluding either or both of those included limits are also included in the present compositions and methods.

Certain ranges are presented herein with numerical values being preceded by the term “about”. The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number. For example, in connection with a numerical value, the term “about” refers to a range of ⁻10% to ⁺10% of the numerical value, unless the term is otherwise specifically defined in context. In another example, the phrase a “pH value of about 6” refers to pH values of from 5.4 to 6.6, unless the pH value is specifically defined otherwise.

The headings provided herein are not limitations of the various aspects or embodiments of the present compositions and methods which can be had by reference to the specification as a whole. Accordingly, the terms defined immediately below are more fully defined by reference to the specification as a whole.

In accordance with this Detailed Description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

It is further noted that the claims may be drafted to exclude any optional element. As such, this statement is intended to serve as antecedent basis for use of such exclusive terminology as “solely”, “only”, “excluding”, “not including” and the like in connection with the recitation of claim elements, or use of a “negative” limitation or “proviso”. For example, in certain embodiments, the proviso “wherein the medium does not comprise an inducing substrate” may be used to exclude inducing substrates such as cellulose, lactose, gentibiose, sophorose and the like.

It is further noted that the term “comprising”, as used herein, means “including, but not limited to”, the component(s) after the term “comprising”. The component(s) after the term “comprising” are required or mandatory, but the composition comprising the component(s) may further include other non-mandatory or optional component(s).

It is also noted that the term “consisting of,” as used herein, means “including and limited to”, the component(s) after the term “consisting of”. The component(s) after the term “consisting of” are therefore required or mandatory, and no other component(s) are present in the composition.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

As used herein, the term “Ascomycete fungal cell” refers to any organism in the Division Ascomycota in the Kingdom Fungi. Examples of Ascomycetes fungal cells include, but are not limited to, filamentous fungi in the subphylum Pezizomycotina, such as Trichoderma sp., Aspergillus sp., Myceliophthora sp., Penicillium sp., and the like.

As used herein, the term “filamentous fungus” refers to all filamentous forms of the subdivision Eumycota and Oomycota. For example, filamentous fungi include, without limitation, Acremonium, Aspergillus, Emericella, Fusarium, Humicola, Mucor, Myceliophthora, Neurospora, Penicillium, Scytalidium, Thielavia, Tolypocladium, and Trichoderma species.

In certain embodiments, a filamentous fungus is a Trichoderma sp. cell (strain) including, but not limited to, Trichoderma harzianum, Trichoderma koningii, Trichoderma longibrachiatum, Trichoderma reesei and Trichoderma viride. As known to one skilled in the art, Trichoderma reesei was previously classified as “Hypocrea jecorina”.

In other embodiments, a filamentous fungus is an Aspergillus sp. cell (strain) such as Aspergillus aculeatus, Aspergillus awamori, Aspergillus clavatus, Aspergillus flavus, Aspergillus foetidus, Aspergillus fumigatus, Aspergillus japonicus, Aspergillus nidulans, Aspergillus niger, Aspergillus oryzae and Aspergillus terreus.

As used herein, the terms “wild-type” and “native” are used interchangeably and refer to genes, proteins, fungal cells or strains as found in nature.

As used herein, the terms “recombinant” or “non-natural” refer to an organism, microorganism, cell, nucleic acid molecule, or vector that has at least one engineered genetic alteration, or has been modified by the introduction of a heterologous nucleic acid molecule, or refer to a cell (e.g., a microbial cell) that has been altered such that the expression of a heterologous or endogenous nucleic acid molecule or gene can be controlled. Recombinant also refers to a cell that is derived from a non-natural cell or is progeny of a non-natural cell having one or more such modifications. Genetic alterations include, for example, modifications introducing expressible nucleic acid molecules encoding proteins, or other nucleic acid molecule additions, deletions, substitutions or other functional alteration of a cell's genetic material. For example, recombinant cells may express genes or other nucleic acid molecules that are not found in identical or homologous form within a native (wild-type) cell, or may provide an altered expression pattern of endogenous genes, such as being over-expressed, under-expressed, minimally expressed, or not expressed at all.

“Recombination”, “recombining” or generating a “recombined” nucleic acid is generally the assembly of two or more nucleic acid fragments wherein the assembly gives rise to a chimeric gene.

As used herein, the term “gene” is synonymous with the term “allele” in referring to a nucleic acid that encodes and directs the expression of a protein or RNA. Vegetative forms of filamentous fungi are generally haploid, therefore a single copy of a specified gene (i.e., a single allele) is sufficient to confer a specified phenotype.

As used herein, the term “gene” means the segment of DNA involved in producing a polypeptide (protein) chain, that may or may not include regions preceding and following the coding region (e.g., 5′ untranslated (5′ UTR) or “leader” sequences, 3′ UTR or “trailer” sequences, promoter sequences, terminator sequences and the like) as well as intervening sequences (introns) between individual coding segments (exons). For example, a gene (DNA) sequence of interest (GOI) may encode a regulatory protein, a structural protein, commercially important industrial proteins or peptides, such as enzymes (e.g., proteases, mannanases, xylanases, amylases, glucoamylases, cellulases, oxidases, phytases, lipases) and the like. The gene of interest may be a naturally occurring gene, a mutated (modified) gene or a synthetic gene.

As used herein, the term “promoter” refers to a nucleic acid sequence that functions to direct transcription of a downstream gene, or an open reading frame (ORF) thereof. The promoter will generally be appropriate to the host cell (e.g., a filamentous fungal cell) in which the target gene is being expressed. The promoter together with other transcriptional and translational regulatory nucleic acid sequences (also termed “control sequences”) is necessary to express a given gene. In general, the transcriptional and translational regulatory sequences include, but are not limited to, promoter and terminator sequences including a core promoter and enhancer or activator or repressor sequences, transcriptional and translational start and stop sequences. In certain embodiments, the promoter is an inducible promoter, or a constitutive promoter. In certain embodiments, the inducible promoter is an inducible cellulase gene promoter.

As used herein, the term “promoter activity” is the ability of a nucleic acid to direct transcription of a downstream (3′) polynucleotide in a host cell. To test promoter activity, the (promoter) nucleic acid may be operably linked to a downstream polynucleotide to produce a recombinant nucleic acid. The recombinant nucleic acid may be introduced into a cell, and transcription of the polynucleotide may be evaluated. In certain cases, the polynucleotide may encode a protein, and transcription of the polynucleotide can be evaluated by assessing production of the protein in the cell.

As used herein, the term “operably linked” refers to a functional linkage between two or more nucleic acid sequences. Thus, a nucleic acid sequence is operably linked when it is placed into a functional relationship with another nucleic acid sequence. For example, a promoter sequence or a terminator sequence is operably linked to a coding sequence if it affects the transcription of the coding sequence; a ribosome binding site is operably linked to a coding sequence if it is positioned so as to facilitate translation; a nucleic acid sequence encoding a secretory leader (i.e., a signal peptide) is operably linked to a nucleic acid sequence (e.g., an ORF) encoding a polypeptide if it is expressed as a pre-protein that participates in the secretion of the polypeptide. Generally, “operably linked” means that the DNA (nucleic acid) sequences being linked are contiguous, and, in the case of a secretory leader, contiguous and in reading phase. However, enhancers do not have to be contiguous. Linking two or more nucleic acid sequences (i.e., operably linking) is accomplished using any of the methods to one of skill in the art.

As used herein, exemplary parental Trichoderma reesei strains include, but are not limited to, T. reesei strain QM6a (ATCC® 13631), T. reesei strain RL-P37 (NRRL Deposit No. 15709) and T. reesei strain RUT-C30 (ATCC® 56765); exemplary parental Aspergillus niger strains include, but are not limited to, A. niger strain ATCC® 1015; exemplary parental Aspergillus oryzae strains include, but are not limited to A. oryzae strain RIB40 (ATCC® 42149); and exemplary parental Myceliophthora thermophila strains include, but are not limited to, M. thermophila strain ATCC® 42464.

For example, Trichoderma strains Rut-C30 and RL-P37 are mutagenized derivatives of Trichoderma natural isolate QM6a (Le Crom et al., 2009; Sheir-Neiss and Montenecourt, 1984), with strain NG14 being the last common ancestor. In certain aspects of the disclosure, an exemplary filamentous fungal strain is derived/obtained from T. reesei strain RL-P37 and comprises a deletion of the T. reesei pyr2 gene (abbreviated hereinafter, “Δpyr2”), as generally described by Sheir-Neiss and Montenecourt (1984) and PCT Publication No. WO2011/153449.

As used herein, the phrases “lignocellulosic degrading enzymes”, “cellulase enzymes”, and “cellulases” are used interchangeably, and include glycoside hydrolase (GH) enzymes such as cellobiohydrolases, xylanases, endoglucanases, and β-glucosidases, that hydrolyze glycosidic bonds of cellulose (hemi-cellulose) to produce sugars (e.g., glucose, xylose, arabinose, etc.).

As used herein, “endoglucanase” proteins may be abbreviated as “EG”, “cellobiohydrolase” proteins may be abbreviated “CBH”, “β-glucosidase” proteins may be abbreviated “BG” and “xylanase” proteins may be abbreviated “XYL”. Thus, as used herein, a gene (or ORF) encoding a EG protein may be abbreviated “eg”, a gene (or ORF) encoding a CBH protein may be abbreviated “cbh”, a gene (or ORF) encoding a BG protein may be abbreviated “bg”, and a gene (or ORF) encoding a XYL protein may be abbreviated “xyl”. In certain embodiments, cellobiohydrolases include enzymes classified under Enzyme Commission No. (EC 3.2.1.91), endoglucanases include enzymes classified under EC 3.2.1.4, endo-β-1,4-xylanases include enzymes classified under EC 3.2.1.8, β-xylosidases include enzymes classified under EC 3.2.1.37, and β-glucosidases include enzymes classified under EC 3.2.1.21.

As used herein, a “cellulase gene promoter” includes, but is not limited to, a cellobiohydrolase (cbh) gene promoter sequence, an endoglucanase (eg) gene promoter sequence, a β-glucosidase (bg) gene promoter sequence, a xylanase (xyl) gene promoter sequence, and the like.

As used herein, the terms “modification” and “genetic modification” are used interchangeably and include: (a) the introduction, substitution, or removal of one or more nucleotides in a gene, or the introduction, substitution, or removal of one or more nucleotides in a regulatory element required for the transcription or translation of the gene, (b) a gene disruption, (c) a gene conversion, (d) a gene deletion, (e) the down-regulation and/or up-regulation of a gene, (f) specific mutagenesis and/or (g) random mutagenesis of any one or more the genes/DNA sequences disclosed herein.

As used herein, the phrases “modified filamentous fungal cell(s)”, “mutant filamentous fungal cell(s)”, “recombinant fungal cell(s)”, “modified filamentous fungal strain(s)”, and the like may be used interchangeably and refer to filamentous fungal cells that are derived (i.e., obtained) from a parental filamentous fungal cell belonging to the Pezizomycotina subphylum. For example, a “modified” filamentous fungal cell may be derived (obtained) from a parental filamentous fungal cell, wherein the modified cell comprises at least one genetic modification which is not found in the parental cell.

As used herein, an exemplary phytase (reporter) protein is the engineered Buttiauxella sp. phytase protein described in PCT Publication No. WO2008097619A2 (incorporated herein by reference in its entirety), and an exemplary amylase (reporter) protein is the alpha-amylase protein from Aspergillus terreus (NCBI Accession Nr. XP_001209405).

As used herein, the term “amylase” refers to a glycoside hydrolase (enzyme) that is, among other things, capable of catalyzing the degradation of starch. Such amylase enzymes include, but are not limited to, endo-acting α-amylases (EC 3.2.1.1; α-D-(1→4)-glucan glucanohydrolase), exo-acting β-amylases (EC 3.2.1.2; α-D-(1→4)-glucan maltohydrolase) and product-specific amylases, such as maltogenic α-amylase (EC 3.2.1.133), α-glucosidases (EC 3.2.1.20; α-D-glucoside glucohydrolase), glucoamylase (EC 3.2.1.3; α-D-(1→4)-glucan glucohydrolase), maltotetraosidases (EC 3.2.1.60), maltohexaosidases (EC 3.2.1.98) and the like.

As generally understood by one of skill in the art, such amylases are particularly suitable for use in starch liquefaction and saccharification, cleaning starchy stains, textile de-sizing, baking, brewing and the like.

As used herein, the term “phytase” refers to an enzyme that is, among other things, capable of catalyzing the hydrolysis of phytic acid and releasing inorganic phosphorus. As generally understood by one of skill in the art, phytases are particularly suitable for use in animal feed and the like.

As used herein, a “functional gene” is a gene capable of being used by cellular components to produce an active gene product, typically a protein. In contrast, a “non-functional gene” cannot be used by cellular components to produce an active gene product (i.e., a functional protein), or has a reduced ability to be used by cellular components to produce an active gene product (i.e., a functional protein).

As used herein, a “functional protein” is a protein that possesses a function or activity, such as an enzymatic function/activity, a binding function/activity (e.g., DNA binding), a surface-active property, and the like, and which has not been mutagenized, truncated, or otherwise modified to abolish or reduce that function/activity.

As used herein, a “regulatory gene” is a gene whose function has an effect on production of proteins by the fungal host. In certain aspects of the disclosure, the overexpression of a regulatory gene (encoding a regulatory protein) has an effect on protein production by the filamentous fungal (host) cells. A “regulatory gene” encodes a “regulatory protein”.

As used herein, a “regulatory protein” includes, but is not limited to, transcription factor proteins, protein kinases, proteins involved in histone modification or chromatin remodeling, and other “gene transcription regulatory proteins”.

More particularly, as used herein, the terms “regulatory gene(s)” “regulatory protein(s)” and/or other “gene transcription regulatory proteins” are not meant to be limiting, but rather used herein to help classify, characterize and/or identify the exemplary genes and/or proteins of the instant disclosure. Thus, as further specified below in Section III, the Examples that follow (and TABLES 1-6 therein), the DNA sequences (e.g., TABLE 1) and/or proteins sequence are particularly referred to herein as regulatory genes and/or proteins, regardless of overall protein function. For example, a regulatory protein set forth in TABLES 1-6 may include proteins (e.g., enzymes) involved in one or more metabolic pathway activities (e.g., such as to alleviate a pathway bottleneck) and the like, wherein such proteins (enzymes) may be referred to herein as regulatory proteins, regardless of overall protein function.

As further detailed and described below, certain aspects of the disclosure are related to the overexpression of certain regulatory genes encoding regulatory proteins As used herein, the “overexpression” (abbreviated, “OE”) of a gene encoding a regulatory protein can be carried out, for example, by introducing into a fungal host an additional copy (or copies) of a specific gene encoding a regulatory protein (e.g., see, Example 1; TABLE 1), or by expressing the gene encoding a regulatory protein under the control of a heterologous promoter resulting in increased expression of the gene, or otherwise genetically modifying the fungal host so that either the gene is more abundantly expressed or the activity of the gene product is increased. The effect of overexpression of a gene encoding a regulatory protein can be studied by culturing the modified host under conditions suitable for protein production. The effect on the production of an endogenous protein or heterologous protein can be studied by determining for example a specific enzyme activity, determining the amount of total protein, or determining the amount of specific endogenous or heterologous protein produced.

As used herein, “disruption of a gene”, “gene disruption”, “inactivation of a gene” and “gene inactivation” are used interchangeably and refer broadly to any genetic modification that substantially prevents a host cell from producing a functional gene product (e.g., a functional protein). Exemplary methods of gene disruptions include complete or partial deletion of any portion of a gene, including a polypeptide-coding sequence, a promoter, an enhancer, or another regulatory element, or mutagenesis of the same, where mutagenesis encompasses substitutions, insertions, deletions, inversions, and any combinations and variations thereof which disrupt/inactivate the target gene(s) and substantially reduce or prevent the production of the functional gene product (i.e., the functional protein).

As used herein, “deletion of a gene,” refers to its removal from the genome of a host cell. Where a gene includes control elements (e.g., enhancer elements) that are not located immediately adjacent to the coding sequence of a gene, deletion of a gene refers to the deletion of the coding sequence, and optionally adjacent enhancer elements, including but not limited to, for example, promoter and/or terminator sequences.

As used herein, a “heterologous gene” refers to polynucleotide (DNA) sequences having at least a portion of the sequence which is not native or existing in a native form to the cell in which it is introduced and/or expressed.

As used herein, a “heterologous nucleic acid construct” or “heterologous DNA sequence” has a portion of the sequence which is not native or existing in a native form to the cell in which it is expressed.

As used herein, a “heterologous protein” is encoded by a heterologous gene, a heterologous nucleic acid (polynucleotide) sequence, a heterologous DNA sequence, and the like.

Thus, in certain embodiments, a heterologous gene, a heterologous nucleic acid construct, a heterologous DNA sequence, etc. encoding a protein of interest (POI) is introduced (e.g., transformed) into a filamentous fungal cell (strain). For example, a heterologous gene construct encoding a POI may be introduced into the filamentous fungal cell (strain) before, during, or after performing other genetic modification described herein.

Heterologous, with respect to a control sequence refers to a control sequence (e.g., promoters, enhancers, terminators) that does not function in nature to regulate the same gene the expression of which it is currently regulating. Generally, heterologous nucleic acid sequences are not endogenous to the cell or part of the genome in which they are present, and have been added to the cell, by infection, transfection, transformation, microinjection, electroporation, or the like. A “heterologous” nucleic acid construct may contain a control sequence/DNA coding sequence combination that is the same as, or different from a control sequence/DNA coding sequence combination found in the native cell.

As used herein, the term “coding sequence” refers to a polynucleotide sequence, which directly specifies the amino acid sequence of its (encoded) protein product. The boundaries of the coding sequence are generally determined by an open reading frame (ORF), which usually begins with a start codon (ATG). The coding sequence typically includes DNA, cDNA, and recombinant nucleotide sequences. For example, an ORF generally refers to polynucleotide sequence (whether naturally occurring, non-naturally occurring, or synthetic) comprising an uninterrupted reading frame consisting of (i) an initiation codon, (ii) a series of codons representing amino acids of the encoded protein product, and (iii) a termination codon, the ORF being read (or translated) in the 5′ to 3′ direction.

As used herein, the term “DNA construct” or “expression construct” refers to a nucleic acid sequence, which comprises at least two DNA polynucleotide fragments. A DNA or expression construct can be used to introduce nucleic acid sequences into a fungal host cell. The DNA may be generated in vitro (e.g., by PCR) or any other suitable techniques. In some preferred embodiments, the DNA construct comprises a sequence of interest (e.g., encoding a protein of interest). In certain embodiments, a polynucleotide sequence of interest is operably linked to a promoter and/or terminator. In some embodiments, the DNA construct further comprises at least one selectable marker. In further embodiments, the DNA construct comprises sequences homologous to the host cell chromosome. In other embodiments, the DNA construct comprises non-homologous sequences to the host cell chromosome.

As used herein, a “flanking sequence” refers to any sequence that is either upstream or downstream of the sequence being discussed (e.g., for genes A-B-C, gene B is flanked by the A and C gene sequences). In certain embodiments, the incoming sequence is flanked by a homology box on each side. In another embodiment, the incoming sequence and the homology boxes comprise a unit that is flanked by stuffer sequence on each side. In some embodiments, a flanking sequence is present on only a single side (either 3′ or 5′), but in preferred embodiments, it is on each side of the sequence being flanked. The sequence of each homology box is homologous to a sequence in the filamentous fungal chromosome. These sequences direct where in the filamentous fungal chromosome the new construct gets integrated and what part of the chromosome will be replaced by the incoming sequence.

As used herein, the term “down-regulation” of gene expression includes any methods that result in lower (down-regulated) expression of a functional gene product (i.e., the functional protein).

The term “vector” is defined herein as a polynucleotide designed to carry nucleic acid sequences to be introduced into one or more cell types. Vectors include cloning vectors, expression vectors, shuttle vectors, plasmids, phage or virus particles, DNA constructs, cassettes and the like. Expression vectors may include regulatory sequences such as promoters, signal sequences, a coding sequences and transcription terminators.

An “expression vector” as used herein means a DNA construct comprising a coding sequence that is operably linked to suitable control sequences capable of effecting expression of a protein in a suitable host. Such control sequences may include a promoter to effect transcription, an optional operator sequence to control transcription, a sequence encoding suitable ribosome binding sites on the mRNA, enhancers and sequences which control termination of transcription and translation.

As used herein, the term “secretory signal sequence” denotes a DNA sequence that encodes a polypeptide (i.e., a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

As used herein, the term “isolated” or “purified” refers to a filamentous fungal cell, a nucleic acid or a polypeptide that is removed from at least one component with which it is naturally associated.

As used herein, the terms “protein of interest” or “POI” refer to a polypeptide that is desired to be expressed in a filamentous fungal cell. Such a protein can be an enzyme, a substrate-binding protein, a surface-active protein, a structural protein, and the like, and can be expressed at high levels, and can be for the purpose of commercialization. For example, as generally set forth below, a protein of interest (POI) includes, but is not limited to, cellulases, hemicellulases, xylanases, peroxidases, proteases, lipases, phospholipases, esterases, cutinases, polyesterases, pectinases, keratinases, reductases, oxidases, phenol oxidases, lipoxygenases, ligninases, pullulanases, tannases, pentosanases, mannanases, α-glucanases, β-glucanases, hyaluronidases, chondroitinases, laccases, amylases, glucoamylases, acetyl esterases, aminopeptidase, arabinases, arabinosidases, arabinofuranosidases, carboxypeptidases, catalases, nucleases, deoxyribonucleases, ribonucleases, epimerases, α-galactosidases, β-galactosidases, glucan lysases, endo-β-glucanases, glucose oxidases, glucuronidases, invertases, isomerases, and the like.

A protein of interest (POI) can be encoded by an “endogenous” gene. For example, in certain embodiments, a protein of interest (POI) is encoded by a gene endogenous to the filamentous fungal cell (strain), such as the aforementioned wild-type genes encoding the native suite of cellulases (e.g., cellobiohydrolases, xylanases, endoglucanases and β-glucosidases).

As used herein, the term “increased productivity” and variations thereof mean an increase of at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20% (e.g., greater than 20%) in the production of a protein of interest by a modified (mutant) filamentous fungal cell overexpressing a regulatory gene encoding a regulatory protein of the disclosure, when cultivated under the same conditions of medium composition, temperature, pH, cell density, dissolved oxygen, and time as the parent (control) filamentous fungal cell which does not overexpress the regulatory protein.

As used herein, the term “increased amount” when used in phrases such as a recombinant cell “produces an ‘increased amount’ of a protein of interest”, and variations thereof mean an increase of at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20% (e.g., greater than 20%) in the amount of a protein of interest produced by a modified (mutant) filamentous fungal cell overexpressing a regulatory gene encoding a regulatory protein of the disclosure, when cultivated under the same conditions of medium composition, temperature, pH, cell density, dissolved oxygen, and time as the parent (control) filamentous fungal cell which does not overexpress the regulatory protein.

As used herein, the terms “polypeptide” and “protein” (and/or their respective plural forms) are used interchangeably to refer to polymers of any length comprising amino acid residues linked by peptide bonds. The conventional one-letter or three-letter codes for amino acid residues are used herein. The polymer can be linear or branched, it can comprise modified amino acids, and it can be interrupted by non-amino acids. The terms also encompass an amino acid polymer that has been modified naturally or by intervention (e.g., disulfide bond formation, glycosylation, lipidation, acetylation, phosphorylation, or any other manipulation or modification, such as conjugation with a labeling component). Also included within the definition are, for example, polypeptides containing one or more analogs of an amino acid (including, for example, unnatural amino acids, etc.), as well as other modifications known in the art.

As used herein, functionally and/or structurally similar proteins are considered to be “related proteins.” Such proteins can be derived from organisms of different genera and/or species, or even different classes of organisms (e.g., bacteria and fungi). Related proteins also encompass homologs determined by primary sequence analysis, determined by secondary or tertiary structure analysis, or determined by immunological cross-reactivity.

As used herein, the phrase “substantially free of an activity,” or similar phrases, means that a specified activity is either undetectable in an admixture or present in an amount that would not interfere with the intended purpose of the admixture.

As used herein, the term “derivative polypeptide” refers to a protein which is derived or derivable from a protein by addition of one or more amino acids to either or both the N- and C-terminal end(s), substitution of one or more amino acids at one or a number of different sites in the amino acid sequence, deletion of one or more amino acids at either or both ends of the protein or at one or more sites in the amino acid sequence, and/or insertion of one or more amino acids at one or more sites in the amino acid sequence. The preparation of a protein derivative can be achieved by modifying a DNA sequence which encodes for the native protein, transformation of that DNA sequence into a suitable host, and expression of the modified DNA sequence to form the derivative protein.

Related (and derivative) proteins include “variant proteins.” Variant proteins differ from a reference/parental protein (e.g., a wild-type protein) by substitutions, deletions, and/or insertions at a small number of amino acid residues. The number of differing amino acid residues between the variant and parental protein can be one or more, for example, 1, 2, 3, 4, 5, 6, 7, 8, 9, 10, 15, 20, 30, 40, 50, or more amino acid residues. Variant proteins can share at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99%, or more, amino acid sequence identity with a reference protein. A variant protein can also differ from a reference protein in selected motifs, domains, epitopes, conserved regions, and the like.

As used herein, the term “homologous” protein refers to a protein that has similar activity, function and/or structure to a reference protein. It is not intended that homologs necessarily be evolutionarily related. Thus, it is intended that the term encompass the same, similar, or corresponding protein(s) (i.e., in terms of structure and function) obtained from different organisms. In some embodiments, it is desirable to identify a homolog that has a quaternary, tertiary and/or primary structure similar to the reference protein. For example, regulatory protein homologues from Aspergillus niger, Aspergillus oryzae and/or Thermothelomyces thermophilus (i.e., comprising substantial amino acid sequence identity to a full-length T. reesei regulatory protein of the disclosure) are described in Example 10 (see, TABLE 6).

The degree of homology between sequences can be determined using any suitable method known in the art (see, e.g., Smith and Waterman, 1981; Needleman and Wunsch, 1970; Pearson and Lipman, 1988; programs such as GAP, BESTFIT, FASTA, and TFASTA in the Wisconsin Genetics Software Package (Genetics Computer Group, Madison, WI); and Devereux et al., 1984). For purposes of the present invention, the degree of identity between two amino acid sequences is determined using the Needleman-Wunsch algorithm (Needleman and Wunsch, 1970) as implemented in the Needle program of the EMBOSS package (Rice et al., 2000), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the degree of identity between two deoxyribonucleotide sequences is determined using the Needleman-Wunsch algorithm (Needleman and Wunsch, 1970, supra) as implemented in the Needle program of the EMBOSS package (Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment)

As used herein, the phrases “substantially similar” and “substantially identical”, in the context of at least two nucleic acids or polypeptides, typically means that a polynucleotide or polypeptide comprises a sequence that has at least about 40% identity, at least about 50% identity, at least about 60% identity, at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Sequence identity can be determined using known programs such as BLAST, ALIGN, and CLUSTAL using standard parameters.

As used herein, the terms “inducer”, “inducers”, or “inducing substrates” are used interchangeably and refer to any compounds that cause filamentous fungal cells to produce “increased amounts” of total protein. Examples of inducing substrates include, but are not limited to, sophorose, lactose, gentibiose and cellulose.

As used herein, the term “induction” refers to the increased transcription of a gene resulting in the synthesis of a protein of interest (POI) in a filamentous fungal cell at a markedly increased rate in response to the presence of the “inducer” (i.e., inducing substrate).

To measure the “induction” of a gene of interest (GOI), encoding a protein of interest (POI), modified filamentous fungal cells are treated with a candidate inducing substrate (inducer) and are compared vis-à-vis to parental filamentous fungal (control) cells which are not treated with the inducing substrate (inducer). Thus, the untreated parental (control) cells are assigned a relative protein activity value of 100%, wherein induction of the GOI encoding the POI in the modified host cells is achieved when the activity value (i.e., relative to the control cells) is greater than 100%, greater than 105%, greater than 110%, greater than 150%, greater than 200-500% (i.e., relative to the control), or higher.

As used herein, “aerobic fermentation” refers to growth in the presence of oxygen.

As used herein, the term “cell broth” refers collectively to medium and cells in a liquid/submerged culture.

As used herein, the term “cell mass” refers to the cell component (including intact and lysed cells) present in a liquid/submerged culture. Cell mass can be expressed in dry or wet weight.

As used herein, the phrase “elevated fermentation (cultivation) temperature” is a fermentation temperature greater than the standard fermentation conditions described in the Examples

As used herein, the term “sporulation” when used in phrases such as “improved sporulation”, “enhanced sporulation”, “increased sporulation”, “improved sporulation phenotype” and the like means higher amount of spores formed by a fungal strain grown on an appropriate nutrient medium, either on agar plates or in liquid cultures, as compared to a reference strain. For example, in certain embodiments, recombinant fungal strains overexpressing a regulatory protein comprising at least 80% identity to SEQ ID NO: 68 demonstrate improved sporulation phenotypes relative to control strains which do not overexpress any regulatory proteins.

It will be understood that the methods of the present disclosure are not limited to a particular order for obtaining the modified (mutant) filamentous fungal cell (strain). The modification of a gene may be introduced into the parent strain at any step in the construction of the strain for the production of an endogenous protein of interest (POI) and/or the production of a heterologous POI.

### III. Regulatory Proteins

As generally understood by one skilled in the art, regulatory proteins are involved in the regulation of cell homeostasis at different levels, including, but not limited to, the process of transcribing DNA into RNA. For example, many regulatory proteins called transcription factors, are typically sequence-specific DNA-binding proteins that control, regulate, mediate, and the like, the rate of gene transcription by binding to specific (target) sites in the promoter regions of the (regulated) genes (Latchman, 1993). More particularly, one distinct feature of transcription factors is that they have DNA-binding domains (DBDs) that give them the ability to bind to specific sequences of DNA called enhancer or promoter sequences. Thus, some regulatory proteins bind to a DNA promoter sequence near the transcription start site and help to form the transcription initiation complex. Other regulatory proteins bind to regulatory sequences, such as enhancer sequences, and can either stimulate or repress transcription of the related gene, wherein these regulatory sequences can be thousands of base pairs (bp) upstream (5′) or downstream (3) from the gene being transcribed.

Protein kinases are regulatory proteins that can selectively modify other proteins by (covalently) adding phosphates to them (i.e., phosphorylation). Histone acetylases/deacetylases are regulatory proteins that can modify histone proteins via covalent addition/removal of acetyl groups (COCH₃), respectively. For example, the regulation of transcription is the most common form of gene control, wherein the action of regulatory proteins allows for unique spatiotemporal regulation of gene expression patterns.

The production of cellulases, hemi-cellulases, ligninases, pectinases and the like are believed to be mainly regulated at the transcriptional level in filamentous fungi (Aro et al., 2005). For example, Stricker et al. (2008) described the similarities and differences in the transcriptional regulation of expression of cellulases and hemi-cellulases in Aspergillus niger and Trichoderma reesei, including the action of XlnR and Xyr1. As described by Kubicek et al. (2009), in T. reesei some regulatory components function in cellulase regulation positively (Xyr1, Ace2, Hap2/3/5) and some negatively (Ace1, Cre1). Although the action of some regulatory genes on the production of proteins have been described, there is still a need for improved filamentous fungal strains capable of enhanced production of endogenous and heterologous proteins of interest.

As generally known in the art, filamentous fungal strains are typically grown as mycelial submerged cultures in bioreactors, which are adapted to introduce and distribute oxygen and nutrients into the culture medium (i.e., fermentation broth) and maintain optimal pH and temperature, among other things. In particular, the power required to mix, aerate, cool, etc. the fermentation broth can significantly increase the cost of production, and incur higher capital expenditures in terms of motors, power supplies, cooling equipment and the like. For example, the evolution of heat during fermentation processes is closely related to the utilization of carbon and energy source (Wang et al., 1979). As generally described by Wang et al. (1979), the amount of heat is related to the stoichiometry for growth and product formation, while the rate of heat evolution is proportional to kinetics of the process, wherein interest in heat evolution stems from the need to remove it during the fermentation process (e.g., to maintain optimal product formation). Thus, running the process at a higher temperature may be beneficial as it can reduce fermentation time and releases fermentation capacities.

In general, most filamentous fungi will only grow and efficiently produce within a temperature range of about 20-40° C. For example, the fermentation temperature can vary somewhat, but for filamentous fungi such as Trichoderma reesei, the temperature generally will be within the range of about 20° C. to 40° C., generally preferably in the range of about 25° C. to 34° C. Thus, the ability to ferment filamentous fungal strains at elevated temperatures for the production of proteins is of particular interest in reducing the costs of protein production. As described hereinafter, the recombinant filamentous fungal cells (strains) of the disclosure are well-suited for use in industrial scale fermentation processes for the enhanced production of proteins of interest.

More particularly, as described herein and set forth below in the Examples section, Applicant has identified and screened more than seven-hundred fifty (750) regulatory proteins for their ability to enhance the protein production in filamentous fungal (host) cells. More specifically, as described in Examples, Applicant has designed/constructed exemplary T. reesei reporter strains and modified (recombinant) strains thereof overexpressing a regulatory protein (e.g., see TABLE 1, Example 1) or a combination of two regulatory proteins thereof (TABLES 4 and 5). For example, the T. reesei reporter strain described in Example 1C was constructed to express two exemplary reporter proteins (i.e., a Buttiauxella sp. phytase and an A. terreus alpha-amylase); wherein the T. reesei reporter strain expressing the two reporter proteins was further modified to overexpress a single regulatory protein (Example 1D) or combination of two regulatory proteins (Example 1E).

As set forth in Examples 4, the overexpression of six-hundred ninety-one (691) single regulatory proteins were screened for improved protein production under various conditions, wherein the overexpression of the regulatory proteins set forth in TABLE 2 resulted in increased production of one, or both reporter proteins, when fermented under standard conditions in lactose releasing plates after one-hundred twenty (120) hours of incubation. In addition, as set forth in Example 5, the overexpression of the regulatory proteins set forth in TABLE 3 resulted in increased production of one, or both reporter proteins, when fermented at elevated temperatures in lactose releasing plates after one-hundred twenty (120) hours of incubation

As further described in Example 6, one-hundred thirty-four (134) recombinant strains each overexpressing two different regulatory proteins, or two of the same regulatory proteins, were screened for improved protein production under various conditions. For example, as set forth in TABLE 4, the mutant (recombinant) cells simultaneously overexpressing two T. reesei regulatory gene (DNA) sequence combinations demonstrate enhanced protein productivity phenotypes (i.e., relative to the parental/control cells) when fermented under lactose releasing conditions.

In addition, as set forth in TABLE 5, the recombinant cells simultaneously overexpressing two T. reesei regulatory gene (DNA) sequence combinations demonstrate enhanced protein productivity phenotype (i.e., relative to the parental/control cells) when fermented with 2% (w/w) glucose/sophorose as a carbon source.

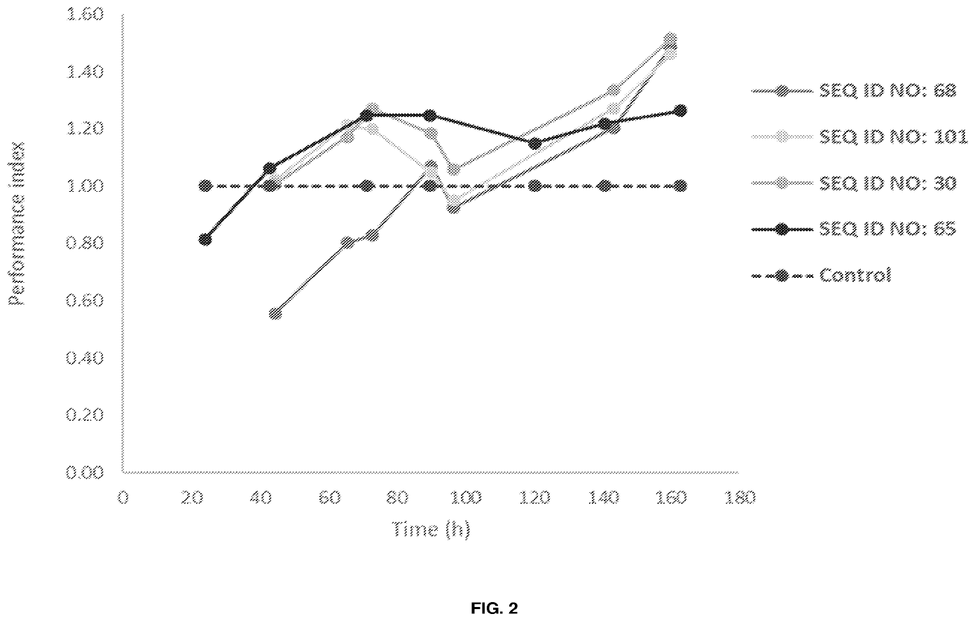

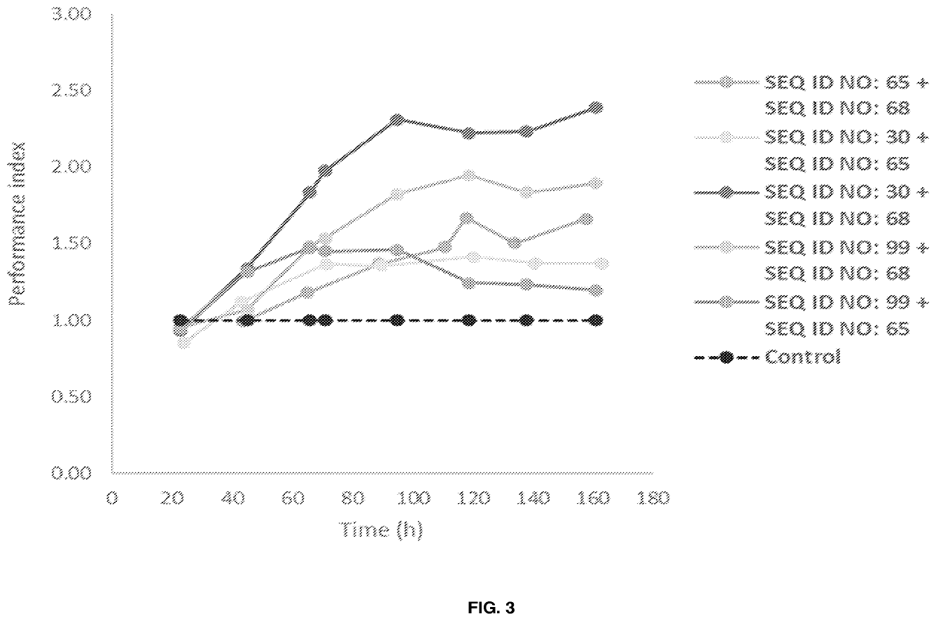

Likewise, as generally described in Examples 7-9, a subset of T. reesei strains overexpressing a single regulatory protein (Examples 8) or two regulatory proteins (Example 9) were evaluated in two (2) L bioreactors (in a fed-batch fermentation), using sophorose as an inducing substrate at a constant feed rate. More particularly, as described in Example 8, all of the T. reesei mutant strains overexpressing a regulatory protein of SEQ ID NO: 31, SEQ ID NO: 66, SEQ ID NO: 69, or SEQ ID NO: 102 (see, FIG. 2) exhibited improved protein production relative to the control strain. In addition, as described in Example 9, T. reesei mutant (recombinant) strains overexpressing a regulatory protein combination of SEQ ID NO: 66 and 69, SEQ ID NO: 31 and 66, SEQ ID NO: 31 and 69, SEQ ID NO: 100 and 69, or SEQ ID NO: 100 and 66 (see, FIG. 3), all exhibited higher protein productivity than the control strain throughout the fermentation.

As described in Example 10, filamentous fungal regulatory genes encoding regulatory proteins homologous to a T. reesei regulatory protein are presented in TABLE 6. Thus, in certain aspects, the disclosure is related to recombinant filamentous fungal cells overexpressing a regulatory gene comprising at least 80% identity to a Trichoderma reesei gene of SEQ ID NO: 1, 4, 7, 10, 12, 15, 18, 21, 23, 25, 28, 30, 33, 36, 39, 42, 45, 48, 51, 54, 56, 59, 62, 65, 68, 71, 74, 77, 79, 82, 84, 87, 90, 94, 97, 99, 101, 105, 108, 110, 113, 116, 119, 122, 125, 128 or 131, 134, 137, 140, 144, 147, 149, 152, and/or recombinant filamentous fungal cells overexpressing a regulatory gene comprising at least 80% identity to an orthologous gene sequence from a filamentous fungal cell shown in TABLE 6. Certain other embodiments are therefore related to recombinant filamentous fungal cells overexpressing a regulatory protein comprising at least 80% identity to a regulatory protein of SEQ ID NO: 2, 5, 8, 11, 13, 16, 19, 22, 24, 26, 29, 31, 34, 37, 40, 43, 46, 49, 52, 55, 57, 60, 63, 66, 69, 72, 75, 78, 80, 83, 85, 88, 91, 95, 98, 100, 102, 106, 109, 111, 114, 117, 120, 123, 126, 129 or 132, 135, 138, 141, 145, 148, 150, 153, and/or overexpressing a regulatory protein comprising at least 80% identity to a regulatory protein ortholog from a filamentous fungal cell shown in TABLE 6. Certain other embodiments provide recombinant filamentous fungal cells overexpressing a combination of at least two regulatory genes comprising at least 80% identity to a gene sequence of combination set forth in TABLE 4 and/or TABLE 5. Certain other embodiments provide recombinant filamentous fungal cells overexpressing a combination of at least two regulatory proteins comprising at least 80% identity to a regulatory protein combination set forth in TABLE 4 and/or TABLE 5.

As further described below, certain embodiments are related to polynucleotide constructs (e.g., expression cassettes) encoding regulatory proteins of the disclosure (see, Example 1; TABLE 1). In certain embodiments, a polynucleotide construct comprises an upstream (5′) promoter sequence operably linked to a downstream (3′) nucleic acid sequence encoding a regulatory protein comprising at least 80% sequence identity to a regulatory protein of SEQ ID NO: 2, 5, 8, 11, 13, 16, 19, 22, 24, 26, 29, 31, 34, 37, 40, 43, 46, 49, 52, 55, 57, 60, 63, 66, 69, 72, 75, 78, 80, 83, 85, 88, 91, 95, 98, 100, 102, 106, 109, 111, 114, 117, 120, 123, 126, 129 132, 135, 138, 141, 145, 148, 150 or 153. Thus, certain other embodiments are related to recombinant filamentous fungal cells comprising expression cassettes encoding such regulatory proteins. Certain other embodiments are directed to methods for producing increased amount of proteins of interest in filamentous fungal cells.

### IV. Recombinant Nucleic Acids and Molecular Biology

Thus, as generally set forth above and further exemplified below, certain embodiments of the disclosure are related to genetically modified filamentous fungal cells/strains overexpressing one or more (regulatory) genes encoding regulatory proteins of the disclosure (e.g., see Example 1; TABLE 1). Thus, in certain aspects, a filamentous fungal cell comprises one or more (multiple) introduced polynucleotides. In certain embodiments, an introduced polynucleotide is an expression cassette comprising in the 5′ to 3′ direction, an upstream (5′) promoter sequence operably linked to a downstream (3′) gene (or open reading frame; ORF) sequence encoding a protein of interest (POI). In related embodiments, the cassette may further comprise a downstream (3′) terminator sequence operably linked to the gene or ORF encoding the POI.

In certain other embodiments, an introduced polynucleotide is an expression cassette comprising in the 5′ to 3′ direction, an upstream promoter sequence operably linked to a downstream regulatory gene sequence (i.e., a encoding a regulatory protein). In related embodiments, the cassette may further comprise a downstream (3′) terminator sequence operably linked to the regulatory gene encoding the regulatory protein.

For example, a regulatory gene expression cassette comprising an upstream promoter operably linked to a regulatory gene (encoding a regulatory protein) is schematically presented below (Scheme A), wherein the promoter [pro] and regulatory gene [reg_gene] sequences are shown in operable “-” combination in the 5′ to 3′ direction:
Scheme A: 5′-[pro]-[reg_gene]-3′

Likewise, a regulatory gene expression cassette comprising an upstream promoter operably linked to a regulatory gene (encoding a regulatory protein) operably linked to a downstream terminator sequence is schematically presented below in Scheme B, wherein the promoter [pro], regulatory gene [reg_gene] and terminator [term] sequences are shown in operable “-” combination in the 5′ to 3′ direction.
Scheme B: 5′-[pro]-[reg_gene]-[term]-3′

As presented in Schemes A or B, the promoter and/or terminator sequences are not meant to be limiting, but are rather selected so as to be functional in the desired fungal cell/strain. For example, a promoter sequence can be any nucleotide sequence that shows transcriptional activity in the filamentous fungal cell, including mutant/variant promoters, truncated promoters, tandem promoters, hybrid promoters, synthetic promoters, inducible promoters, tuned promoters, conditional expression systems and combinations thereof. Often, suitable promoters can be obtained from genes encoding extracellular or intracellular polypeptides either native or heterologous (foreign) to the filamentous fungal cell. Examples of promoters suitable for driving the expression of one or more regulatory genes of the disclosure include, but are not limited, to a Trichoderma reesei cDNA1 promoter, an enol promoter, a pdc1 promoter, a pki1 promoter, a tef1 promoter, a rp² promoter, and other T. reesei promoters described in Fitz et al. 2018 (incorporated herein by referenced in its entirety), the Aspergillus oryzae thiA promoter, the A. nidulans gpdA promoter, and the like.

Thus, a nucleic acid construct comprising a polynucleotide encoding a regulatory protein (or a protein of interest) can be operably linked to one or more control sequences (e.g., a promoter sequence, a terminator sequence) capable of directing expression of the coding sequence in a filamentous fungal cell of the disclosure, e.g., under conditions compatible with the control sequences.

As set forth below in the examples, recombinant filamentous fungal cells of the disclosure may be constructed using routine methods well known in the art (e.g., Ausubel et al., 2003).

In certain embodiments, a modified (mutant) filamentous fungal cell may be constructed via CRISPR-Cas9 editing. For example, a gene of interest can be modified, disrupted, deleted, or down-regulated by means of nucleic acid guided endonucleases, that find their target DNA by binding either a guide RNA (e.g., Cas9 and Cpf1) or a guide DNA (e.g., NgAgo), which recruits the endonuclease to the target sequence on the DNA, wherein the endonuclease can generate a single or double stranded break in the DNA. This targeted DNA break becomes a substrate for DNA repair, and can recombine with a provided editing template to disrupt or delete or modify the gene. For example, the gene encoding the nucleic acid guided endonuclease (for this purpose Cas9 from S. pyogenes) or a codon optimized gene encoding the Cas9 nuclease is operably linked to a promoter active in the fungal cell and a terminator active in a fungal cell, thereby creating a fungal Cas9 expression cassette. Likewise, one or more target sites unique to the gene of interest are readily identified by a person skilled in the art. For example, to build a DNA construct encoding a gRNA-directed to a target site within the gene of interest, the variable targeting domain (VT) will comprise nucleotides of the target site which are 5′ of the (PAM) protospacer adjacent motif (NGG), which nucleotides are fused to DNA encoding the Cas9 endonuclease recognition domain for S. pyogenes Cas9 (CER). The combination of the DNA encoding a VT domain and the DNA encoding the CER domain thereby generate a DNA encoding a gRNA. Thus, a fungal cell expression cassette for the gRNA is created by operably linking the DNA encoding the gRNA to a promoter active in fungal cell and a terminator active in fungal cell.

In certain embodiments, the DNA break induced by the endonuclease is repaired/replaced with an incoming sequence. For example, to precisely repair the DNA break generated by the Cas9 expression cassette and the gRNA expression cassette described above, a nucleotide editing template is provided, such that the DNA repair machinery of the cell can utilize the editing template. For example, about 500 bp 5′ of the targeted gene can be fused to about 500 bp 3′ of the targeted gene to generate an editing template, which template is used by the fungal host's machinery to repair the DNA break generated by the RNA-guided endonuclease (RGEN). Even shorter stretches of nucleotides in a form of double or single stranded DNA can be used as an editing template.

The Cas9 expression cassette, the gRNA expression cassette and the editing template can be co-delivered to filamentous fungal cells using many different methods (e.g., protoplast fusion, electroporation, natural competence, or induced competence). The transformed cells are screened by PCR amplifying the target gene with a forward and reverse primer. These primers can amplify the wild-type locus or the modified locus that has been edited by the RGEN.

Standard techniques for transformation of filamentous fungi and culturing the fungi (which are well known to one skilled in the art) are used to transform a fungal host cell of the disclosure. Thus, the introduction of a DNA construct or vector into a fungal host cell includes techniques such as transformation, electroporation, nuclear microinjection, transduction, transfection (e.g., lipofection mediated and DEAE-Dextrin mediated transfection), incubation with calcium phosphate DNA precipitate, high velocity bombardment with DNA-coated microprojectiles, gene gun or biolistic transformation, protoplast fusion and the like. General transformation techniques are known in the art (see, e.g., Ausubel et al., 1987. Sambrook et al., 2001 and 2012, and Campbell et al., 1989). The expression of heterologous proteins in Trichoderma is described, for example, in U.S. Pat. Nos. 6,022,725; 6,268,328; Harkki et al., 1991 and Harkki et al., 1989. Reference is also made to Cao et al. (2000), for transformation of Aspergillus strains.

In certain other embodiments, the recombinant nucleic acid (or polynucleotide expression cassette thereof or expression vector thereof) further comprises one or more selectable markers. Selectable markers for use in filamentous fungi include, but are not limited to, alsl, amdS, hygR, pyr2, pyr4, pyrG, sucA trpC, argB, a bleomycin resistance marker, a blasticidin resistance marker, a pyrithiamine resistance marker, a neomycin resistance marker, an adenine pathway gene, a thymidine kinase marker and the like. In a particular embodiment, the selectable marker is pyr2, which compositions and methods of use are generally set forth in PCT Publication No. WO2011/153449.

Generally, transformation of Trichoderma sp. cells uses protoplasts or cells that have been subjected to a permeability treatment, typically at a density of 10⁵ to 10⁷/mL, particularly 2×10⁶/mL. A volume of 100 μL of these protoplasts or cells in an appropriate solution (e.g., 1.2 M sorbitol and 50 mM CaCl₂) is mixed with the desired DNA. Generally, a high concentration of polyethylene glycol (PEG) is added to the uptake solution. Additives, such as dimethyl sulfoxide, heparin, spermidine, potassium chloride and the like, may also be added to the uptake solution to facilitate transformation. Similar procedures are available for other fungal host cells. See, e.g., U.S. Pat. Nos. 6,022,725 and 6,268,328, both of which are incorporated by reference.

In certain embodiments, the instant disclosure is directed to the expression/production of one or more proteins of interest which are endogenous to the filamentous fungal host cell. In other embodiments, the disclosure is directed to expressing/producing one or more proteins of interest which are heterologous to the filamentous fungal host cell.

In particular embodiments, a heterologous gene (or ORF) encoding a protein (e.g., a regulatory protein, a protein of interest) is introduced into a filamentous fungal (host) cell. In certain embodiments, the heterologous gene is cloned into an intermediate vector, before being transformed into a filamentous fungal (host) cells for expression. These intermediate vectors can be prokaryotic vectors, such as, e.g., plasmids, or shuttle vectors. The expression vector/construct typically contains a transcription unit or expression cassette that contains all the additional elements required for the expression of the heterologous sequence. For example, a typical expression cassette contains a 5′ promoter operably linked to the heterologous nucleic acid sequence encoding the POI and may further comprise sequence signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette may also contain a transcription termination region downstream of the structural gene to provide for efficient termination. The termination region may be obtained from the same gene as the promoter sequence, or may be obtained from different genes. Although any fungal terminator is likely to be functional in the present invention, preferred terminators include: the terminator from Trichoderma cbhI gene, the terminator from Aspergillus nidulans trpC gene (Yelton et al., 1984; Mullaney et al., 1985), the Aspergillus awamori or Aspergillus niger glucoamylase genes (Nunberg et al., 1984; Boel et al., 1984) and/or the Mucor miehei carboxyl protease gene (EPO Publication No. 0215594).

The particular expression vector used to transport the genetic information into the cell is not particularly critical. Any of the conventional vectors used for expression in eukaryotic or prokaryotic cells may be used. Standard bacterial expression vectors include bacteriophages λ and M13, as well as plasmids such as pBR322 based plasmids, pSKF, pET23D, and fusion expression systems such as MBP, GST, and LacZ, as well as yeast 2μ plasmids and centromeric yeast plasmids. Epitope tags can also be added to recombinant proteins to provide convenient methods of isolation, e.g., c-myc.

The elements that can be included in expression vectors may also be a replicon, a gene encoding antibiotic resistance to permit selection of bacteria that harbor recombinant plasmids, or unique restriction sites in nonessential regions of the plasmid to allow insertion of heterologous sequences. The particular antibiotic resistance gene chosen is not dispositive either, as any of the many resistance genes known in the art may be suitable. The prokaryotic sequences are preferably chosen such that they do not interfere with the replication or integration of the DNA in the fungal host.

The methods of transformation of the present disclosure may result in the stable integration of all or part of the transformation vector into the genome of the filamentous fungus. However, transformation resulting in the maintenance of a self-replicating extra-chromosomal transformation vector is also contemplated. Any of the known procedures for introducing foreign (heterologous) nucleotide sequences into host cells may be used. These include the use of calcium phosphate transfection, polybrene, protoplast fusion, electroporation, biolistics, liposomes, microinjection, and any of the other known methods for introducing cloned genomic DNA, cDNA, synthetic DNA or other foreign genetic material into a host cell (see, e.g., Sambrook et al., supra). Also of use is the Agrobacterium-mediated transfection method such as the one described in U.S. Pat. No. 6,255,115.

After the expression vector(s) is/are introduced into the cells, the transfected cells are cultured under conditions favoring expression of genes under control of cellulase gene promoter sequences. Large batches of transformed cells can be cultured as described herein. Finally, product is recovered from the culture using standard techniques.

### V. Proteins of Interest

As stated above, certain embodiments of the disclosure are related to genetically modified (recombinant) fungal cells comprising genetic modifications which expresses or overexpress a gene (or ORF) encoding a protein of interest (POI). More particularly, certain embodiments are related to compositions and methods for the expression/production of such proteins of interest in the modified (mutant) fungal cells of the disclosure. Thus, in certain embodiments recombinant fungal cells of the disclosure produced increased amounts of proteins of interest, including, but not limited to, enzymes, antibodies, receptor proteins, animal feed proteins and/or human food proteins.

In certain aspects, recombinant cells of the disclosure produce increased amounts of an enzyme elected from the group consisting of amylases, cellulases, hemicellulases, xylanases, peroxidases, proteases, lipases, phospholipases, esterases, cutinases, polyesterases, phytase, pectinases, keratinases, reductases, oxidases, phenol oxidases, lipoxygenases, ligninases, pullulanases, tannases, pentosanases, mannanases, α-glucanases, β-glucanases, hyaluronidases, chondroitinases, laccases, amylases, glucoamylases, acetyl esterases, aminopeptidase, arabinases, arabinosidases, arabinofuranosidases, carboxypeptidases, catalases, nucleases, deoxyribonucleases, ribonucleases, epimerases, α-galactosidases, β-galactosidases, glucan lysases, endo-β-glucanases, glucose oxidases, glucuronidases, invertases, and isomerases.

In other embodiments, recombinant cells of the disclosure produce increased amounts of an amylase selected from the group consisting of α-amylases, β-amylases, maltogenic α-amylases, α-glucosidases glucoamylase, maltotetraosidases and maltohexaosidases.

As described herein, a protein of interest (POI) may be an endogenous POI or a heterologous POI. In certain embodiments, a POI includes, but is not limited to, a hemicellulase, a peroxidase, a protease, a cellulase, a xylanase, a lipase, a phospholipase, an esterase, a cutinase, a pectinase, a keratinase, a reductase, an oxidase, a phenol oxidase, a lipoxygenase, a ligninase, a pullulanase, a tannase, a pentosanase, a mannanase, a β-glucanase, a hyaluronidase, a chondroitinase, a laccase, a amylase, a glucoamylase, an acetyl esterase, an aminopeptidase, amylases, an arabinases, an arabinosidase, an arabinofuranosidase, a carboxypeptidase, a catalase, a deoxyribonuclease, an epimerase, an α-galactosidase, a β-galactosidase, an α-glucanases, a glucan lysase, an endo-β-glucanase, a glucose oxidase, a glucuronidase, an invertase, an isomerase, and the like. In certain embodiments, a POI is selected from an Enzyme Commission (EC) Number selected from the group consisting of EC 1, EC 2, EC 3, EC 4, EC 5 or EC 6.

For example, in certain embodiments a POI is an oxidoreductase enzyme, including, but not limited to, an EC1 (oxidoreductase) enzyme selected from EC 1.10.3.2 (e.g., a laccase), EC 1.10.3.3 (e.g., L-ascorbate oxidase), EC 1.1.1.1 (e.g., alcohol dehydrogenase), EC 1.11.1.10 (e.g., chloride peroxidase), EC 1.11.1.17 (e.g., peroxidase), EC 1.1.1.27 (e.g., L-lactate dehydrogenase), EC 1.1.1.47 (e.g., glucose 1-dehydrogenase), EC 1.1.3.X (e.g., glucose oxidase), EC 1.1.3.10 (e.g., pyranose oxidase), EC 1.13.11.X (e.g., dioxygenase), EC 1.13.11.12 (e.g., lineolate 13S-lipozygenase), EC 1.1.3.13 (e.g., alcohol oxidase), EC 1.14.14.1 (e.g., monooxygenase), EC 1.14.18.1 (e.g., monophenol monooxigenase), EC 1.15.1.1 (e.g., superoxide dismutase), EC 1.1.5.9 (formerly EC 1.1.99.10, e.g., glucose dehydrogenase), EC 1.1.99.18 (e.g., cellobiose dehydrogenase), EC 1.1.99.29 (e.g., pyranose dehydrogenase), EC 1.2.1.X (e.g., fatty acid reductase), EC 1.2.1.10 (e.g., acetaldehyde dehydrogenase), EC 1.5.3.X (e.g., fructosyl amine reductase), EC 1.8, 1.X (e.g., disulfide reductase) and EC 1.8.3.2 (e.g., thiol oxidase).

In certain embodiments a POI is a transferase enzyme, including, but not limited to, an EC 2 (transferase) enzyme selected from EC 2.3.2.13 (e.g., transglutaminase), EC 2.4.1.X (e.g., hexosyltransferase), EC 2.4.1.40 (e.g., altemasucrase), EC 2.4.1.18 (e.g., 1,4 alpha-glucan branching enzyme), EC 2.4.1.19 (e.g., cyclomaltodextrin glucanotransferase), EC 2.4.1.2 (e.g., dextrin dextranase), EC 2.4.1.20 (e.g., cellobiose phosphorylase), EC 2.4.1.25 (e.g., 4-alpha-glucanotransferase), EC 2.4.1.333 (e.g., 1,2-beta-oligoglucan phosphor transferase), EC 2.4.1.4 (e.g., amylosucrase), EC 2.4.1.5 (e.g., dextransucrase), EC 2.4.1.69 (e.g., galactoside 2-alpha-L-fucosyl transferase), EC 2.4.1.9 (e.g., inulosucrase), EC 2.7.1.17 (e.g., xylulokinase), EC 2.7.7.89 (formerly EC 3.1.4.15, e.g., [glutamine synthetase]-adenylyl-L-tyrosine phosphorylase), EC 2.7.9.4 (e.g., alpha glucan kinase) and EC 2.7.9.5 (e.g., phosphoglucan kinase).

In other embodiments a POI is a hydrolase enzyme, including, but not limited to, an EC 3 (hydrolase) enzyme selected from EC 3.1.X.X (e.g., an esterase), EC 3.1.1.1 (e.g., pectinase), EC 3.1.1.14 (e.g., chlorophyllase), EC 3.1.1.20 (e.g., tannase), EC 3.1.1.23 (e.g., glycerol-ester acylhydrolase), EC 3.1.1.26 (e.g., galactolipase), EC 3.1.1.32 (e.g., phospholipase A1), EC 3.1.1.4 (e.g., phospholipase A2), EC 3.1.1.6 (e.g., acetylesterase), EC 3.1.1.72 (e.g., acetylxylan esterase), EC 3.1.1.73 (e.g., feruloyl esterase), EC 3.1.1.74 (e.g., cutinase), EC 3.1.1.86 (e.g., rhamnogalacturonan acetylesterase), EC 3.1.1.87 (e.g., fumosin Bl esterase), EC 3.1.26.5 (e.g., ribonuclease P), EC 3.1.3.X (e.g., phosphoric monoester hydrolase), EC 3.1.30.1 (e.g., Aspergillus nuclease Sl), EC 3.1.30.2 (e.g., Serratia marcescens nuclease), EC 3.1.3.1 (e.g., alkaline phosphatase), EC 3.1.3.2 (e.g., acid phosphatase), EC 3.1.3.8 (e.g., 3-phytase), EC 3.1.4.1 (e.g., phosphodiesterase I), EC 3.1.4.11 (e.g., phosphoinositide phospholipase C), EC 3.1.4.3 (e.g., phospholipase C), EC 3.1.4.4 (e.g., phospholipase D), EC 3.1.6.1 (e.g., arylsulfatase), EC 3.1.8.2 (e.g., diisopropyl-fluorophosphatase), EC 3.2.1.10 (e.g., oligo-1,6-glucosidase), EC 3.2.1.101 (e.g., mannanendo-1,6-alpha-mannosidase), EC 3.2.1.11 (e.g., alpha-1,6-glucan-6-glucanohydrolase), EC 3.2.1.131 (e.g., xylan alpha-1,2-glucuronidase), EC 3.2.1.132 (e.g., chitosan N-acetylglucosaminohydrolase), EC 3.2.1.139 (e.g., alpha-glucuronidase), EC 3.2.1.14 (e.g., chitinase), EC 3.2.1.151 (e.g., xyloglucan-specific endo-beta-1,4-glucanase), EC 3.2.1.155 (e.g., xyloglucan-specific exo-beta-1,4-glucanase), EC 3.2.1.164 (e.g., galactan endo-1,6-beta-galactosidase), EC 3.2.1.17 (e.g., lysozyme), EC 3.2.1.171 (e.g., rhamnogalacturonan hydrolase), EC 3.2.1.174 (e.g., rhamnogalacturonan rhamnohydrolase), EC 3.2.1.2 (e.g., beta-amylase), EC 3.2.1.20 (e.g., alpha-glucosidase), EC 3.2.1.22 (e.g., alpha-galactosidase), EC 3.2.1.25 (e.g., beta-mannosidase), EC 3.2.1.26 (e.g., beta-fructofuranosidase), EC 3.2.1.37 (e.g., xylan 1,4-beta-xylosidase), EC 3.2.1.39 (e.g., glucan endo-1,3-beta-D-glucosidase), EC 3.2.1.40 (e.g., alpha-L-rhamnosidase), EC 3.2.1.51 (e.g., alpha-L-fucosidase), EC 3.2.1.52 (e.g., beta-N-Acetylhexosaminidase), EC 3.2.1.55 (e.g., alpha-N-arabinofuranosidase), EC 3.2.1.58 (e.g., glucan 1,3-beta-glucosidase), EC 3.2.1.59 (e.g., glucan endo-1,3-alpha-glucosidase), EC 3.2.1.67 (e.g., galacturan 1,4-alpha-galacturonidase), EC 3.2.1.68 (e.g., isoamylase), EC 3.2.1.7 (e.g., 1-beta-D-fructan fructanohydrolase), EC 3.2.1.74 (e.g., glucan 1,4-glucosidase), EC 3.2.1.75 (e.g., glucan endo-1,6-beta-glucosidase), EC 3.2.1.77 (e.g., mannan 1,2-(1,3)-alpha-mannosidase), EC 3.2.1.80 (e.g., fructan beta-fructosidase), EC 3.2.1.82 (e.g., exo-poly-alpha-galacturonosidase), EC 3.2.1.83 (e.g., kappa-carrageenase), EC 3.2.1.89 (e.g., arabinogalactan endo-1,4-beta-galactosidase), EC 3.2.1.91 (e.g., cellulose 1,4-beta-cellobiosidase), EC 3.2.1.96 (e.g., mannosyl-glycoprotein endo-beta-N-acetylglucosaminidase), EC 3.2.1.99 (e.g., arabinan endo-1,5-alpha-L-arabinanase), EC 3.4.X.X (e.g., peptidase), EC 3.4.1 1.X (e.g., aminopeptidase), EC 3.4.11.1 (e.g., leucyl aminopeptidase), EC 3.4.11.18 (e.g., methionyl aminopeptidase), EC 3.4.13.9 (e.g., Xaa-Pro dipeptidase), EC 3.4.14.5 (e.g., dipeptidyl-peptidase IV), EC 3.4.16. X (e.g., serine-type carboxypeptidase), EC 3.4.16.5 (e.g., carboxypeptidase C), EC 3.4.19.3 (e.g., pyroglutamyl-peptidase I), EC 3.4.21. X (e.g., serine endopeptidase), EC 3.4.21.1 (e.g., chymotrypsin), EC 3.4.21.19 (e.g., glutamyl endopeptidase), EC 3.4.21.26 (e.g., prolyl oligopeptidase), EC 3.4.21.4 (e.g., trypsin), EC 3.4.21.5 (e.g., thrombin), EC 3.4.21.63 (e.g., oryzin), EC 3.4.21.65 (e.g., thermomycolin), EC 3.4.21.80 (e.g., streptogrisin A), EC 3.4.22. X (e.g., cysteine endopeptidase), EC 3.4.22.14 (e.g., actinidain), EC 3.4.22.2 (e.g., papain), EC 3.4.22.3 (e.g., ficain), EC 3.4.22.32 (e.g., stem bromelain), EC 3.4.22.33 (e.g., fruit bromelain), EC 3.4.22.6 (e.g., chymopapain), EC 3.4.23.1 (e.g., pepsin A), EC 3.4.23.2 (e.g., pepsin B), EC 3.4.23.22 (e.g., endothiapepsin), EC 3.4.23.23 (e.g., mucorpepsin), EC 3.4.23.3 (e.g., gastricsin), EC 3.4.24.X (e.g., metalloendopeptidase), EC 3.4.24.39 (e.g., deuterolysin), EC 3.4.24.40 (e.g., serralysin), EC 3.5.1.1 (e.g., asparaginase), EC 3.5.1.11 (e.g., penicillin amidase), EC 3.5.1.14 (e.g., N-acyl-aliphatic-L-amino acid amidohydrolase), EC 3.5.1.2 (e.g., L-glutamine amidohydrolase), EC 3.5.1.28 (e.g., N-acetylmuramoyl-L-alanine amidase), EC 3.5.1.4 (e.g., amidase), EC 3.5.1.44 (e.g., protein-L-glutamine amidohydrolase), EC 3.5.1.5 (e.g., urease), EC 3.5.1.52 (e.g., peptide-N(4)-(N-acetyl-beta-glucosaminyl)asparagine amidase), EC 3.5.1.81 (e.g., N-Acyl-D-amino-acid deacylase), EC 3.5.4.6 (e.g., AMP deaminase) and EC 3.5.5.1 (e.g., nitrilase).

In other embodiments a POI is a lyase enzyme, including, but not limited to, an EC 4 (lyase) enzyme selected from EC 4.1.2.10 (e.g., mandelonitrile lyase), EC 4.1.3.3 (e.g., N-acetylneuraminate lyase), EC 4.2.1.1 (e.g., carbonate dehydratase), EC 4.2.2.—(e.g., rhamnogalacturonan lyase), EC 4.2.2.10 (e.g., pectin lyase), EC 4.2.2.22 (e.g., pectate trisaccharide-lyase), EC 4.2.2.23 (e.g., rhamnogalacturonan endolyase) and EC 4.2.2.3 (e.g., mannuronate-specific alginate lyase).

In certain other embodiments a POI is an isomerase enzyme, including, but not limited to, an EC 5 (isomerase) enzyme selected from EC 5.1.3.3 (e.g., aldose 1-epimerase), EC 5.1.3.30 (e.g., D-psicose 3-epimerase), EC 5.4.99.11 (e.g., isomaltulose synthase) and EC 5.4.99.15 (e.g., (1 4)-a-D-glucan 1-a-D-glucosylmutase).

In yet other embodiments, a POI is a ligase enzyme, including, but not limited to, an EC 6 (ligase) enzyme selected from EC 6.2.1.12 (e.g., 4-coumarate: coenzyme A ligase) and EC 6.3.2.28 (e.g., L-amino-acid alpha-ligase).

Optimal conditions for the production of the proteins will vary with the choice of the host cell, and with the choice of the protein(s) to be expressed. Such conditions may be readily ascertained by one skilled in the art through routine experimentation and/or optimization.

The protein of interest can be purified or isolated after expression. The protein of interest may be isolated or purified in a variety of ways known to those skilled in the art depending on what other components are present in the sample. Standard purification methods include, but are not limited to, electrophoretic, molecular, immunological and chromatographic techniques, including ion exchange, hydrophobic, affinity, and reverse-phase HPLC chromatography, and chromatofocusing. For example, the protein of interest may be purified using a standard anti-protein of interest antibody column. Ultrafiltration and diafiltration techniques, in conjunction with protein concentration, are also useful. The degree of purification necessary will vary depending on the intended use of the protein of interest. In certain instances, no purification of the protein will be necessary.

In certain other embodiments, to confirm that a genetically modified fungal cell of the disclosure produces an increased level of a protein of interest, various methods of screening may be performed. The expression vector may encode a polypeptide fusion to the target protein which serves as a detectable label or the target protein itself may serve as the selectable or screenable marker. The labeled protein may be detected via western blotting, dot blotting (methods available at the Cold Spring Harbor Protocols website), ELISA, or, if the label is GFP, whole cell fluorescence and/or FACS. For example, a 6-histidine tag would be included as a fusion to the target protein, and this tag would be detected by western blotting. If the target protein expresses at sufficiently high levels, SDS-PAGE combined with Coomassie/silver staining, may be performed to detect increases in variant host cell expression over parental (control) cell, in which case no label is necessary. In addition, other methods may be used to confirm the improved level of a protein of interest, such as, the detection of the increase of protein activity or amount per cell, protein activity or amount per milliliter of medium, allowing cultures or fermentations to continue efficiently for longer periods of time, or through a combination of these methods.

The detection of specific productivity is another method to evaluate the protein production. Specific productivity (Qp) can be determined by the following equation:

$$Qp = gP / gDCW * hr$$

wherein “gP” is grams of protein produced in the tank, “gDCW” is grams of dry cell weight (DCW) in the tank, “hr” is fermentation time in hours from the time of inoculation, which include the time of production as well as growth time.

### VI. Fermentation

Certain embodiments are related to compositions and methods for producing a protein of interest comprising growing, cultivating or fermenting a modified (mutant) filamentous fungal cell of the disclosure. In general, fermentation methods well known in the art are used to ferment the fungal cells. In some embodiments, the fungal cells are grown under batch, fed-batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation occurs without the addition of any components to the system. Typically, a batch fermentation qualifies as a “batch” with respect to the addition of the nutrients, while factors such as pH and oxygen concentration are controlled. The broth and culture compositions of the batch system change constantly up to the time the fermentation is stopped. Within batch cultures, cells progress through a static lag phase to a high growth log phase and finally to a stationary phase, where growth rate is diminished or halted. If untreated, cells proceed to apoptosis and eventually die. In general, in the batch phase, the bulk of the production of product occurs during the log phase

A suitable variation on the standard batch system is the “fed-batch fermentation” system. In this variation of a typical batch system, after the log phase is finished, the substrate is added in increments as the fermentation progresses. Fed-batch systems are often used to avoid catabolite repression. Continuous feeding of the substrate allows the process to keep its concentration below critical level that could lead to inhibition of cellular metabolism and protein production. Batch and fed-batch fermentations are common and well known in the art.

Continuous fermentation is a system where a defined fermentation medium is added continuously to a bioreactor, and an equal amount of conditioned medium is removed simultaneously for processing. Continuous fermentation generally maintains the cultures at a constant (high) density, where cells are primarily kept in log phase growth. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

Certain embodiments of the instant disclosure are related to fermentation procedures for culturing fungi. Fermentation procedures for production of cellulase enzymes are known in the art. For example, cellulase enzymes can be produced either by solid or submerged culture, including batch, fed-batch and continuous-flow processes. Culturing is generally accomplished in a growth medium comprising an aqueous mineral salts medium, organic growth factors, a carbon and energy source material, molecular oxygen, and, of course, a starting inoculum of the filamentous fungal host to be employed.

In addition to the carbon and energy source, oxygen, assimilable nitrogen, and an inoculum of the microorganism, it is necessary to supply suitable amounts in proper proportions of mineral nutrients to assure proper microorganism growth, maximize the assimilation of the carbon and energy source by the cells in the microbial conversion process, and achieve maximum cellular yields with maximum cell density in the fermentation media.

The composition of the aqueous mineral medium can vary over a wide range, depending in part on the microorganism and substrate employed, as is known in the art. The mineral media should include, in addition to nitrogen, suitable amounts of phosphorus, magnesium, calcium, potassium, sulfur, and sodium, in suitable soluble assimilable ionic and combined forms, and also present preferably should be certain trace elements such as copper, manganese, molybdenum, zinc, iron, boron, and iodine, and others, again in suitable soluble assimilable form, all as known in the art.

The fermentation process can be an aerobic process in which the molecular oxygen needed is supplied by a molecular oxygen-containing gas such as air, oxygen-enriched air, or even substantially pure molecular oxygen, provided to maintain the contents of the fermentation vessel with a suitable oxygen partial pressure effective in assisting the microorganism species to grow in a thriving fashion.

The fermentation temperature can vary somewhat, but for filamentous fungi such as Trichoderma reesei, the temperature generally will be within the range of about 20° C. to 40° C., generally preferably in the range of about 25° C. to 34° C.

The microorganisms also require a source of assimilable nitrogen. The source of assimilable nitrogen can be any nitrogen-containing compound or compounds capable of releasing nitrogen in a form suitable for metabolic utilization by the microorganism. While a variety of organic nitrogen source compounds, such as protein hydrolysates, can be employed, usually cheap nitrogen-containing compounds such as ammonia, ammonium hydroxide, urea, and various ammonium salts such as ammonium phosphate, ammonium sulfate, ammonium pyrophosphate, ammonium chloride, or various other ammonium compounds can be utilized. Ammonia gas itself is convenient for large scale operations, and can be employed by bubbling through the aqueous ferment (fermentation medium) in suitable amounts. At the same time, such ammonia can also be employed to assist in pH control.

The pH range in the aqueous microbial ferment should be in the exemplary range of about 2.0 to 10.0. With filamentous fungi, the pH normally is within the range of about 2.5 to 8.0; with Trichoderma reesei, the pH normally is within the range of about 3.0 to 7.0. Preferences for pH range of microorganisms are dependent on the media employed to some extent, as well as the particular microorganism, and thus can be somewhat adjusted as can be readily determined by those skilled in the art.

Preferably, the fermentation is conducted in such a manner that the carbon-containing substrate can be controlled as a limiting factor, thereby providing good conversion of the carbon-containing substrate to products and avoiding contamination of the cells with a substantial amount of unconverted substrate. The latter is not a problem with water-soluble substrates, since any remaining traces are readily washed off. It may be a problem, however, in the case of non-water-soluble substrates, and require added product-treatment steps such as suitable washing steps.

As described above, the time to reach this level is not critical and may vary with the particular microorganism and fermentation process being conducted. However, it is well known in the art how to determine the carbon source concentration in the fermentation medium and whether or not the desired level of carbon source has been achieved.

The fermentation can be conducted as a batch or continuous operation, fed batch operation is much to be preferred for ease of control, production of uniform quantities of products, and most economical uses of all equipment.

If desired, part or all of the carbon and energy source material and/or part of the assimilable nitrogen source such as ammonia can be added to the aqueous mineral medium prior to feeding the aqueous mineral medium to the fermenter.

Each of the streams introduced into the reactor preferably is controlled at a predetermined rate, or in response to a need determinable by monitoring such as concentration of the carbon and energy substrate, pH, dissolved oxygen, oxygen or carbon dioxide in the off-gases from the fermenter, cell density measurable by dry cell weights, light transmittancy, or the like. The feed rates of the various materials can be varied so as to obtain maximal production rates and/or maximum yields.

In either a batch, or the preferred fed batch operation, all equipment, reactor, or fermentation means, vessel or container, piping, attendant circulating or cooling devices, and the like, are initially sterilized, usually by employing steam such as at about 121° C. for at least about 15 minutes. The sterilized reactor then is inoculated with a culture of the selected microorganism in the presence of all the required nutrients, including oxygen, and the carbon-containing substrate. The type of fermenter employed is not critical.

The collection and purification of (e.g., cellulase) enzymes from the fermentation broth can also be done by procedures known to one skilled in the art. The fermentation broth will generally contain cellular debris, including cells, various suspended solids and other biomass contaminants, as well as the desired cellulase enzyme product, which are preferably removed from the fermentation broth by means known in the art.

Suitable processes for such removal include conventional solid-liquid separation techniques such as, e.g., centrifugation, filtration, dialysis, microfiltration, rotary vacuum filtration, or other known processes, to produce a cell-free filtrate. It may be preferable to further concentrate the fermentation broth or the cell-free filtrate prior to crystallization using techniques such as ultrafiltration, evaporation or precipitation.

Precipitating the proteinaceous components of the supernatant or filtrate may be accomplished by means of a salt, e.g., ammonium sulfate, followed by purification by a variety of chromatographic procedures, e.g., ion exchange chromatography, affinity chromatography or similar art recognized procedures.

### VII. EXEMPLARY EMBODIMENTS

Non-limiting embodiments of compositions and methods disclosed herein are as follows:

1. A recombinant fungal cell overexpressing a gene comprising at least 80% identity to a Trichoderma reesei gene of SEQ ID NO: 1, 4, 7, 10, 12, 15, 18, 21, 23, 25, 28, 30, 33, 36, 39, 42, 45, 48, 51, 54, 56, 59, 62, 65, 68, 71, 74, 77, 79, 82, 84, 87, 90, 94, 97, 99, 101, 105, 108, 110, 113, 116, 119, 122, 125, 128,131, 134, 137, 140, 144, 147, 149 or 152.

2. A recombinant fungal cell overexpressing a gene comprising at least 80% identity a gene sequence shown in TABLE 6.

3. A recombinant fungal cell overexpressing a protein comprising at least 80% identity to a Trichoderma reesei protein of SEQ ID NO: 2, 5, 8, 11, 13, 16, 19, 22, 24, 26, 29, 31, 34, 37, 40, 43, 46, 49, 52, 55, 57, 60, 63, 66, 69, 72, 75, 78, 80, 83, 85, 88, 91, 95, 98, 100, 102, 106, 109, 111, 114, 117, 120, 123, 126, 129,132, 135, 138, 141, 145, 148, 150 or 153.

4. A recombinant fungal cell overexpressing a protein comprising at least 80% identity to a protein shown in TABLE 6.

5. A recombinant fungal cell overexpressing a combination of at least two genes comprising at least 80% identity to a Trichoderma reesei gene sequence combination shown in TABLE 4 or TABLE 5.

6. A recombinant fungal cell overexpressing a combination of at least two proteins comprising at least 80% identity to a Trichoderma reesei protein combination shown in TABLE 4 or TABLE 5.

7. A polynucleotide (e.g., an expression cassette) comprising an upstream (5′) promoter sequence operably linked to a downstream (3′) nucleic acid sequence encoding a protein comprising at least 80% sequence identity to a protein of SEQ ID NO: 2, 5, 8, 11, 13, 16, 19, 22, 24, 26, 29, 31, 34, 37, 40, 43, 46, 49, 52, 55, 57, 60, 63, 66, 69, 72, 75, 78, 80, 83, 85, 88, 91, 95, 98, 100, 102, 106, 109, 111, 114, 117, 120, 123, 126, 129,132, 135, 138, 141, 145, 148, 150 or 153.

8. A polynucleotide comprising an upstream (5′) promoter sequence operably linked to a downstream (3′) nucleic acid sequence encoding a protein comprising at least 80% sequence identity to a protein ortholog shown in TABLE 6.

9. The polynucleotide of embodiment 7 or 8, wherein the upstream promoter is a constitutive promoter or an inducible promoter.

10. The polynucleotide of embodiment 7 or 8, further comprising a terminator sequence downstream (3′) and operably linked to nucleic acid sequence encoding the protein.

11. A recombinant fungal cell comprising a polynucleotide of embodiment 7 or 8.

12. A recombinant fungal cell comprising (a) polynucleotide of embodiment 7 or 8, and (b) a polynucleotide encoding a protein of interest (POI).

13. The recombinant cell of embodiment 12, wherein the polynucleotide encoding the POI comprises an upstream (5′) promoter sequence operably linked to downstream (3′) nucleic acid sequence encoding the POI.

14. A recombinant fungal cell producing a protein of interest (POI) and overexpressing a protein comprising at least 80% identity to a protein of SEQ ID NO: 2, 5, 8, 11, 13, 16, 19, 22, 24, 26, 29, 31, 34, 37, 40, 43, 46, 49, 52, 55, 57, 60, 63, 66, 69, 72, 75, 78, 80, 83, 85, 88, 91, 95, 98, 100, 102, 106, 109, 111, 114, 117, 120, 123, 126, 129, 132, 135, 138, 141, 145, 148, 150 or 153.

15. A recombinant fungal cell producing a protein of interest (POI) and overexpressing a protein encoded by a gene comprising at least 80% identity to a gene sequence shown in TABLE 6.

16. A recombinant fungal cell producing a protein of interest (POI) and overexpressing a combination at least two genes comprising at least 80% identity to a Trichoderma reesei gene sequence of combination shown in TABLE 4 or TABLE 5.

17. A recombinant fungal cell producing a protein of interest (POI) and overexpressing a combination of at least two proteins comprising at least 80% identity to a Trichoderma reesei protein of combination shown in TABLE 4 or TABLE 5.

18. The recombinant cell of any one of embodiments 11-17, wherein the protein of interest (POI) is selected from the group consisting of an enzyme, an antibody, a receptor protein, an animal feed protein and a human food protein.

19. The recombinant cell of embodiment 18, wherein the enzyme is selected from the group consisting of amylases, cellulases, hemicellulases, xylanases, peroxidases, proteases, lipases, phospholipases, esterases, cutinases, polyesterases, phytase, pectinases, keratinases, reductases, oxidases, phenol oxidases, lipoxygenases, ligninases, pullulanases, tannases, pentosanases, mannanases, α-glucanases, β-glucanases, hyaluronidases, chondroitinases, laccases, amylases, glucoamylases, acetyl esterases, aminopeptidase, arabinases, arabinosidases, arabinofuranosidases, carboxypeptidases, catalases, nucleases, deoxyribonucleases, ribonucleases, epimerases, α-galactosidases, β-galactosidases, glucan lysases, endo-β-glucanases, glucose oxidases, glucuronidases, invertases, and isomerases.

20. The recombinant cell of embodiment 18, wherein the enzyme is an amylase selected from the group consisting of α-amylases, β-amylases, maltogenic α-amylases, α-glucosidases, glucoamylases, maltotetraosidases and maltohexaosidases.

21. A recombinant fungal cell overexpressing a protein comprising at least 80% identity to SEQ ID NO: 68, wherein the recombinant cell comprises an improved sporulation phenotype relative to a control fungal cell which does not overexpress any regulatory proteins.

22. A method for producing an increased amount of a heterologous protein of interest (POI) in a fungal cell comprising: (a) constructing (or obtaining) a recombinant fungal cell comprising a polynucleotide encoding a protein of interest (POI), wherein the polynucleotide comprises an upstream (5′) promoter sequence operably linked to a downstream (3′) nucleic acid sequence encoding the POI, (b) introducing into the cell a polynucleotide comprising an upstream (5′) promoter sequence operably linked to a downstream (3′) nucleic acid sequence encoding a protein comprising at least 80% sequence identity to a protein of SEQ ID NO: 2, 5, 13, 16, 19, 24, 26, 31, 34, 40, 49, 55, 57, 60, 63, 66, 69, 75, 85, 88, 95, 98, 100, 102, 106, 111, 114, 117, 123, 129,132, 135, 138, 141, 145, 148, 150 or 153, or a protein comprising at least 80% identity to a protein ortholog shown in TABLE 6, and (c) fermenting the recombinant cell, wherein the recombinant cell of step (b) produces an increased amount of the POI relative to the recombinant cell of step (a), when the cells are fermented under the same conditions for the production of the POI.

23. A method for producing an increased amount of a heterologous protein of interest (POI) in a fungal cell comprising: (a) constructing (or obtaining) a recombinant fungal cell comprising a polynucleotide encoding a POI, wherein the polynucleotide comprises an upstream (5′) promoter sequence operably linked to downstream (3′) nucleic acid sequence encoding the POI, (b) introducing into the cell a polynucleotide comprising an upstream (5′) promoter sequence operably linked to a downstream (3′) nucleic acid sequence encoding a protein comprising at least 80% sequence identity to a protein of SEQ ID NO: SEQ ID NO: 5, 8, 11, 16, 19, 22, 24, 29, 31, 37, 43, 46, 52, 55, 57, 66, 69, 72, 75, 78, 80, 83, 85, 91, 95, 100, 102, 106, 109, 114, 117, 120, 123, 126 or 129, or a protein comprising at least 80% identity to a protein ortholog shown in TABLE 6, and (c) fermenting the recombinant cell at an elevated fermentation temperature, wherein the recombinant cell of step (b) produces an increased amount of the POI relative to the recombinant cell of step (a), when the cells are fermented under the same conditions for the production of the POI.

24. A method for producing an increased amount of a heterologous protein of interest (POI) in a fungal cell comprising: (a) constructing (or obtaining) a recombinant fungal cell overexpressing a combination of at least two proteins comprising at least 80% identity to a protein sequence combination shown in TABLE 4 or TABLE 5, (b) introducing into the cell a polynucleotide encoding a POI, wherein the polynucleotide comprises an upstream (5′) promoter sequence operably linked to downstream (3′) nucleic acid sequence encoding the POI, and (c) fermenting the cell, wherein the recombinant cell produces an increased amount of the POI relative to a control cell when fermented under the same conditions for the production of the POI, wherein control cell comprises the expression cassette encoding the POI, but does not overexpress the combination of at least two genes.

25. The method of any one of embodiments 22-24, wherein the polynucleotide encoding the POI is integrated into the genome of the fungal cell and/or the polynucleotide encoding the regulatory protein is integrated into the genome of the fungal cell.

26. The method of any one of embodiments 22-24, wherein the protein of interest (POI) is selected from the group consisting of an enzyme, an antibody, a receptor protein, an animal feed protein and a human food protein.

27. The method of embodiment 26, wherein the enzyme is selected from the group consisting of amylases, cellulases, hemicellulases, xylanases, peroxidases, proteases, lipases, phospholipases, esterases, cutinases, polyesterases, phytase, pectinases, keratinases, reductases, oxidases, phenol oxidases, lipoxygenases, ligninases, pullulanases, tannases, pentosanases, mannanases, α-glucanases, β-glucanases, hyaluronidases, chondroitinases, laccases, amylases, glucoamylases, acetyl esterases, aminopeptidase, arabinases, arabinosidases, arabinofuranosidases, carboxypeptidases, catalases, nucleases, deoxyribonucleases, ribonucleases, epimerases, α-galactosidases, β-galactosidases, glucan lysases, endo-β-glucanases, glucose oxidases, glucuronidases, invertases, and isomerases.

28. The method of embodiment 26, wherein the enzyme is an amylase selected from the group consisting of α-amylases, β-amylases, maltogenic α-amylases, α-glucosidases, glucoamylases, maltotetraosidases and maltohexaosidases.

29. A method for enhancing sporulation of a fungal cell comprising (a) constructing or obtaining a recombinant fungal cell overexpressing a protein comprising at least 80% identity to SEQ ID NO: 68, and (b) growing strain under appropriate sporulation conditions (including, but not limited to, conditions such as nutrient medium, temperature and light), wherein the recombinant cell comprises an enhanced sporulation phenotype as compared to a control fungal cell which does not overexpress the protein comprising at least 80% identity to SEQ ID NO: 68.

### EXAMPLES

It should be understood that the following Examples, while indicating embodiments of the disclosure, are given by way of illustration only. From the above discussion and these Examples, one of skill in the art can make various changes and modifications of the disclosure to adapt it to various usages and conditions. Such modifications are also intended to fall within the scope of the claimed invention. Standard recombinant DNA and molecular cloning techniques used herein are well known in the art (Ausubel et al., 1987; Sambrook et al., 1989).

### Example 1

### Construction of a Regulatory Protein Overexpression Library in Filamentous Fungal Cells

### A. Overview

As generally set forth above in the Detailed Description, regulatory proteins are key regulators of special cellular functions such as signaling, chemotaxis, transport, metabolic regulation, gene expression, protein degradation, Many regulatory proteins typically regulate gene expression by binding to specific (target) sites in the promoter regions of the (regulated) genes. The instant example generally describes the design and construction of exemplary T. reesei reporter strains, and modified (recombinant) strains thereof overexpressing a regulatory protein (TABLE 1) or a combination of two regulatory proteins thereof.

B. Trichoderma reesei Reporter Strain

The T. reesei host cells set forth and described in the following examples were derived from T. reesei strain RL-P37 (NRRL Deposit No. 15709), wherein the T. reesei pyr2 gene has been deleted (Δpyr2), as generally described by Sheir-Neiss and Montenecourt (1984).

The T. reesei host cells were further modified to overexpress two (2) reporter proteins, (i) an engineered phytase from Buttiauxella sp. and (ii) an alpha-amylase from Aspergillus terreus under the control of the T. reesei cbhI regulatory sequences. For example, the DNA constructs carrying synthetic genes coding for the above-mentioned reporter proteins were co-transformed together with a selectable marker amdS from Aspergillus nidulans operably linked to one of the constructs. The correct clones were validated by PCR amplification, which confirmed presence of both genes (i.e., synthetic DNA constructs) in the genome. Production and functionality of the heterologous reporter proteins was confirmed by enzymatic activity measurements of fermentation broth.

### C. Construction of Regulatory Proteins Expression Vectors

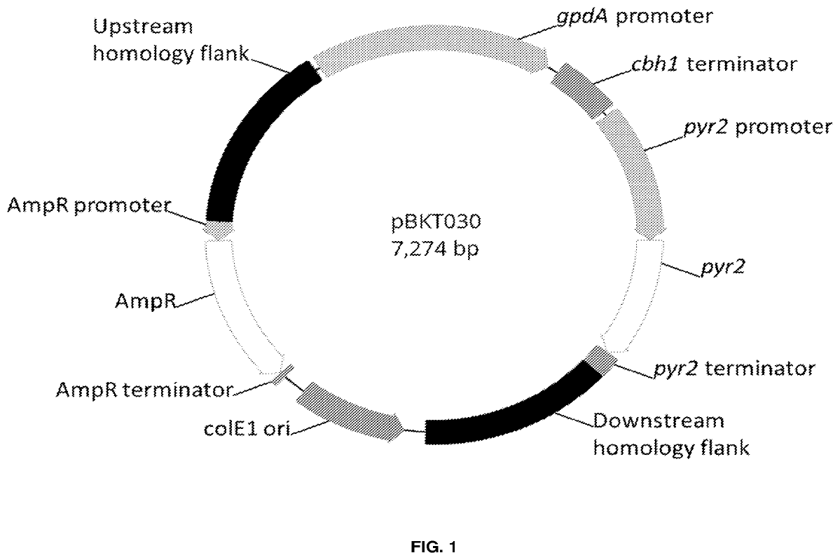

In the instant example, Applicant identified more than 750 regulatory proteins which were selected with use of the bioinformatics tools from the JGI database for Trichoderma reesei QM6a strain. For some of the genes an alternative start and/or stop codon(s) was/were identified. In such a case, different putative variants of the gene were selected for overexpression. Selected genes were PCR amplified from the genomic DNA of the wild type QM6a strain (ATCC13631) and cloned into a pBKT030 (FIG. 1) vector under control of the gpdA promoter from Aspergillus nidulans, using Seamless Cloning and Assembly Kit (Invitrogen), according to the manufacturer's protocol. The vector further comprised a cbhI terminator from T. reesei, a pyr2 marker with its native promoter and terminator for selection of T. reesei transformants, and upstream and downstream homologous recombination regions to enable targeted integration of the overexpression cassette at the selected locus in the T. reesei reporter host.

D. Transformation of T. reesei with Single Regulatory Protein Overexpression Cassettes

Linear cassettes comprising a gene coding for a regulatory protein, the gpdA promoter, the cbhI terminator, the pyr2 marker with its native promoter and terminator and approximately one (1) kb long upstream and downstream homologous recombination flanks, were PCR amplified from the set of pBKT030 based plasmids and transformed into the reporter strain. Preparation of protoplasts and transformation were carried out as described in PCT Publication No. WO2013/102674 (incorporated herein by reference in its entirety).

Integration of the expression cassettes into genome of the reporter strain was mediated using a Crispr-Cas9 technology to generate double strand breaks (DSBs) at a specific genomic position that stimulated DNA repair. In the fungal cells the DSBs were generated via in vitro assembled RNP complexes that can specifically cut T. reesei genome in the defined locus. The regulatory protein overexpression cassette was integrated in the locus together included the pyr2 marker, which served for selection of transformants. RNP complexes were formed in a 1:1 molar ratio between Cas9 nuclease EnGenSpy Cas9 NLS (New England BioLabs, Inc) and synthetic crRNAs annealed with tracrRNA according to recommendations of the supplier (Synthego, USA). For transformation, four (4) μl of RNP complex were mixed with 1-3 μg overexpression cassette amplified from the appropriate plasmid together with flanks for targeted integration in chromosome 3.

Two (2) independent transformants were streaked onto selective medium plates. Growing clones were screened for correct integration by PCR. In total, 691 strains overexpressing regulatory proteins were obtained and screened for improved characteristics.

As presented in TABLE 1 below, the T. reesei gene sequence (DNA SEQ) precedes the encoded regulatory protein sequence (Protein SEQ), e.g., the DNA sequence of SEQ ID NO: 1 encodes the regulatory protein of SEQ ID NO: 2, the DNA sequence of SEQ ID NO: 4 encodes the regulatory protein of SEQ ID NO: 5, the DNA sequence of SEQ ID NO: 7 encodes the regulatory protein of SEQ ID NO: 8, etc. As shown in TABLE 1, if DNA binding domain sequences (DBD SEQ) is/are present in the regulatory protein, the DBD sequence(s) is/are presented to the right of the regulatory protein sequence (Protein SEQ), e.g., the regulatory protein of SEQ ID NO: 2 comprises a DBD SEQ ID NO: 3, the regulatory protein of SEQ ID NO: 91 comprises a DBD of SEQ ID NO: 92 and a DBD of SEQ ID NO: 93.

TABLE 1
Trichoderma reesei gene (DNA)
Sequences Encoding Regulatory Proteins

| DNA SEQ | Protein SEQ | DBD SEQ |
|---|---|---|
| 1 | 2 | 3 |
| 4 | 5 | 6 |
| 7 | 8 | 9 |
| 10 | 11 | — |
| 12 | 13 | 14 |
| 15 | 16 | 17 |
| 18 | 19 | 20 |
| 21 | 22 | — |
| 23 | 24 | — |
| 25 | 26 | 27 |
| 28 | 29 | — |
| 30 | 31 | 32 |
| 33 | 34 | 35 |
| 36 | 37 | 38 |
| 39 | 40 | 41 |
| 42 | 43 | 44 |
| 45 | 46 | 47 |
| 48 | 49 | 50 |
| 51 | 52 | 53 |
| 54 | 55 | — |
| 56 | 57 | 58 |
| 59 | 60 | 61 |
| 62 | 63 | 64 |
| 65 | 66 | 67 |
| 68 | 69 | 70 |
| 71 | 72 | 73 |
| 74 | 75 | 76 |
| 77 | 78 | — |
| 79 | 80 | 81 |
| 82 | 83 | — |
| 84 | 85 | 86 |
| 87 | 88 | 89 |
| 90 | 91 | 92/93 |
| 94 | 95 | 96 |
| 97 | 98 | — |
| 99 | 100 | — |
| 101 | 102 | 103/104 |
| 105 | 106 | 107 |
| 108 | 109 | — |
| 110 | 111 | 112 |
| 113 | 114 | 115 |
| 116 | 117 | 118 |
| 119 | 120 | 121 |
| 122 | 123 | 124 |
| 125 | 126 | 127 |
| 128 | 129 | 130 |
| 131 | 132 | 133 |

### E. Construction and Transformation of Double Regulatory Protein Overexpression Cassette

A split pyr2 marker approach was applied to simultaneously overexpress two (2) regulatory proteins. The first part of the double-overexpression cassette was obtained from previously constructed vectors (Example 1C) in which the pyr2 marker is located downstream from the gene coding for a regulatory protein. Coding sequences for regulatory proteins were PCR amplified together with the gpdA promoter and the cbhI terminator, the region homologous to the upstream flank of integration locus and 5′ part of the pyr2 marker together with its native promoter.

Previously constructed set of pBKT030 based expression plasmids (Example 1C) were used as a template to PCR amplify the regulatory protein genes together with the gpdA promoter and the cbhI terminator, which were subsequently cloned into pI vector (described in PCT Publication No. WO2021/102238), downstream of the pyr2 marker. Cloning was done with Seamless Cloning and Assembly Kit (Invitrogen) according to the manufacturer's protocol.

The second part of the double-overexpression cassette was obtained from the set of pI derivative vectors comprising genes coding for regulatory proteins. Coding sequences of the regulatory proteins together with the gpdA promoter and the cbh1 terminator were amplified together with the region homologous to the downstream flank of integration locus and 3′ part of the pyr2 marker and its native terminator.

Two (2) separate parts of the double-overexpression cassette, each comprising one gene coding for a single regulatory protein were transformed in different combinations into reporter strain as described in Example 1D. Two (2) transformants per each combination were streaked onto selective medium plates. Growing clones were analysed for correct integration by PCR. In total, 134 strains overexpressing two identical or two different regulatory proteins were obtained and screened for improved characteristics as described in the Examples below.

### Example 2

### Preparation of Inoculum, Protein Production and Preparation of Samples

Ten (10) μL of spore suspension were inoculated in twenty-four (24) well polystyrene plates (Corning® Costart® CLS3527) with 1 mL of production medium (7 g/L (NH₄)₂SO₄, 4.7 g/L KH₂PO4, 1 g/L MgSO₄·7H₂O, 0.6 g/L CaCl₂·2H₂O, 33 g/L PIPPS buffer, at pH 5.5, 0.25% T. reesei trace elements; 175 g/L C₆H₈O₇, 200 g/L FeSO₄·7H₂O, 16 g/L ZnSO₄·7H₂O, 3.2 g/L CuSO₄·5H₂O, 1.4 g/L MnSO₄—H₂O and 0.8 g/L H₃BO₃) with 1.6% (w/w) glucose as a carbon source. Plates were incubated at 28° C., 200 rpm (50 mm throw) with 80% humidity for twenty-four (24) hours. One hundred (100) μL of culture solution was used for inoculation of production plates.

T. reesei production cultures were grown in twenty-four (24) well polystyrene plates (Corning® Costart® CLS3527) or plates configured such as to release lactose from a solid, porous matrix. Each well contained 1.2 mL of the production medium. In the case of polystyrene plates, a glucose/sophorose mixture at a final concentration of 2% (w/w) were used as a carbon source. In the case of lactose releasing plates, 1.6% (w/w) glucose was added to the production media. The plates were incubated at 28° C. or 34° C., 200 rpm (50 mm throw) with 80% humidity.

Sampling occurred after 120 hours of incubation. Eight hundred (800) μL of culture was collected and filtered with 0.45 μM PALL AcroPrep 96-well filter plates. Obtained supernatants were used for total protein and enzymatic activity measurements.

### Example 3

### Enzymatic Activity Measurements

The concentration of total protein in the culture supernatants was determined by BioRad Bradford assay using bovine serum albumin (BSA) as a standard.

Alpha-amylase activity in the culture supernatants was measured using α-Amylase Assay Kit (Ceralpha Method, Megazyme) according to the manufacturer's protocol.

Para-nitrophenyl phosphate (PNPP) from ThermoFisher Scientific was used as a substrate to determine phytase activity. Ten (10) mM PNPP solution was prepared in 100 mM acetate buffer (pH 5.5) containing 0.025% (w/v) Tween-20 and 0.0075 g/L CaCl₂. Phytase activity was measured by addition of four (4) μL of appropriate supernatant dilution, prepared in 100 mM acetate buffer (pH5.5), to forty-six (46) μL of PNPP substrate solution in a ninety-six (96) well microtiter plate (Costar Flat Bottom PS 3641). The micro-titer plate was sealed and incubated at 37° C. with continuous shaking at 900 rpm for ten (10) minutes. The enzymatic reaction was stopped by the addition of forty-five (45) μL of quench buffer (2 N NaOH). Absorbance of plates (endpoint) was measured at 405 nm in a spectrophotometer.

To calculate performance index (PI), the (average) enzymatic activity measured for strain overexpressing a regulatory protein was divided by the (average) enzymatic activity measured for the reporter strain not overexpressing any of the regulatory proteins.

### Example 4

### Improved Protein Production at 28° C.

All six-hundred ninety-one (691) regulatory protein overexpression mutants described above (Example 1D) were screened for improved protein production under various conditions. The protein productivity was measured by enzymatic activity assay (e.g., see, Example 3).

More particularly, regulatory proteins whose overexpression resulted in increased enzymatic activity of one of or both reporter proteins (i.e., α-amylase and/or phytase reporters) at 28° C. in lactose releasing plates after one-hundred twenty (120) hours of incubation, are set forth below in TABLE 2, wherein the numerical values represent performance indexes (PIs) calculated for alpha-amylase enzymatic assay (α-Amylase) and phytase enzymatic assay (Phytase) relative to the control reporter strain not overexpressing any regulatory proteins.

TABLE 2
Regulatory Protein Overexpression Resulting in Increased
Protein Production at 28° C. in Lactose Releasing Plates

| DNA Sequence | Encoded | | |
|---|---|---|---|
| Overexpressed | Regulatory Protein | α-Amylase | Phytase |
| SEQ ID NO: 30 | SEQ ID NO: 31 | 1.66 | NS |
| SEQ ID NO: 101 | SEQ ID NO: 102 | 1.54 | 1.27 |
| SEQ ID NO: 94 | SEQ ID NO: 95 | 1.36 | 1.09 |
| SEQ ID NO: 68 | SEQ ID NO: 69 | 1.35 | 1.15 |
| SEQ ID NO: 113 | SEQ ID NO: 114 | 1.34 | 1.28 |
| SEQ ID NO: 54 | SEQ ID NO: 55 | 1.30 | 1.06 |
| SEQ ID NO: 84 | SEQ ID NO: 85 | 1.29 | 1.16 |
| SEQ ID NO: 12 | SEQ ID NO: 13 | 1.29 | NS |
| SEQ ID NO: 122 | SEQ ID NO: 123 | 1.28 | 1.07 |
| SEQ ID NO: 65 | SEQ ID NO: 66 | 1.26 | NS |
| SEQ ID NO: 1 | SEQ ID NO: 2 | 1.25 | NS |
| SEQ ID NO: 116 | SEQ ID NO: 117 | 1.25 | 1.20 |
| SEQ ID NO: 99 | SEQ ID NO: 100 | 1.25 | 2.03 |
| SEQ ID NO: 39 | SEQ ID NO: 40 | 1.24 | 1.16 |
| SEQ ID NO: 59 | SEQ ID NO: 60 | 1.22 | 1.17 |
| SEQ ID NO: 33 | SEQ ID NO: 34 | 1.21 | 1.05 |
| SEQ ID NO: 25 | SEQ ID NO: 26 | 1.19 | NS |
| SEQ ID NO: 131 | SEQ ID NO: 132 | 1.19 | NS |
| SEQ ID NO: 105 | SEQ ID NO: 106 | 1.18 | 1.13 |
| SEQ ID NO: 56 | SEQ ID NO: 57 | 1.18 | 1.18 |
| SEQ ID NO: 15 | SEQ ID NO: 16 | 1.17 | 1.10 |
| SEQ ID NO: 4 | SEQ ID NO: 5 | 1.16 | 1.22 |
| SEQ ID NO: 87 | SEQ ID NO: 88 | 1.15 | NS |
| SEQ ID NO: 62 | SEQ ID NO: 63 | 1.15 | NS |
| SEQ ID NO: 74 | SEQ ID NO: 75 | 1.13 | NS |
| SEQ ID NO: 23 | SEQ ID NO: 24 | 1.12 | 1.36 |
| SEQ ID NO: 110 | SEQ ID NO: 111 | 1.20 | NS |
| SEQ ID NO: 18 | SEQ ID NO: 19 | 1.08 | 1.35 |
| SEQ ID NO: 48 | SEQ ID NO: 49 | 1.21 | 1.23 |
| SEQ ID NO: 128 | SEQ ID NO: 129 | 1.40 | 1.13 |
| SEQ ID NO: 97 | SEQ ID NO: 98 | 1.33 | 1.12 |

NS; not statistically significantly different from the reference

For example, as shown in TABLE 2, the mutant (recombinant) cells overexpressing a T. reesei gene (DNA) sequence comprising SEQ ID NO: 1, 4, 12, 15, 18, 23, 25, 30, 33, 39, 48, 54, 56, 59, 62, 65, 68, 74, 84, 87, 94, 97, 99, 101, 105, 110, 113, 116, 122, 128 or 131 (i.e., encoding a regulatory protein comprising SEQ ID NO: 2, 5, 13, 16, 19, 24, 26, 31, 34, 40, 49, 55, 57, 60, 63, 66, 69, 75, 85, 88, 95, 98, 100, 102, 106, 111, 114, 117, 123, 129 or 132, respectively), demonstrate enhanced protein productivity phenotypes (i.e., relative to the parental/control cells) when fermented at 28° C. under lactose releasing conditions. More particularly, such recombinant cells overexpressing the aforementioned regulatory proteins (TABLE 1) produced increased amounts of the alpha-amylase and/or phytase reporter proteins under such lactose releasing conditions (relative to control cells).

### Example 5

### Improved Protein Production at 34° C.

In the instant example, Applicant screened a subset of the six-hundred ninety-one (691) regulatory protein overexpression mutants to determine if any of the overexpressed regulatory proteins further resulted in improved protein production at a higher cultivation temperature of 34° C. The protein productivity was measured by enzymatic activity assay (e.g., see, Example 3).

For example, regulatory proteins whose overexpression resulted in increased enzymatic activity of alpha-amylase and/or phytase reporter proteins at 34° C. in lactose releasing plates after one-hundred twenty (120) hours of incubation, are set forth in TABLE 3, wherein the numerical values represent performance indexes (PIs) calculated for alpha-amylase enzymatic assay (α-Amylase) and phytase enzymatic assay (Phytase) relative to the control reporter strain not overexpressing any regulatory proteins.

TABLE 3
Regulatory Protein Overexpression Resulting in Increased
Protein Production at 34° C. in Lactose Releasing Plates

| DNA | Encoded | | |
|---|---|---|---|
| Sequence | Regulatory | | |
| Overexpressed | Protein | Amylase | Phytase |
| SEQ ID NO: 23 | SEQ ID NO: 24 | 5.27 | 2.36 |
| SEQ ID NO: 68 | SEQ ID NO: 69 | 4.43 | 4.88 |
| SEQ ID NO: 7 | SEQ ID NO: 8 | 2.59 | NS |
| SEQ ID NO: 101 | SEQ ID NO: 102 | 2.57 | 1.68 |
| SEQ ID NO: 99 | SEQ ID NO: 100 | 2.43 | 3.00 |
| SEQ ID NO: 65 | SEQ ID NO: 66 | 2.39 | 1.37 |
| SEQ ID NO: 51 | SEQ ID NO: 52 | 2.18 | 1.19 |
| SEQ ID NO: 21 | SEQ ID NO: 22 | 1.81 | 1.46 |
| SEQ ID NO: 10 | SEQ ID NO: 11 | 1.79 | 1.29 |
| SEQ ID NO: 113 | SEQ ID NO: 114 | 1.71 | 1.70 |
| SEQ ID NO: 18 | SEQ ID NO: 19 | 1.65 | 1.41 |
| SEQ ID NO: 82 | SEQ ID NO: 83 | 1.63 | NS |
| SEQ ID NO: 30 | SEQ ID NO: 31 | 1.56 | 1.20 |
| SEQ ID NO: 116 | SEQ ID NO: 117 | 1.42 | 1.26 |
| SEQ ID NO: 125 | SEQ ID NO: 126 | 1.37 | 1.16 |
| SEQ ID NO: 4 | SEQ ID NO: 5 | 1.32 | 1.89 |
| SEQ ID NO: 45 | SEQ ID NO: 46 | 1.24 | 1.22 |
| SEQ ID NO: 108 | SEQ ID NO: 109 | 1.24 | NS |
| SEQ ID NO: 42 | SEQ ID NO: 43 | 1.23 | 1.51 |
| SEQ ID NO: 15 | SEQ ID NO: 16 | 1.17 | 1.13 |
| SEQ ID NO: 122 | SEQ ID NO: 123 | 1.16 | 1.24 |
| SEQ ID NO: 79 | SEQ ID NO: 80 | 1.14 | 1.08 |
| SEQ ID NO: 105 | SEQ ID NO: 106 | 1.12 | NS |
| SEQ ID NO: 77 | SEQ ID NO: 78 | 1.11 | NS |
| SEQ ID NO: 28 | SEQ ID NO: 29 | 1.10 | NS |
| SEQ ID NO: 56 | SEQ ID NO: 57 | 1.09 | 1.13 |
| SEQ ID NO: 119 | SEQ ID NO: 120 | 1.05 | 1.10 |
| SEQ ID NO: 128 | SEQ ID NO: 129 | NS | 2.44 |
| SEQ ID NO: 54 | SEQ ID NO: 55 | NS | 1.19 |
| SEQ ID NO: 71 | SEQ ID NO: 72 | NS | 1.14 |
| SEQ ID NO: 84 | SEQ ID NO: 85 | NS | 1.13 |
| SEQ ID NO: 94 | SEQ ID NO: 95 | NS | 1.12 |
| SEQ ID NO: 90 | SEQ ID NO: 91 | NS | 1.12 |
| SEQ ID NO: 74 | SEQ ID NO: 75 | NS | 1.12 |
| SEQ ID NO: 36 | SEQ ID NO: 37 | NS | 1.09 |

NS; not statistically significantly different from the reference.

For example, as shown in TABLE 3, the mutant (recombinant) cells overexpressing a T. reesei gene (DNA) sequence comprising SEQ ID NO: 4, 7, 10, 15, 18, 21, 23, 28, 30, 36, 42, 45, 51, 54, 56, 65, 68, 71, 74, 77, 79, 82, 84, 90, 94, 99, 101, 105, 108, 113, 116, 119, 122, 125 or 128 (i.e., encoding a regulatory protein comprising SEQ ID NO: SEQ ID NO: 5, 8, 11, 16, 19, 22, 24, 29, 31, 37, 43, 46, 52, 55, 57, 66, 69, 72, 75, 78, 80, 83, 85, 91, 95, 100, 102, 106, 109, 114, 117, 120, 123, 126 or 129, respectively), demonstrate enhanced protein productivity phenotypes (i.e., relative to the parental/control cells) when fermented at 34° C. under lactose releasing conditions. More particularly, such recombinant cells overexpressing the aforementioned regulatory proteins (TABLE 3) produced increased amounts of the alpha-amylase and/or phytase reporter proteins under such lactose releasing conditions (relative to the control cells).

### Example 6

### Simultaneous Overexpression of Two Regulatory Proteins Resulting in Improved Protein Production at 28° C.

In the present example, one-hundred thirty-four (134) recombinant strains each overexpressing two different regulatory proteins, or two of the same regulatory proteins (Example 1E), were screened for improved protein production under various conditions. The protein productivity was measured by enzymatic activity assay (e.g., see, Example 3).

More particularly, combinations of two regulatory proteins whose overexpression resulted in increased enzymatic activity of the alpha-amylase reporter protein at 28° C. in lactose releasing plates after one-hundred twenty (120) hours of incubation, are set forth below in TABLE 4, wherein the numerical values represent performance indexes (PIs) calculated for alpha-amylase enzymatic assay (α-Amylase) relative to the control reporter strain not overexpressing any regulatory proteins.

TABLE 4
Simultaneous Overexpression of Two Regulatory Proteins Resulting
in Increased Protein Production at 28° C. in Lactose Releasing Plates

| Two DNA | Regulatory Protein | |
|---|---|---|
| Sequences | Combination | |
| Overexpressed | Expressed | α-Amylase |
| SEQ ID NO: 65 + 68 | SEQ ID NO: 66 + 69 | 2.07 |
| SEQ ID NO: 65 + 12 | SEQ ID NO: 66 + 13 | 2.02 |
| SEQ ID NO: 99 + 68 | SEQ ID NO: 100 + 69 | 1.99 |
| SEQ ID NO: 25 + 68 | SEQ ID NO: 26 + 69 | 1.88 |
| SEQ ID NO: 65 + 97 | SEQ ID NO: 66 + 98 | 1.80 |
| SEQ ID NO: 65 + 30 | SEQ ID NO: 66 + 31 | 1.78 |
| SEQ ID NO: 25 + 30 | SEQ ID NO: 26 + 31 | 1.73 |
| SEQ ID NO: 30 + 99 | SEQ ID NO: 31 + 100 | 1.71 |
| SEQ ID NO: 110 + 68 | SEQ ID NO: 111 + 69 | 1.71 |
| SEQ ID NO: 23 + 113 | SEQ ID NO: 24 + 114 | 1.70 |
| SEQ ID NO: 128 + 94 | SEQ ID NO: 129 + 95 | 1.70 |
| SEQ ID NO: 30 + 94 | SEQ ID NO: 31 + 95 | 1.64 |
| SEQ ID NO: 65 + 99 | SEQ ID NO: 66 + 100 | 1.62 |
| SEQ ID NO: 30 + 30 | SEQ ID NO: 31 + 31 | 1.62 |
| SEQ ID NO: 97 + 30 | SEQ ID NO: 98 + 31 | 1.60 |
| SEQ ID NO: 65 + 94 | SEQ ID NO: 66 + 95 | 1.59 |
| SEQ ID NO: 25 + 101 | SEQ ID NO: 26 + 102 | 1.58 |
| SEQ ID NO: 23 + 12 | SEQ ID NO: 24 + 13 | 1.57 |
| SEQ ID NO: 12 + 68 | SEQ ID NO: 13 + 69 | 1.56 |
| SEQ ID NO: 97 + 12 | SEQ ID NO: 98 + 13 | 1.56 |
| SEQ ID NO: 30 + 68 | SEQ ID NO: 31 + 69 | 1.55 |
| SEQ ID NO: 23 + 94 | SEQ ID NO: 24 + 95 | 1.55 |
| SEQ ID NO: 110 + 30 | SEQ ID NO: 111 + 31 | 1.53 |
| SEQ ID NO: 30 + 12 | SEQ ID NO: 31 + 13 | 1.52 |
| SEQ ID NO: 23 + 97 | SEQ ID NO: 24 + 98 | 1.52 |
| SEQ ID NO: 65 + 65 | SEQ ID NO: 66 + 66 | 1.51 |
| SEQ ID NO: 4 + 30 | SEQ ID NO: 5 + 31 | 1.51 |
| SEQ ID NO: 30 + 97 | SEQ ID NO: 31 + 98 | 1.48 |
| SEQ ID NO: 65 + 4 | SEQ ID NO: 66 + 5 | 1.48 |
| SEQ ID NO: 30 + 23 | SEQ ID NO: 31 + 24 | 1.46 |
| SEQ ID NO: 99 + 97 | SEQ ID NO: 100 + 98 | 1.45 |
| SEQ ID NO: 4 + 94 | SEQ ID NO: 5 + 95 | 1.44 |
| SEQ ID NO: 94 + 68 | SEQ ID NO: 95 + 69 | 1.44 |
| SEQ ID NO: 23 + 99 | SEQ ID NO: 24 + 100 | 1.44 |
| SEQ ID NO: 128 + 12 | SEQ ID NO: 129 + 13 | 1.42 |
| SEQ ID NO: 30 + 65 | SEQ ID NO: 31 + 66 | 1.42 |
| SEQ ID NO: 23 + 101 | SEQ ID NO: 24 + 102 | 1.41 |
| SEQ ID NO: 97 + 97 | SEQ ID NO: 98 + 98 | 1.41 |
| SEQ ID NO: 12 + 30 | SEQ ID NO: 13 + 31 | 1.40 |
| SEQ ID NO: 99 + 30 | SEQ ID NO: 100 + 31 | 1.40 |
| SEQ ID NO: 128 + 101 | SEQ ID NO: 129 + 102 | 1.40 |
| SEQ ID NO: 97 + 68 | SEQ ID NO: 98 + 69 | 1.39 |
| SEQ ID NO: 110 + 65 | SEQ ID NO: 111 + 66 | 1.37 |
| SEQ ID NO: 97 + 99 | SEQ ID NO: 98 + 100 | 1.37 |
| SEQ ID NO: 4 + 99 | SEQ ID NO: 5 + 100 | 1.35 |
| SEQ ID NO: 99 + 65 | SEQ ID NO: 100 + 66 | 1.35 |
| SEQ ID NO: 128 + 97 | SEQ ID NO: 129 + 98 | 1.34 |
| SEQ ID NO: 25 + 94 | SEQ ID NO: 26 + 95 | 1.33 |
| SEQ ID NO: 4 + 97 | SEQ ID NO: 5 + 98 | 1.32 |
| SEQ ID NO: 25 + 12 | SEQ ID NO: 26 + 13 | 1.31 |
| SEQ ID NO: 87 + 30 | SEQ ID NO: 88 + 31 | 1.31 |
| SEQ ID NO: 97 + 4 | SEQ ID NO: 98 + 5 | 1.24 |
| SEQ ID NO: 97 + 23 | SEQ ID NO: 98 + 24 | 1.23 |
| SEQ ID NO: 97 + 94 | SEQ ID NO: 98 + 95 | 1.21 |
| SEQ ID NO: 25 + 65 | SEQ ID NO: 26 + 66 | 1.20 |
| SEQ ID NO: 94 + 30 | SEQ ID NO: 95 + 31 | 1.20 |
| SEQ ID NO: 23 + 4 | SEQ ID NO: 24 + 5 | 1.18 |
| SEQ ID NO: 12 + 12 | SEQ ID NO: 13 + 13 | 1.12 |
| SEQ ID NO: 94 + 94 | SEQ ID NO: 95 + 95 | 1.12 |

As shown in TABLE 4, the mutant (recombinant) cells simultaneously overexpressing the T. reesei gene (DNA) sequence combinations of SEQ ID NO: 4 and 30, SEQ ID NO: 4 and 94, SEQ ID NO: 4 and 97, SEQ ID NO: 4 and 99, SEQ ID NO: 12 and 12, SEQ ID NO: 12 and 30, SEQ ID NO: 12 and 68, SEQ ID NO: 23 and 101, SEQ ID NO: 23 and 113, SEQ ID NO: 23 and 12, SEQ ID NO: 23 and 4, SEQ ID NO: 23 and 94, SEQ ID NO: 23 and 97, SEQ ID NO: 23 and 99. SEQ ID NO: 25 and 101, SEQ ID NO: 25 and 12, SEQ ID NO: 25 and 30, SEQ ID NO: 25 and 65, SEQ ID NO: 25 and 68, SEQ ID NO: 25 and 94, SEQ ID NO: 30 and 12, SEQ ID NO: 30 and 23, SEQ ID NO: 30 and 30, SEQ ID NO: 30 and 65, SEQ ID NO: 30 and 68, SEQ ID NO: 30 and 94. SEQ ID NO: 30 and 97, SEQ ID NO: 30 and 99, SEQ ID NO: 65 and 12, SEQ ID NO: 65 and 30, SEQ ID NO: 65 and 4, SEQ ID NO: 65 and 65, SEQ ID NO: 65 and 68, SEQ ID NO: 65 and 94, SEQ ID NO: 65 and 97, SEQ ID NO: 65 and 99, SEQ ID NO: 87 and 30, SEQ ID NO: 94 and 30, SEQ ID NO: 94 and 68, SEQ ID NO: 94 and 94, SEQ ID NO: 97 and 12, SEQ ID NO: 97 and 23, SEQ ID NO: 97 and 30, SEQ ID NO: 97 and 4, SEQ ID NO: 97 and 68, SEQ ID NO: 97 and 94, SEQ ID NO: 97 and 97, SEQ ID NO: 97 and 99, SEQ ID NO: 99 and 30, SEQ ID NO: 99 and 65, SEQ ID NO: 99 and 68, SEQ ID NO: 99 and 97, SEQ ID NO: 110 and 30, SEQ ID NO: 110 and 65, SEQ ID NO: 110 and 68, SEQ ID NO: 128 and 101, SEQ ID NO: 128 and 12, SEQ ID NO: 128 and 94, or SEQ ID NO: 128 and 97, demonstrate enhanced protein productivity phenotypes (i.e., relative to the parental/control cells) when fermented at 28° C. under lactose releasing conditions. More particularly, such recombinant cells overexpressing the aforementioned combinations of two regulatory proteins (TABLE 4) produced increased amounts of alpha-amylase reporter under such lactose releasing conditions (relative to the control cells).

Likewise, combinations of two regulatory proteins whose overexpression resulted in increased production of the alpha-amylase reporter protein at 28° C. in polystyrene plates with 2% (w/w) glucose/sophorose as a carbon source after one-hundred twenty (120) hours of incubation, are set forth below in TABLE 5, wherein the numerical values represent performance indexes (PIs) calculated for alpha-amylase enzymatic assay (α-Amylase) relative to the control reporter strain not overexpressing any regulatory proteins.

TABLE 5
Simultaneous Overexpression of Two Regulatory
Proteins Resulting in Increased Protein Production at 28° C.
with 2% (w/w) Glucose/Sophorose as Carbon Source

| | Regulatory Protein | |
|---|---|---|
| Two DNA Sequences | Combination | |
| Overexpressed | Expressed | α-Amylase |
| SEQ ID NO: 99 + 68 | SEQ ID NO: 100 + 69 | 1.91 |
| SEQ ID NO: 99 + 101 | SEQ ID NO: 100 + 102 | 1.58 |
| SEQ ID NO: 12 + 99 | SEQ ID NO: 13 + 100 | 1.52 |
| SEQ ID NO: 99 + 65 | SEQ ID NO: 100 + 66 | 1.50 |
| SEQ ID NO: 94 + 68 | SEQ ID NO: 95 + 69 | 1.49 |
| SEQ ID NO: 99 + 97 | SEQ ID NO: 100 + 98 | 1.44 |
| SEQ ID NO: 99 + 12 | SEQ ID NO: 100 + 13 | 1.44 |
| SEQ ID NO: 30 + 101 | SEQ ID NO: 31 + 102 | 1.41 |
| SEQ ID NO: 94 + 99 | SEQ ID NO: 95 + 100 | 1.37 |
| SEQ ID NO: 99 + 23 | SEQ ID NO: 100 + 24 | 1.34 |
| SEQ ID NO: 99 + 94 | SEQ ID NO: 100 + 95 | 1.32 |
| SEQ ID NO: 25 + 101 | SEQ ID NO: 26 + 102 | 1.27 |
| SEQ ID NO: 97 + 113 | SEQ ID NO: 98 + 114 | 1.27 |
| SEQ ID NO: 30 + 12 | SEQ ID NO: 31 + 13 | 1.27 |
| SEQ ID NO: 110 + 101 | SEQ ID NO: 111 + 102 | 1.26 |
| SEQ ID NO: 97 + 99 | SEQ ID NO: 98 + 100 | 1.25 |
| SEQ ID NO: 30 + 23 | SEQ ID NO: 31 + 24 | 1.24 |
| SEQ ID NO: 30 + 94 | SEQ ID NO: 31 + 95 | 1.24 |
| SEQ ID NO: 97 + 101 | SEQ ID NO: 98 + 102 | 1.24 |
| SEQ ID NO: 94 + 4 | SEQ ID NO: 95 + 5 | 1.23 |
| SEQ ID NO: 12 + 23 | SEQ ID NO: 13 + 24 | 1.22 |
| SEQ ID NO: 94 + 12 | SEQ ID NO: 95 + 13 | 1.21 |
| SEQ ID NO: 94 + 94 | SEQ ID NO: 95 + 95 | 1.21 |
| SEQ ID NO: 99 + 99 | SEQ ID NO: 100 + 100 | 1.20 |
| SEQ ID NO: 99 + 30 | SEQ ID NO: 100 + 31 | 1.19 |
| SEQ ID NO: 30 + 97 | SEQ ID NO: 31 + 98 | 1.19 |
| SEQ ID NO: 94 + 101 | SEQ ID NO: 95 + 102 | 1.19 |
| SEQ ID NO: 87 + 99 | SEQ ID NO: 88 + 100 | 1.16 |
| SEQ ID NO: 12 + 65 | SEQ ID NO: 13 + 66 | 1.16 |
| SEQ ID NO: 97 + 30 | SEQ ID NO: 98 + 31 | 1.15 |
| SEQ ID NO: 12 + 101 | SEQ ID NO: 13 + 102 | 1.15 |
| SEQ ID NO: 30 + 65 | SEQ ID NO: 31 + 66 | 1.14 |
| SEQ ID NO: 12 + 128 | SEQ ID NO: 13 + 129 | 1.12 |
| SEQ ID NO: 94 + 30 | SEQ ID NO: 95 + 31 | 1.12 |
| SEQ ID NO: 94 + 23 | SEQ ID NO: 95 + 24 | 1.11 |
| SEQ ID NO: 87 + 113 | SEQ ID NO: 88 + 114 | 1.10 |

Thus, as presented above in TABLE 5, the mutant (recombinant) cells simultaneously overexpressing a T. reesei gene (DNA) sequence combination of SEQ ID NO: 12 and 101, SEQ ID NO: 12 and 128, SEQ ID NO: 12 and 23, SEQ ID NO: 12 and 65, SEQ ID NO: 12 and 99, SEQ ID NO: 25 and 101, SEQ ID NO: 30 and 101, SEQ ID NO: 30 and 12, SEQ ID NO: 30 and 23, SEQ ID NO: 30 and 65, SEQ ID NO: 30 and 94, SEQ ID NO: 30 and 97, SEQ ID NO: 87 and 113, SEQ ID NO: 87 and 99, SEQ ID NO: 94 and 101, SEQ ID NO: 94 and 12, SEQ ID NO: 94 and 23, SEQ ID NO: 94 and 30, SEQ ID NO: 94 and 4, SEQ ID NO: 94 and 68, SEQ ID NO: 94 and 94, SEQ ID NO: 94 and 99, SEQ ID NO: 97 and 101, SEQ ID NO: 97 and 113, SEQ ID NO: 97 and 30, SEQ ID NO: 97 and 99, SEQ ID NO: 99 and 101, SEQ ID NO: 99 and 12, SEQ ID NO: 99 and 23, SEQ ID NO: 99 and 30, SEQ ID NO: 99 and 65, SEQ ID NO: 99 and 68, SEQ ID NO: 99 and 94, SEQ ID NO: 99 and 97, SEQ ID NO: 99 and 99, or SEQ ID NO: 110 and 101, demonstrate an enhanced protein productivity phenotype (i.e., relative to the parental/control cells) when fermented at 28° C. with 2% (w/w) glucose/sophorose as a carbon source. For example, recombinant cells overexpressing the aforementioned combinations of two regulatory proteins (TABLE 5) produced increased amounts of the alpha-amylase reporter under such glucose/sophorose conditions (relative to parental cells).

### Example 7

### Protein Production in Small Scale Fed-Batch Fermentation

A subset of T. reesei strains overexpressing a single regulatory protein (Examples 4 and 5) or two regulatory proteins (Example 6) were evaluated in two (2) L bioreactors (in a fed-batch fermentation), using sophorose as an inducing substrate at a constant feed rate. More particularly, the T. reesei fermentation was carried out as generally described in U.S. Pat. No. 7,713,725 (incorporated herein by reference in its entirety) using seed cultures in citrate minimal medium. During fermentation, the supernatant from all cultures was harvested at different time points and analyzed by an enzymatic activity assay, as described above in Example 3.

### Example 8

### Improved Protein Production in Fermentation by Overexpression of Regulatory Proteins

The T. reesei mutant (recombinant) strains overexpressing the regulatory protein of SEQ ID NO: 31, SEQ ID NO: 66, SEQ ID NO: 69, or SEQ ID NO: 102, were further evaluated in small scale fed-batch fermentations, as described in Example 4. For example, FIG. 2 represents the enzyme production of the recombinant strains relative to the control reporter strain (not overexpressing any regulatory proteins), throughout the fermentation, wherein protein productivity was measured by enzymatic activity assay as described above in Example 3. More particularly, as shown in FIG. 2, all of the mutant strains evaluated exhibited improved protein production.

### Example 9

### Improved Protein Production in Fermentation, by Simultaneous Overexpression of Two Regulatory Proteins

The T. reesei mutant (recombinant) strains overexpressing the regulatory protein combinations of SEQ ID NO: 66 and SEQ ID NO: 69, SEQ ID NO: 31 and SEQ ID NO: 66, SEQ ID NO: 31 and SEQ ID NO: 69, SEQ ID NO: 100 and SEQ ID NO: 69, or SEQ ID NO: 100 and SEQ ID NO: 66, were evaluated in small scale fed-batch fermentations, as described in Example 7. More particularly, FIG. 3 represents the enzyme production of the mutant strains, based on the enzymatic activity assay (e.g., see, Example 3), compared to the control reporter strain not overexpressing any regulatory proteins. As shown in FIG. 3, the five evaluated mutant (recombinant) strains exhibited higher protein productivity than the control strain throughout the fermentation.

### Example 10

### Fungal Regulatory Protein Orthologues

In the instant example Applicant has performed a homology based search of SEQ ID NO: 2, SEQ ID NO: 5, SEQ ID NO: 8, SEQ ID NO: 11, SEQ ID NO: 13, SEQ ID NO: 16, SEQ ID NO: 19, SEQ ID NO: 22, SEQ ID NO: 24, SEQ ID NO: 26, SEQ ID NO: 29, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 52, SEQ ID NO: 55, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 78, SEQ ID NO: 80, SEQ ID NO: 83, SEQ ID NO: 85, SEQ ID NO: 88, SEQ ID NO: 91, SEQ ID NO: 95, SEQ ID NO: 98, SEQ ID NO: 100, SEQ ID NO: 102, SEQ ID NO: 106, SEQ ID NO: 109, SEQ ID NO: 111, SEQ ID NO: 114, SEQ ID NO: 117, SEQ ID NO: 120, SEQ ID NO: 123, SEQ ID NO: 126, SEQ ID NO: 129, and SEQ ID NO: 132 against fungal species Trichoderma reesei (GCF_000167675.1), Thermothelomyces thermophilus (GCF_000226095.1), Aspergillus oryzae (GCF_000184455.2), Yarrowia lipolytica (GCF_000002525.2), Komagataella pastoris (GCA_001708105.1), Saccharomyces cerevisiae (GCF_000146045.2), Schizosaccharomyces pombe (GCF_000002945.1), and Aspergillus niger (GCF_000002855.3). As shown below, TABLE 6 lists orthologs identified for these selected group of fungal species.

TABLE 6
REGULATORY PROTEIN ORTHOLOGUES

| SEQ ID | Gene ID | % Identity | Organism |
|---|---|---|---|
| 2 | XP_006960955.1 | 100.00 | Trichoderma reesei QM6a |
| 2 | XP_003661468.1 | 55.68 | Thermothelomyces thermophilus |
| | | | ATCC 42464 |
| 5 | XP_006964374.1 | 100.00 | Trichoderma reesei QM6a |
| 5 | XP_003661571.1 | 60.79 | Thermothelomyces thermophilus |
| | | | ATCC 42464 |
| 5 | XP_001824144.3 | 40.34 | Aspergillus oryzae RIB40 |
| 8 | XP_006968920.1 | 100.00 | Thrichoderma reesei QM6a |
| 8 | XP_003661341.1 | 57.04 | Thermothelomyces thermophilus |
| | | | ARCC 42464 |
| 8 | XP_001727578.1 | 43.48 | Aspergillus oryzae RIB40 |
| 8 | XP_500513.1 | 42.41 | Yarrowia lipolytica CLIB122 |
| 8 | XP_006965329.1 | 40.58 | Trichoderma reesei QM6a |
| 8 | XP_001392717.2 | 40.08 | Aspergillus niger CBS 513.88 |
| 11 | XP_006966212.1 | 100.00 | Trichoderma reesei QM6a |
| 13 | XP_006970024.1 | 100.00 | Trichoderma reesei QM6a |
| 13 | XP_001398851.2 | 40.41 | Aspergillus niger CBS 513.88 |
| 16 | XP_006965249.1 | 100.00 | Trichoderma reesei QM6a |
| 16 | XP_003660806.1 | 43.32 | Thermothelomyces thermophilus |
| | | | ATCC 42464 |
| 16 | XP_006961090.1 | 40.15 | Trichoderma reesei QM6a |
| 19 | XP_006962017.1 | 100.00 | Trichoderma reesei QM6a |
| 19 | XP_003660584.1 | 53.06 | Thermothelomyces thermophilus |
| | | | ATCC 42464 |
| 19 | XP_502545.1 | 47.90 | Yarrowia lipolytica CLIB122 |
| 19 | XP_001818743.3 | 47.34 | Aspergillus oryzae RIB40 |
| 19 | XP_001400926.2 | 46.01 | Aspergillus niger CBS 513.88 |
| 19 | XP_023090801.1 | 43.61 | Aspergillus oryzae RIB40 |
| 19 | XP_001819331.1 | 43.16 | Aspergillus oryzae RIB40 |
| 19 | XP_003659353.1 | 42.86 | Thermothelomyces thermophilus |
| | | | ATCC 42464 |
| 19 | XP_006963406.1 | 42.11 | Trichoderma reesei QM6a |
| 19 | XP_003664110.1 | 42.07 | Thermothelomyces thermophilus |
| | | | ATCC 42464 |
| 19 | XP_023092543.1 | 41.84 | Aspergillus oryzae RIB40 |
| 19 | XP_003659550.1 | 41.61 | Thermothelomyces thermophilus |
| | | | ATCC 42464 |
| 19 | XP_504094.1 | 41.60 | Yarrowia lipolytica CLIB122 |
| 19 | XP_003189187.2 | 41.59 | Aspergillus oryzae RIB40 |
| 19 | XP_003662017.1 | 41.38 | Thermothelomyces thermophilus |
| | | | ATCC 42464 |
| 19 | XP_003659697.1 | 41.28 | Thermothelomyces thermophilus |
| | | | ATCC 42464 |
| 19 | XP_001401034.1 | 41.12 | Aspergillus niger CBS 513.88 |
| 19 | NP_011784.3 | 40.35 | Saccharomyces cerevisiae S288C |
| 19 | XP_003658723.1 | 40.34 | Thermothelomyces thermophilus |
| | | | ATCC 42464 |
| 19 | XP_001399501.2 | 40.32 | Aspergillus niger CBS 513.88 |
| 19 | XP_006965661.1 | 40.11 | Trichoderma reesei QM6a |
| 19 | XP_001820784.1 | 40.00 | Aspergillus oryzae RIB40 |
| 19 | XP_499816.1 | 40.00 | Yarrowia lipolytica CLIB122 |
| 22 | XP_006961125.1 | 100.00 | Trichoderma reesei QM6a |
| 22 | XP_023092994.1 | 62.82 | Aspergillus oryzae RIB40 |
| 22 | XP_006961035.1 | 52.31 | Trichoderma reesei QM6a |
| 22 | XP_001400256.2 | 47.93 | Aspergillus niger CBS 513.88 |
| 22 | XP_003659674.1 | 40.09 | Thermothelomyces thermophilus |
| | | | ATCC 42464 |
| 24 | XP_006961502.1 | 100.00 | Thricoderma reesei QM6a |
| 24 | XP_003662399.1 | 57.68 | Thermothelomyces thermophilus |
| | | | ATCC 42464 |
| 24 | XP_001394108.2 | 49.23 | Aspergillus niger CBS 513.88 |
| 24 | XP_001820832.1 | 48.28 | Aspergillus oryzae RIB40 |
| 24 | XP_001396751.1 | 47.47 | Aspergillus niger CBS 513.88 |
| 24 | XP_003188808.1 | 45.28 | Aspergillus niger CBS 513.88 |
| 26 | XP_006962182.1 | 100.00 | Trichoderma reesei QM6a |
| 26 | XP_003660263.1 | 67.23 | Thermothelomyces thermophilus |
| | | | ATCC 42464 |
| 26 | XP_023091408.1 | 59.02 | Aspergillus oyzae RIB40 |
| 26 | XP_001389599.1 | 53.99 | Aspergillus niger CBS 513.88 |
| 26 | XP_505203.1 | 47.68 | Yarrowia lipolytica CLIB122 |
| 26 | XP_006967873.1 | 41.46 | Trichoderma reesei QM6a |
| 26 | NP_010185.1 | 40.61 | Saccharomyces cerevisiae S288C |
| 26 | XP_023091818.1 | 40.24 | Aspergillus oryzae RIB40 |
| 29 | XP_006962151.1 | 100.00 | Trichoderma reesei QM6a |
| 29 | XP_003660615.1 | 62.39 | Thermothelomyces thermophilus |
| | | | ATCC 42464 |
| 29 | XP_001394895.1 | 53.72 | Aspergillus niger CBS 513.88 |
| 29 | XP_001819158.1 | 50.41 | Aspergillus oryzae RIB40 |
| 29 | NP_588059.1 | 46.15 | Schizosaccharomyces pombe |
| 29 | ANZ74907.1 | 45.00 | Komagataella pastoris |
| 29 | XP_023088856.1 | 44.93 | Aspergillus oryzae RIB40 |
| 29 | XP_499865.1 | 44.92 | Yarrowia lipolytica CLIB122 |
| 29 | XP_503585.1 | 44.59 | Yarrowia lipolytica CLIB122 |
| 29 | XP_001826422.3 | 43.75 | Aspergillus oryzae RIB40 |
| 29 | XP_002143066.1 | 43.18 | Yarrowia lipolytica CLIB122 |
| 29 | XP_006964550.1 | 42.65 | Trichoderma reesei QM6a |
| 29 | XP_001401997.1 | 42.50 | Aspergillus niger CBS 513.88 |
| 29 | XP_001822922.1 | 42.31 | Aspergillus oryzae RIB40 |
| 29 | XP_003665060.1 | 42.19 | Thermothelomyces thermophilus |
| | | | ATCC 42464 |
| 29 | XP_023089935.1 | 41.94 | Aspergillus oryzae RIB40 |
| 29 | NP_001257670.1 | 41.67 | Saccharomyces cerevisiae S288C |
| 29 | ANZ77867.1 | 41.54 | Komagataella pastoris |
| 29 | NP_010712.1 | 41.38 | Saccharomyces cerevisiae S288C |
| 29 | ANZ77059.1 | 41.25 | Komagataella pastoris |
| 29 | XP_505046.1 | 41.10 | Yarrowia lipolytica CLIB122 |
| 29 | XP_003660264.1 | 41.05 | Thermothelomyces thermophilus |
| | | | ATCC 42464 |
| 29 | XP_001395464.2 | 41.05 | Aspergillus niger CBS 513.88 |
| 29 | NP_593833.1 | 40.91 | Schizosaccharomyces pombe |
| 29 | ANZ76050.1 | 40.68 | Komagataella pastoris |
| 29 | XP_006966543.1 | 40.40 | Trichoderma reesei QM6a |
| 29 | XP_003658794.1 | 40.38 | Thermothelomyces thermophilus |
| | | | ATCC 42464 |
| 29 | NP_219498.1 | 40.38 | Saccharomyces cerevisiae S288C |
| 29 | XP_003660118.1 | 40.24 | Thermothelomyces thermophilus |
| | | | ATCC 42464 |
| 29 | XP_001400274.1 | 40.23 | Aspergillus niger CBS 513.88 |
| 29 | XP_023090657.1 | 40.18 | Aspergillus oryzae RIB40 |
| 29 | XP_001824881.1 | 40.00 | Aspergillus oryzae RIB40 |
| 29 | XP_023089888.1 | 40.00 | Aspergillus oryzae RIB40 |
| 29 | XP_003188893.1 | 40.00 | Aspergillus niger CBS 513.88 |
| 29 | XP_006968405.1 | 40.00 | Trichoderma reesei QM6a |
| 29 | NP_588490.1 | 40.00 | Schizosaccharomyces pombe |
| 29 | NP_015388.1 | 40.00 | Saccharomyces cerevisiae S288C |
| 31 | XP_006965619.1 | 100.00 | Trichoderma reesei QM6a |
| 34 | XP_006965747.1 | 100.00 | Trichoderma reesei QM6a |
| 34 | XP_003665576.1 | 59.16 | Thermothelomyces thermophilus |
| | | | ATCC 42464 |
| 34 | XP_001399002.2 | 41.74 | Aspergillus niger CBS 513.88 |
| 34 | XP_001818971.1 | 40.88 | Aspergillus oryzae RIB40 |
| 34 | XP_006965318.1 | 40.09 | Trichoderma reesei QM6a |
| 37 | XP_006965596.1 | 100.00 | Trichoderma reesei QM6a |
| 37 | XP_003665424.1 | 54.47 | Thermothelomyces thermophilus |
| | | | ATCC 42464 |
| 37 | XP_023092662.1 | 49.46 | Aspergillus oryzae RIB40 |
| 37 | XP_500925.1 | 48.52 | Yarrowia lipolytica CLIB122 |
| 37 | ANZ76822.1 | 42.45 | Komagataella pastoris |
| 40 | XP_006969385.1 | 100.00 | Trichoderma reesei QM6a |
| 40 | XP_003660985.1 | 45.94 | Thermothelomyces thermophilus |
| | | | ATCC 42464 |
| 43 | XP_006969614.1 | 100.00 | Trichoderma reesei QM6a |
| 46 | XP_006969713.1 | 100.00 | Trichoderma reesei QM6a |
| 46 | XP_003662677.1 | 44.06 | Thermothelomyces thermophilus |
| | | | ATCC 42464 |
| 46 | XP_001818108.3 | 41.35 | Aspergillus oryzae RIB40 |
| 49 | XP_006969990.1 | 100.00 | Trichoderma reesei QM6a |
| 49 | XP_006962863.1 | 50.51 | Trichoderma reesei QM6a |
| 52 | XP_006963224.1 | 100.00 | Trichoderma reesei QM6a |
| 52 | XP_003662857.1 | 61.66 | Thermothelomyces thermophilus |
| | | | ATCC 42464 |
| 52 | XP_500194.1 | 44.77 | Yarrowia lipolytica CLIB122 |
| 52 | XP_023091097.1 | 44.23 | Aspergillus oryzae RIB40 |
| 52 | XP_001391780.1 | 43.16 | Aspergillus niger CBS 513.88 |
| 52 | ANZ77983.1 | 41.09 | Komagataella pastoris |
| 55 | XP_006963728.1 | 97.51 | Trichoderma reesei QM6a |
| 55 | XP_001392006.1 | 46.40 | Aspergillus niger CBS 513.88 |
| 55 | XP_006968091.1 | 43.60 | Trichoderma reesei QM6a |
| 55 | XP_003658577.1 | 43.33 | Thermothelomyces thermophilus |
| | | | ATCC 42464 |
| 55 | XP_001727882.3 | 43.04 | Aspergillus oryzae RIB40 |
| 55 | XP_003659953.1 | 42.86 | Thermothelomyces thermophilus |
| | | | ATCC 42464 |
| 55 | XP_003666780.1 | 41.60 | Thermothelomyces thermophilus |
| | | | ATCC 42464 |
| 55 | XP_003665260.1 | 41.57 | Thermothelomyces thermophilus |
| | | | ATCC 42464 |
| 55 | XP_003664853.1 | 40.54 | Thermothelomyces thermophilus |
| | | | ATCC 42464 |
| 55 | XP_500851.1 | 40.38 | Yarrowia lipolytica CLIB122 |
| 55 | XP_001395019.1 | 40.21 | Aspergillus niger CBS 513.88 |
| 55 | XP_003663011.1 | 40.12 | Thermothelomyces thermophilus |
| | | | ATCC 42464 |
| 57 | XP_006964079.1 | 100.00 | Trichoderma reesei QM6a |
| 57 | XP_023090452.1 | 57.10 | Aspergillus oryzae RIB40 |
| 60 | XP_006964546.1 | 100.00 | Trichoderma reesei QM6a |
| 60 | XP_003661773.1 | 57.17 | Thermothelomyces thermophilus |
| | | | ATCC 42464 |
| 60 | XP_001393942.1 | 52.61 | Aspergillus niger CBS 513.88 |
| 60 | XP_003188786.1 | 52.61 | Aspergillus niger CBS 513.88 |
| 60 | XP_003190187.1 | 47.88 | Aspergillus oryzae RIB40 |
| 60 | XP_500831.1 | 42.39 | Yarrowia lipolytica CLIB122 |
| 60 | XP_503831.1 | 40.33 | Yarrowia lipolytica CLIB122 |
| 60 | ANZ73126.1 | 40.05 | Komagataella pastoris |
| 63 | XP_006964546.1 | 100.00 | Trichoderma reesei QM6a |
| 63 | XP_003661773.1 | 52.86 | Thermothelomyces thermophilus |
| | | | ATCC 42464 |
| 63 | XP_001393942.1 | 52.82 | Aspergillus niger CBS 513.88 |
| 63 | XP_003188786.1 | 52.82 | Aspergillus niger CBS 513.88 |
| 63 | XP_003190187.1 | 47.97 | Aspergillus oryzae RIB40 |
| 63 | XP_500831.1 | 41.62 | Yarrowia lipolytica CLIB122 |
| 63 | XP_501207.2 | 40.12 | Yarrowia lipolytica CLIB122 |
| 63 | ANZ73126.1 | 40.05 | Komagataella pastoris |
| 66 | XP_006967198.1 | 100.00 | Trichoderma reesei QM6a |
| 66 | XP_003664619.1 | 69.70 | Thermothelomyces thermophilis |
| | | | ATCC 42464 |
| 66 | XP_001396231.2 | 60.56 | Aspergillus niger CBS 513.88 |
| 66 | XP_023088815.1 | 60.19 | Aspergillus oryzae RIB40 |
| 66 | XP_006963776.1 | 45.91 | Trichoderma reesei QM6a |
| 69 | XP_006969776.1 | 100.00 | Trichoderma reesei QM6a |
| 69 | XP_003667088.1 | 58.06 | Thermothelomyces thermophilus |
| | | | ATCC 42464 |
| 69 | XP_023091428.1 | 48.91 | Aspergillus oryzae RIB40 |
| 69 | XP_001392800.1 | 48.18 | Aspergillus niger CBS 513.88 |
| 69 | XP_003188748.1 | 48.18 | Aspergillus niger CBS 513.88 |
| 69 | XP_001817799.3 | 43.68 | Aspergillus oryzae RIB40 |
| 69 | XP_003667125.1 | 40.93 | Thermothelomyces thermophilus |
| | | | ATCC 42464 |
| 72 | XP_006961158.1 | 100.00 | Trichoderma reesei QM6a |
| 75 | XP_006961563.1 | 99.56 | Trichoderma reesei QM6a |
| 78 | XP_006962590.1 | 100.00 | Trichoderma reesei QM6a |
| 78 | XP_006963612.1 | 40.83 | Trichoderma reesei QM6a |
| 78 | XP_001819190.1 | 40.08 | Aspergillus oryzae RIB40 |
| 80 | XP_006963801.1 | 100.00 | Trichoderma reesei QM6a |
| 83 | XP_006963882.1 | 100.00 | Trichoderma reesei QM6a |
| 83 | XP_006962319.1 | 41.63 | Trichoderma reesei QM6a |
| 83 | XP_006968053.1 | 40.00 | Trichoderma reesei QM6a |
| 85 | XP_006964092.1 | 100.00 | Trichoderma reesei QM6a |
| 85 | XP_001397013.2 | 40.50 | Aspergillus niger CBS 513.88 |
| 88 | XP_006964140.1 | 100.00 | Trichoderma reesei QM6a |
| 88 | XP_003659490.1 | 43.18 | Thermothelomyces thermophilus |
| | | | ATCC 42464 |
| 88 | XP_001393430.1 | 42.39 | Aspergillus niger CBS 513.88 |
| 88 | XP_023094158.1 | 42.14 | Aspergillus oryzae RIB40 |
| 88 | NP_596752.1 | 41.94 | Schizosaccharomyces pombe |
| 88 | XP_023093401.1 | 41.76 | Aspergillus oryzae RIB40 |
| 88 | XP_001392555.2 | 41.67 | Aspergillus niger CBS 513.88 |
| 88 | XP_006964735.1 | 41.67 | Trichoderma reesei QM6a |
| 88 | XP_003662007.1 | 40.98 | Thermothelomyces thermophilus |
| | | | ATCC 42464 |
| 88 | XP_006969632.1 | 40.70 | Trichoderma reesei QM6a |
| 88 | XP_006969126.1 | 40.65 | Trichoderma reesei QM6a |
| 88 | XP_023090751.1 | 40.40 | Aspergillus oryzae RIB40 |
| 88 | XP_001825039.1 | 40.40 | Aspergillus oryzae RIB40 |
| 91 | XP_006965880.1 | 100.00 | Trichoderma reesei QM6a |
| 91 | XP_023091064.1 | 51.69 | Aspergillus oryzae RIB40 |
| 91 | XP_006967897.1 | 44.55 | Trichoderma reesei QM6a |
| 91 | XP_023090280.1 | 43.21 | Aspergillus oryzae RIB40 |
| 91 | XP_023089680.1 | 43.14 | Aspergillus oryzae RIB40 |
| 91 | XP_001391075.2 | 42.72 | Aspergillus niger CBS 513.88 |
| 91 | XP_001822757.1 | 42.05 | Aspergillus oryzae RIB40 |
| 91 | XP_001822763.3 | 41.67 | Aspergillus oryzae RIB40 |
| 91 | XP_006966085.1 | 41.67 | Trichoderma reesei QM6a |
| 91 | XP_003661176.1 | 41.62 | Thermothelomyces thermophilus |
| | | | ATCC 42464 |
| 91 | XP_003188531.1 | 41.57 | Aspergillus niger CBS 513.88 |
| 91 | XP_023090493.1 | 41.24 | Aspergillus oryzae RIB40 |
| 91 | XP_003666224.1 | 41.03 | Thermothelomyces thermophilus |
| | | | ATCC 42464 |
| 91 | XP_006965595.1 | 41.03 | Trichoderma reesei QM6a |
| 91 | XP_001393650.1 | 40.96 | Aspergillus niger CBS 513.88 |
| 91 | XP_006964498.1 | 40.52 | Trichoderma reesei QM6a |
| 91 | XP_003188629.1 | 40.51 | Aspergillus niger CBS 513.88 |
| 91 | XP_023091412.1 | 40.43 | Aspergillus oryzae RIB40 |
| 91 | NP_013400.1 | 40.40 | Saccharomyces cerevisiae S288C |
| 91 | XP_001821926.1 | 40.38 | Aspergillus oryzae RIB40 |
| 91 | XP_023092952.1 | 40.26 | Aspergillus oryzae RIB40 |
| 91 | XP_003661293.1 | 40.00 | Thermothelomyces thermophilus |
| | | | ATCC 42464 |
| 91 | XP_501675.1 | 40.00 | Yarrowia lipolytica CLIB122 |
| 95 | XP_006967005.1 | 100.00 | Trichoderma reesei QM6a |
| 95 | XP_006962831.1 | 44.77 | Trichoderma reesei QM6a |
| 95 | XP_006968727.1 | 40.56 | Trichoderma reesei QM6a |
| 98 | XP_006967946.1 | 100.00 | Trichoderma reesei QM6a |
| 98 | XP_006969924.1 | 61.44 | Trichoderma reesei QM6a |
| 98 | XP_001402104.1 | 43.93 | Aspergillus niger CBS 513.88 |
| 98 | XP_006967221.1 | 42.22 | Trichoderma reesei QM6a |
| 98 | ANZ74388.1 | 41.32 | Komagataella pastoris |
| 98 | XP_023092293.1 | 41.28 | Aspergillus oryzae RIB40 |
| 98 | XP_002143111.1 | 40.96 | Yarrowia lipolytica CLIB122 |
| 98 | XP_003662133.1 | 40.82 | Thermothelomyces thermophilus |
| | | | ATCC 42464 |
| 98 | XP_023093875.1 | 40.71 | Aspergillus oryzae RIB40 |
| 98 | XP_006963145.1 | 40.43 | Trichoderma reesei QM6a |
| 98 | XP_006963698.1 | 40.35 | Trichoderma reesei QM6a |
| 98 | ANZ73581.1 | 40.13 | Komagataella pastoris |
| 98 | XP_023094140.1 | 40.00 | Aspergillus oryzae RIB40 |
| 98 | XP_023091891.1 | 40.00 | Aspergillus oryzae RIB40 |
| 100 | XP_006968178.1 | 100.00 | Trichoderma reesei QM6a |
| 100 | XP_023088676.1 | 40.25 | Aspergillus oryzae RIB40 |
| 102 | XP_501720.1 | 47.46 | Yarrowia lipolytica CLIB122 |
| 102 | XP_505887.1 | 47.46 | Yarrowia lipolytica CLIB122 |
| 102 | XP_001821677.1 | 45.76 | Aspergillus oryzae RIB40 |
| 102 | XP_003665244.1 | 44.93 | Thermothelomyces thermophilus |
| | | | ATCC 42464 |
| 102 | NP_594574.1 | 44.62 | Schizosaccharomyces pombe |
| 102 | NP_009563.1 | 44.44 | Saccharomyces cerevisiae S288C |
| 102 | NP_014368.1 | 44.44 | Saccharomyces cerevisiae S288C |
| 102 | XP_023092097.1 | 44.29 | Aspergillus oryzae RIB40 |
| 102 | ANZ74923.1 | 44.07 | Komagatella pastoris |
| 102 | ANZ76994.1 | 44.07 | Komagatella pastrois |
| 102 | XP_006963420.1 | 43.75 | Trichoderma reesei QM6a |
| 102 | XP_001389666.1 | 43.65 | Aspergillus niger CBS 513.88 |
| 102 | XP_006968965.1 | 43.48 | Trichoderma reesei QM6a |
| 102 | XP_006965362.1 | 43.24 | Trichoderma reesei QM6a |
| 102 | XP_003666790.1 | 43.08 | Thermothelomyces thermophilus |
| | | | ATCC 42464 |
| 102 | XP_001401064.2 | 43.04 | Aspergillus niger 513.88 |
| 102 | XP_006969607.1 | 42.62 | Trichoderma reesei QM6a |
| 102 | XP_500843.1 | 42.05 | Yarrowia lipolytica CLIB122 |
| 102 | XP_501891.1 | 41.67 | Yarrowia lipolytica CLIB122 |
| 102 | XP_003660835.1 | 41.51 | Thermothelomyces thermophilus |
| | | | ATCC 42464 |
| 102 | XP_500623.1 | 41.43 | Yarrowia lipolytica CLIB122 |
| 102 | XP_505948.1 | 41.24 | Yarrowia lipolytica CLIB122 |
| 102 | XP_003664877.1 | 41.18 | Thermothelomyces thermophilus |
| | | | ATCC 42464 |
| 102 | XP_001395019.1 | 41.18 | Aspergillus niger CBS 513.88 |
| 102 | XP_006968112.1 | 41.18 | Trichoderma reesei QM6a |
| 102 | XP_001824245.3 | 41.03 | Aspergillus oryzae RIB40 |
| 102 | XP_003664588.1 | 41.03 | Thermothelomyces thermophilus |
| | | | ATCC 42464 |
| 102 | XP_006961334.1 | 40.70 | Trichoderma reesie QM6a |
| 102 | XP_023091377.1 | 40.59 | Aspergillus oryzae RIB40 |
| 102 | XP_003664125.1 | 40.50 | Thermothelomyces thermophilus |
| | | | ATCC 42464 |
| 102 | XP_023094200.1 | 40.45 | Aspergillus oryzae RIB40 |
| 102 | XP_001401031.2 | 40.43 | Aspergillus niger CBS 513.88 |
| 102 | XP_504721.1 | 40.40 | Yarrowia lipolytica CLIB122 |
| 102 | XP_003661418.1 | 40.26 | Thermothelomyces thermophilus |
| | | | ATCC 42464 |
| 102 | XP_006965585.1 | 40.26 | Trichoderma reesei QM6a |
| 102 | XP_023090762.1 | 40.24 | Aspergillus oryzae RIB40 |
| 102 | XP_003659963.1 | 40.23 | Thermothelomyces thermophilus |
| | | | ATCC 42464 |
| 102 | XP_001827367.1 | 40.18 | Aspergillus oryzae RIB40 |
| 102 | XP_001817431.3 | 40.00 | Aspergillus oryzae RIB40 |
| 102 | XP_023089188.1 | 40.00 | Aspergillus oryzae RIB40 |
| 102 | XP_003659455.1 | 40.00 | Thermothelomyces thermophilus |
| | | | ATCC 42464 |
| 102 | XP_003658598.1 | 40.00 | Thermothelomyces thermophilus |
| | | | ATCC 42464 |
| 102 | XP_003662560.1 | 40.00 | Thermothelomyces thermophilus |
| | | | ATCC 42464 |
| 102 | XP_001396107.2 | 40.00 | Aspergillus niger CBS 513.88 |
| 106 | XP_006969575.1 | 100.00 | Trichoderma reesei QM6a |
| 106 | XP_023094297.1 | 23.00 | Aspergillus oryzae RIB40 |
| 106 | XP_001389373.2 | 42.24 | Aspergillus niger CBS 513.88 |
| 109 | XP_006969694.1 | 100.00 | Trichoderma reesei QM6a |
| 109 | XP_001727831.1 | 55.47 | Aspergillus oryzae RIB40 |
| 109 | XP_001401837.1 | 54.65 | Aspergillus niger CBS 513.88 |
| 109 | XP_001820478.1 | 53.78 | Aspergillus oryzae RIB40 |
| 109 | XP_001398591.1 | 50.00 | Aspergillus niger CBS 513.88 |
| 109 | XP_001826182.1 | 49.41 | Aspergillus oryzae RIB40 |
| 109 | XP_001398550.1 | 48.44 | Aspergillus niger CBS 513.88 |
| 109 | XP_505855.1 | 45.49 | Yarrowia lipolytica CLIB122 |
| 109 | XP_023091120.1 | 42.39 | Aspergillus oryzae RIB40 |
| 109 | XP_003660854.1 | 41.76 | Thermothelomyces thermophilus |
| | | | ATCC 42464 |
| 109 | XP_003660753.1 | 41.53 | Thermothelomyces thermophilus |
| | | | ATCC 42464 |
| 109 | XP_003662052.1 | 40.69 | Thermothelomyces thermophilus |
| | | | ATCC 42464 |
| 109 | XP_023093762.1 | 40.00 | Aspergillus oryzae RIB40 |
| 111 | XP_006961588.1 | 100.00 | Trichoderma reesei QM6a |
| 111 | XP_001393104.2 | 41.77 | Aspergillus niger CBS 513.88 |
| 114 | XP_006932953.1 | 100.00 | Trichoderma reesei QM6a |
| 114 | XP_006966914.1 | 48.03 | Trichoderma reesei QM6a |
| 114 | XP_001826195.3 | 43.26 | Aspergillus oryzae RIB40 |
| 114 | XP_003661372.1 | 42.65 | Thermothelomyces thermophilus |
| | | | ATCC 42464 |
| 114 | XP_023090190.1 | 42.57 | Aspergillus oryzae RIB40 |
| 114 | XP_003659247.1 | 42.33 | Thermothelomyces thermophilus |
| | | | ATCC 42464 |
| 114 | XP_006966793.1 | 40.87 | Trichoderma reesei QM6a |
| 117 | XP_006962953.1 | 100.00 | Trichoderma reesei QM6a |
| 117 | XP_006966914.1 | 48.033 | Trichoderma reesei QM6a |
| 117 | XP_023090190.1 | 42.57 | Aspergillus oryzae RIB40 |
| 117 | XP_003659247.1 | 42.12 | Thermothelomyces thermophilus |
| | | | ATCC 42464 |
| 117 | XP_006966793.1 | 40.89 | Trichoderma reesei QM6a |
| 120 | XP_006963776.1 | 100.00 | Trichoderma reesei QM6a |
| 120 | XP_023088815.1 | 47.87 | Aspergillus oryzae RIB40 |
| 120 | XP_001396231.2 | 47.70 | Aspergillus niger CBS 513.88 |
| 120 | XP_003664619.1 | 46.21 | Thermothelomyces thermophilus |
| | | | ATCC 42464 |
| 120 | XP_006967198.1 | 45.91 | Trichoderma reesei QM6a |
| 123 | XP_006964065.1 | 100.00 | Trichoderma reesei QM6a |
| 126 | XP_006965391.1 | 100.00 | Trichoderma reesei QM6a |
| 129 | XP_006966768.1 | 100.00 | Trichoderma reesei QM6a |
| 129 | XP_003658630.1 | 57.41 | Thermothelomyces thermophilus |
| | | | ATCC 42464 |
| 129 | XP_001819677.1 | 55.24 | Aspergillus oryzae RIB40 |
| 129 | XP_001389683.1 | 54.19 | Aspergillus niger CBS 513.88 |
| 129 | XP_505289.1 | 53.82 | Yarrowia lipolytica CLIB122 |
| 129 | ANZ75707.1 | 49.53 | Komagataella pastoris |
| 129 | NP_013507.1 | 40.16 | Saccharomyces cerevisiae S288C |
| 13 | XP_006968743.1 | 100.00 | Trichoderma reesei QM6a |

### Example 11

### Improved Sporulation by Overexpression of a Regulatory Protein

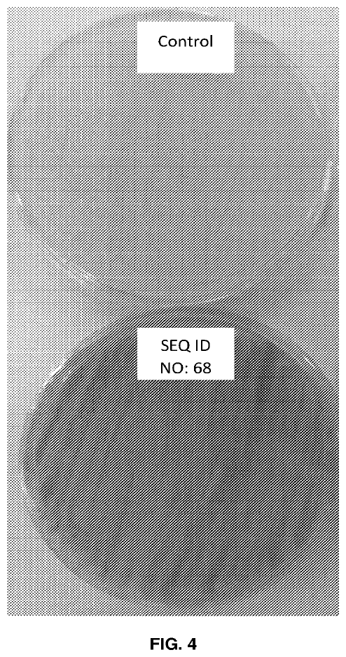

In the present example, the recombinant strain overexpressing the regulatory protein SEQ ID NO: 68 and the control strain (i.e., not overexpressing any regulatory proteins) were plated on agar plate minimal medium (20 g/L agar, 7 g/L (NH₄)₂SO₄, 4.7 g/L KH₂PO4, 1 g/L MgSO₄·7H₂O, 0.6 g/L CaCl₂·2H₂O, at pH 5.5, 0.25% T. reesei trace elements; 175 g/L C₆H₈O₇, 200 g/L FeSO₄·7H₂O, 16 g/L ZnSO₄·7H₂O, 3.2 g/L CuSO₄·5H₂O, 1.4 g/L MnSO₄·H₂O and 0.8 g/L H₃BO₃) with 2.0% (w/w) glucose as a carbon source. Plates were incubated at 28° C. in light incubator (12 h/12 h light cycle) for 72 hours. For example, as shown in FIG. 4, the recombinant strain overexpressing the regulatory protein of SEQ ID NO: 68 showed a higher sporulation level than the control strain.

### Example 12

### Elevated Background Protease Activity by Overexpression of Regulatory Proteins

In the instant example, all six-hundred ninety-one (691) regulatory protein overexpression mutants described above (Example 4) were monitored for increased endogenous proteolytic activity. More particularly, fermentation samples generated from lactose containing production medium at 28° C. were spotted on indicator agar plates with 1% casein. Proteolytic activity from samples that formed visual halo caused by precipitation of degraded casein was measured quantitatively using Pierce Protease Assay kit 23263 (Thermo Scientific) according to recommendations of the supplier. Proteolytic activity was measured at two (2) different pH and was referred to that from the control strain.

As presented below in TABLE 7, recombinant strains overexpressing the regulatory protein SEQ ID NO: 4, SEQ ID NO: 99, SEQ ID NO: 134, SEQ ID NO: 137, SEQ ID NO: 140, SEQ ID NO:144, SEQ ID NO: 147, SEQ ID NO: 149 and SEQ ID NO: 152, showed increased background proteolytic activity, indicating that the abovementioned regulatory factors are involved in regulation of protease gene expression. Applicant contemplates herein that genetic modification (e.g., deletion, disruption, etc.) of one or more regulatory genes set forth in TABLE 7 are useful in reducing/mitigating proteolysis of proteins of interest, which are particularly susceptible to proteolytic degradation.

TABLE 7
Relative Protease Activity in Fermented Cultures of Strains Overexpressing
Regulatory Proteins Indicated by SEQ Identification Number

| | | SEQ | SEQ | SEQ | SEQ | SEQ | SEQ | SEQ | SEQ | SEQ |
|---|---|---|---|---|---|---|---|---|---|---|
| Strain | Control | 134 | 147 | 152 | 140 | 137 | 4 | 99 | 149 | 144 |
| PI at | 1.00 | 3.57 | 2.85 | 2.46 | 1.76 | 2.56 | 5.88 | 3.15 | 2.43 | 1.85 |
| pH 4.0 | | | | | | | | | | |
| PI at | 1.00 | 3.89 | 1.17 | 1.67 | 0.53 | 1.16 | 3.90 | 5.71 | 1.42 | 3.02 |
| pH 7.5 | | | | | | | | | | |

### REFERENCES

EPO Publication No. 0215594
PCT Publication No. WO 2021/102238
PCT Publication No. WO2008/097619
PCT Publication No. WO2005/056772
PCT Publication No. WO2008/080017
PCT Publication No. WO2011/151512
PCT Publication No. WO2011/151513
PCT Publication No. WO2011/151515
PCT Publication No. WO2011/153449
PCT Publication No. WO2013/102674
PCT Publication No. WO2014/093275
PCT Publication No. WO2016/100272
U.S. Pat. No. 6,022,725
U.S. Pat. No. 6,255,115
U.S. Pat. No. 6,268,328
Aro et al., “Transcriptional regulation of plant cell wall degradation by filamentous fungi” FEMS Microbiology Reviews 29: 719-739. 2005.
Ausubel et al., “Current Protocols in Molecular Biology, published by Greene Publishing Assoc. and Wiley-Interscience (1987, 1989 and 2003).
Boel et al., “Two different types of intervening sequences in the glucoamylase gene from Aspergillus niger”, EMBO J. 3:1581-1585, 1984.
Campbell et al., Curr. Genet., 16: 53-56, 1989.
Cao et al., “Penicillopepsin-JT2, a recombinant enzyme from Penicillium janthinellum and the contribution of a hydrogen bond in subsite S3 to kcat”, Protein Science (9):991-1001, 2000.
Devereux et al., Nucleic Acids Res. 12:387-395, 1984.
Druzhinina and Kubicek, “Genetic engineering of T. reesei cellulases and their production”, Microb. Biotechnol. 10, 1485-1499, 2017.
Fitz et al. “The Promoter Toolbox for Recombinant Gene Expression in Trichoderma reesei”, Frontiers in Bioengineering and Biotechnology, Volume 6, Article 135, 2018.
Harkki et al., “A Novel Fungal Expression System: Secretion of Active Calf Chymosin from the Filamentous Fungus Trichoderma reesei”, BioTechnol., 7: 596-603, 1989.
Harkki et al., “Genetic engineering of Trichoderma to produce strains with novel cellulase profiles”, Enzyme Microb. Technol., 13: 227-233, 1991.
Ilmen et al., “Regulation of cellulase gene expression in the filamentous fungus Trichoderma reesei”, Applied and Environmental Microbiology, 63(4)-1298-1306, 1997.
Kriegler, Gene Transfer and Expression: A Laboratory Manual, 1990.
Kubicek et al., “Metabolic engineering strategies for the improvement of cellulase production by Hypocrea jecorina”, Biotechnol. Biofuels 2:19, 2009.
Latchman, “Transcription factors: an overview”, Int. J. Exp. Path., 74, 417-422, 1993.
Lever, “A new reaction for colorimetric determination of carbohydrates”, Anal. Biochem., 47:273-279, 1972.
Li, Wan-Chen et al. “Trichoderma reesei complete genome sequence, repeat-induced point mutation, and partitioning of CAZyme gene clusters.” Biotechnology for biofuels Vol. 10 (170), 2017.
Mullaney et al., “Primary structure of the trpC gene from Aspergillus nidulans”, Molecular and General Genetics 199:37-45, 1985.
Needleman and Wunsch, “A general method applicable to the search for similarities in the amino acid sequence of two proteins”, J. Mol. Biol. 48: 443-453, 1970.
Nordberg et al., “The genome portal of the Department of Energy Joint Genome Institute: 2014 updates”, Nucleic Acids Res., 42(1):D26-31, 2014.
Nunberg et al., “Molecular cloning and characterization of the glucoamylase gene of Aspergillus awamori”, Mol. Cell Biol. 4:2306, 1984.
Pearson and Lipman, Proc. Natl. Acad. Sci. USA 85:2444, 1988.
Portnoy et al., “Differential Regulation of the Cellulase Transcription Factors XYR1, ACE2, and ACE1 in Trichoderma reesei Strains Producing High and Low Levels of Cellulase”, Eukaryotic Cell, 10(2): 262-271, 2011.
Rice et al., “EMBOSS: The European Molecular Biology Open Software Suite”, Trends in Genetics 16: 276-277, 2000.
Sambrook et al., Molecular Cloning, A Laboratory Manual, 2^{nd} Edition, Cold Spring Harbor Laboratory Press, Cold Spring, New York, 1989.
Sambrook et al., Molecular Cloning, A Laboratory Manual, 4^{th} Edition. Cold Spring Harbor Laboratory Press, Cold Spring, New York, 2012.
Sheir-Neiss and Montenecourt, “Characterization of the secreted cellulases of Trichoderma reesei wild type and mutants during controlled fermentations”, Applied Microbiology and Biotechnology, 20(1):46-53, 1984.
Smith and Waterman, Adv. Appl. Math. 2:482, 1981.
Stricker et al., “Regulation of transcription of cellulases- and hemicellulases-encoding genes in Aspergillus niger and Hypocrea jecorina (Trichoderma reesei), Appl. Microbiol. Biotechnol. (Review), 78(2):211-220, 2008.
Wang et al., “Fermentation & Enzyme Technology”, John Wiley & Sons, 1979.
Wood, “The cellulase of Fusarium solani. Purification and specificity of the 3-(1→4)-glucanase and the β-d-glucosidase components”, Biochem. J., 121:353-362, 1971.
Wu et al., “The regulatory and transcriptional landscape associated with carbon utilization in a filamentous fungus”, PNAS, 117(11): 6003-6013, 2020.
Yelton et al., “Transformation of Aspergillus nidulans by using a trpC plasmid”, PNAS USA 81:1470-1474, 1984.

1. A recombinant fungal cell overexpressing a gene comprising at least 80% identity to a Trichoderma reesei gene of SEQ ID NO: 1, 4, 7, 10, 12, 15, 18, 21, 23, 25, 28, 30, 33, 36, 39, 42, 45, 48, 51, 54, 56, 59, 62, 65, 68, 71, 74, 77, 79, 82, 84, 87, 90, 94, 97, 99, 101, 105, 108, 110, 113, 116, 119, 122, 125, 128 or 131.

2. (canceled)

3. The recombinant fungal cell of claim 1, wherein the overexpressed gene encodes a protein comprising at least 80% identity to a Trichoderma reesei protein of SEQ ID NO: 2, 5, 8, 11, 13, 16, 19, 22, 24, 26, 29, 31, 34, 37, 40, 43, 46, 49, 52, 55, 57, 60, 63, 66, 69, 72, 75, 78, 80, 83, 85, 88, 91, 95, 98, 100, 102, 106, 109, 111, 114, 117, 120, 123, 126, 129 or 132, respectively.

4. The recombinant fungal cell of claim 1, wherein the cell comprises an introduced polynucleotide cassette encoding a protein of interest (POI), wherein the cassette comprises an upstream cellulase gene promoter operably linked to a downstream nucleic acid encoding the POI.

5. The recombinant fungal cell of claim 4 overexpressing a combination of at least two genes comprising at least 80% identity to a Trichoderma reesei gene combination of SEQ ID NO: 4 and 30, SEQ ID NO: 4 and 94, SEQ ID NO: 4 and 97, SEQ ID NO: 4 and 99, SEQ ID NO: 12 and 12, SEQ ID NO: 12 and 30, SEQ ID NO: 12 and 68, SEQ ID NO: 23 and 101, SEQ ID NO: 23 and 113, SEQ ID NO: 23 and 12, SEQ ID NO: 23 and 4, SEQ ID NO: 23 and 94, SEQ ID NO: 23 and 97, SEQ ID NO: 23 and 99, SEQ ID NO: 25 and 101, SEQ ID NO: 25 and 12, SEQ ID NO: 25 and 30, SEQ ID NO: 25 and 65, SEQ ID NO: 25 and 68, SEQ ID NO: 25 and 94, SEQ ID NO: 30 and 12, SEQ ID NO: 30 and 23, SEQ ID NO: 30 and 30, SEQ ID NO: 30 and 65, SEQ ID NO: 30 and 68, SEQ ID NO: 30 and 94, SEQ ID NO: 30 and 97, SEQ ID NO: 30 and 99, SEQ ID NO: 65 and 12, SEQ ID NO: 65 and 30, SEQ ID NO: 65 and 4, SEQ ID NO: 65 and 65, SEQ ID NO: 65 and 68, SEQ ID NO: 65 and 94, SEQ ID NO: 65 and 97, SEQ ID NO: 65 and 99, SEQ ID NO: 87 and 30, SEQ ID NO: 94 and 30, SEQ ID NO: 94 and 68, SEQ ID NO: 94 and 94, SEQ ID NO: 97 and 12, SEQ ID NO: 97 and 23, SEQ ID NO: 97 and 30, SEQ ID NO: 97 and 4, SEQ ID NO: 97 and 68, SEQ ID NO: 97 and 94, SEQ ID NO: 97 and 97, SEQ ID NO: 97 and 99, SEQ ID NO: 99 and 30, SEQ ID NO: 99 and 65, SEQ ID NO: 99 and 68, SEQ ID NO: 99 and 97, SEQ ID NO: 110 and 30, SEQ ID NO: 110 and 65, SEQ ID NO: 110 and 68, SEQ ID NO: 128 and 101, SEQ ID NO: 128 and 12, SEQ ID NO: 128 and 94, or SEQ ID NO: 128 and 97.

6. The recombinant fungal cell of claim 4 overexpressing a combination of at least two genes comprising at least 80% identity to a Trichoderma reesei gene combination of SEQ ID NO: 12 and 101, SEQ ID NO: 12 and 128, SEQ ID NO: 12 and 23, SEQ ID NO: 12 and 65, SEQ ID NO: 12 and 99, SEQ ID NO: 25 and 101, SEQ ID NO: 30 and 101, SEQ ID NO: 30 and 12, SEQ ID NO: 30 and 23, SEQ ID NO: 30 and 65, SEQ ID NO: 30 and 94, SEQ ID NO: 30 and 97, SEQ ID NO: 87 and 113, SEQ ID NO: 87 and 99, SEQ ID NO: 94 and 101, SEQ ID NO: 94 and 12, SEQ ID NO: 94 and 23, SEQ ID NO: 94 and 30, SEQ ID NO: 94 and 4, SEQ ID NO: 94 and 68, SEQ ID NO: 94 and 94, SEQ ID NO: 94 and 99, SEQ ID NO: 97 and 101, SEQ ID NO: 97 and 113, SEQ ID NO: 97 and 30, SEQ ID NO: 97 and 99, SEQ ID NO: 99 and 101, SEQ ID NO: 99 and 12, SEQ ID NO: 99 and 23, SEQ ID NO: 99 and 30, SEQ ID NO: 99 and 65, SEQ ID NO: 99 and 68, SEQ ID NO: 99 and 94, SEQ ID NO: 99 and 97, SEQ ID NO: 99 and 99, or SEQ ID NO: 110 and 101.

7-9. (canceled)

10. A recombinant fungal cell overexpressing a protein comprising at least 80% identity to SEQ ID NO: 68, wherein the recombinant cell comprises an enhanced sporulation phenotype.

11-23. (canceled)

24. A method for producing an increased amount of a heterologous protein of interest (POI) in a fungal cell comprising:
(a) constructing a recombinant fungal cell comprising an introduced expression cassette encoding the POI, wherein the cassette comprises an upstream cellulase gene promoter operably linked to a downstream nucleic acid encoding the POI,
(b) introducing into the cell a polynucleotide comprising an upstream heterologous promoter operably linked to a downstream nucleic acid encoding a protein comprising at least 80% sequence identity to a protein of SEQ ID NO: 2, 5, 8, 11, 13, 16, 19, 22, 24, 26, 29, 31, 34, 37, 40, 43, 46, 49, 52, 55, 57, 60, 63, 66, 69, 72, 75, 78, 80, 83, 85, 88, 91, 95, 98, 100, 102, 106, 109, 111, 114, 117, 120, 123, 126, 129 or 132, and
(c) fermenting the recombinant cell,
wherein the recombinant cell of step (b) produces an increased amount of the POI relative to the recombinant cell of step (a), when the cells are fermented under the same conditions for the production of the PO1.

25-27. (canceled)

28. The method of claim 24, wherein the polynucleotide encoding the POI is integrated into the genome of the fungal cell and/or the polynucleotide encoding the regulatory protein is integrated into the genome of the fungal cell.

29. The method of claim 24, wherein the POI is an enzyme.

30. (canceled)

31. The method of claim 29, wherein the enzyme is an amylase or a phytase.

32. A method for enhancing sporulation of a fungal cell comprising:
(a) constructing or obtaining a recombinant fungal cell overexpressing a protein comprising at least 80% identity to SEQ ID NO: 68, and
(b) growing strain under appropriate sporulation conditions,
wherein the recombinant cell comprises an enhanced sporulation phenotype as compared to a control fungal cell which does not overexpress the protein comprising at least 80% identity to SEQ ID NO: 68.
